(19) United States
(12) Patent Application Publication
(10) Pub. No.: US 2025/0304726 A1
(43) Pub. Date: Oct. 2, 2025

(54) NITRIC OXIDE-RELEASING GLYCOSAMINOGLYCANS FOR WOUND HEALING

(71) Applicant: The University of North Carolina at Chapel Hill, Chapel Hill, NC (US)

(72) Inventors: Mark H. SCHOENFISCH, Chapel Hill, NC (US); Sara MALONEY, Chapel Hill, NC (US)

(73) Assignee: The University of North Carolina at Chapel Hill, Chapel Hill, NC (US)

(21) Appl. No.: 18/864,506

(22) PCT Filed: May 10, 2023
(86) PCT No.: PCT/US2023/021749

§ 371 Date: Nov. 8, 2024

Related U.S. Application Data
(60) Provisional application No. 63/364,544, filed on May 11, 2022.

(51) Int. Cl. C08B 37/00; A61K 31/726; A61P 31/02

(52) U.S. Cl. CPC C08B 37/0063; A61K 31/726; A61P 31/02

(57) ABSTRACT

Described herein are NO-releasing chondroitin sulfate (CS) polymers, methods for preparing the polymers, and their use for the treatment of various medical conditions, such as infected wounds.

### CROSS-REFERENCE TO RELATED APPLICATIONS

This application claims the benefit of and priority to U.S. Provisional Patent Application No. 63/364,544, filed on May 11, 2022, the entire contents of which are incorporated herein by reference.

### GOVERNMENT INTEREST

This invention was made with government support under Grant Nos. DK108318 and DE025207 awarded by the National Institutes of Health. The government has certain rights in the invention.

### FIELD

The presently disclosed subject matter relates generally to nitric oxide-releasing polymers and scaffolds made therefrom that store and/or release nitric oxide in a controlled manner. Additionally disclosed are methods of synthesis of the same and methods of use of the same as antibacterial agents in methods of treatment, particularly in the treatment of wounds.

### BACKGROUND

Approximately 2% of the total population in the United States is living with a chronic wound, loosely defined as a skin trauma that fails to heal in a normal timeframe.¹⁻⁴ These wounds often stall in a state of inflammation as a result of persistent stimuli, including prolonged pressure or repetitive tissue trauma, or due to underlying conditions, such as those that impair the host immune response (e.g., diabetes, cancer).⁵⁻⁷ The delayed healing observed for chronic wounds is often potentiated by wound infection, especially in the case of biofilm formation. Biofilms, or cooperative communities of bacteria that self-secrete an exopolysaccharide (EPS) matrix, are difficult to eradicate with conventional antibiotics due to changes in bacterial metabolism and the presence of a viscous matrix that prevents antibiotic diffusion.⁸⁻¹³ Further, bacteria are able to develop resistance to conventional antibiotics, rendering many current antibacterial therapies ineffective.^{14,15} Due to the devastating ramifications of nonhealing wounds, including pain, amputation, and even death, new wound treatments that address both infection and impairment of the host immune response, without the potential resistance, are greatly needed. The subject matter described herein addresses this unmet need.

### BRIEF SUMMARY

In one aspect, the presently disclosed subject matter is directed to a NO-releasing polymer compound comprising a unit structure of Formula I.

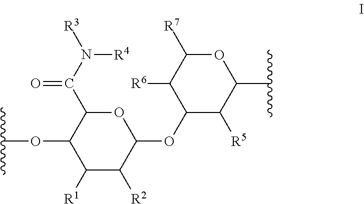

wherein,
R¹, R², and R⁵ are each independently selected from the group consisting of —OH, —NH₂, —CH₂OH, —C(O)OH, —NHC(O)—CH₃, C₁-C₆ alkoxy, —O—((CH₂)ₐO)_{b}—(CH₂)_{c}H, —NH—((CH₂)_{d}NH)ₑ—(CH₂)_{f}H, —X¹—((CH₂)_{g}X²)ₕ—(CH₂)ᵢH, —CH₂C(O)—X¹—((CH₂)_{g}X²)ₕ((CH₂)ⱼX³)ₖ(CH₂)H, —X¹—((CH₂)_{g}X²)ₕ((CH₂)ⱼX³)ₖ—(CH₂)H, —X¹—(CH₂)_{g}X⁴, —CH₂C(O)—X¹—((CH₂)_{g}X²)ₕ(CH₂)ⱼX⁵, and —X¹—((CH₂)_{g}X²)ₕ(CH₂)ⱼX⁶;
R⁶ and R⁷ are each independently selected from the group consisting of —OH, —NH₂, —CH₂OH, —C(O)OH, C₁-C₆ alkoxy, —CH₂OSO₃, and —OSO₃, provided that at least one of R₆ and R₇ is —CH₂OSO₃⁻ or —OSO₃;
R³ is selected from the group consisting of —((CH₂)_{g}X²)ₕ—(CH₂)ᵢH, —((CH₂)_{g}X²)ₕ((CH₂)ⱼX³)ₖ—(CH₂)H, —(CH₂)_{g}X⁴, and —((CH₂)_{g}X²)ₕ(CH₂)ⱼX⁵;
R⁴ is selected from the group consisting of hydrogen and C₁-C₆ alkyl;
   a, b, c, d, e, f, g, h, i, j, k, and l are each independently an integer between 0 and 10;
   X¹, X², and X³ are each independently selected from the group consisting of —O—, —S—, —NH, —C(O)NH—, and a first NO donating moiety selected from the group consisting of

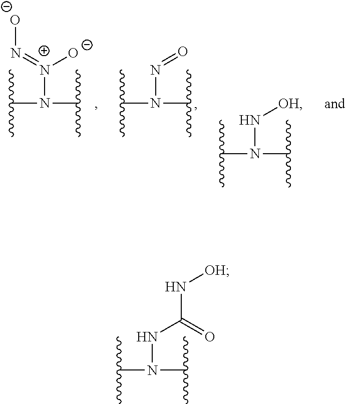 [SMILES: *N(*)/[N+]([O-])=N/[O-] ; *N(*)N=O ; *N(*)NO ; *N(*)NC(=O)NO]

X⁴, X⁵, and X⁶ are each independently selected from a second NO donating moiety selected from the group consisting of and

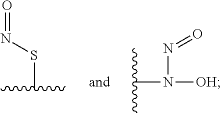

provided that the NO-releasing polymer compound contains at least one first NO donating moiety or second NO donating moiety.

In another aspect, the subject matter described herein is directed to a method of treating a wound infected with a bacterial pathogen, comprising contacting the wound with an effective amount of an NO-releasing polymer compound described herein.

These and other aspects are described herein.

### BRIEF DESCRIPTION OF THE DRAWINGS

FIG. 1 shows representative ¹H NMR (600 MHz, D₂O) of unmodified and amine-modified (A) HA6, (B) HA50, (C) HA90, (D) CSA, and (E) CSC derivatives.

FIG. 2 shows representative ¹³C NMR (600 MHz, D₂O) of unmodified and amine-modified (A) HA6, (B) HA50, (C) HA90, (D) CSA, and (E) CSC derivatives.

FIG. 3 shows representative FTIR analysis of unmodified and amine-modified (A) HA6, (B) HA50, (C) HA90, (D) CSA, and (E) CSC derivatives.

FIG. 4 shows representative UV-Vis spectra of control (− −) and NO-releasing (−) GAG derivatives modified with HEDA, DPTA, or DETA. Spectra show (A) HA6, (B) HA50, (C) HA90, (D) CSA, and (E) CSC.

FIG. 5 shows plots of (A) real-time release profile over initial 30 min and (B) cumulative NO release over release duration of HA6-HEDA/NO, HA6-DPTA/NO, and HA6-DETA/NO in PBS (solid) and SWF (dashed); (C) real-time release profile over initial 30 min and (D) cumulative NO release over release duration of CSC-HEDA/NO, CSC-DPTA/NO, and CSC-DETA/NO in PBS (solid) and SWF (dashed).

FIG. 6 shows plots of antibacterial efficacy of NO-releasing GAG derivatives against (A, C, E) PAO1 and (B, D, F) ATCC S. aureus. Modifications of HA6 (circle), HA50 (triangle), HA90 (square), CSA (hexagon), and CSC (diamond) include (A-B) HEDA, (C-D) DPTA, and (E-F) DETA. Error bars represent the standard deviation for n≥3 separate experiments.

FIG. 7 shows plots of antibacterial efficacy of NO-releasing GAG derivatives against (A, C, E) ATCC MDR-PA and (B, D, F) ATCC MRSA. Modifications of HA6 (circle), HA50 (triangle), HA90 (square), CSA (hexagon), and CSC (diamond) include (A-HEDA, (C-D) DPTA, and (E-F) DETA. Error bars represent the standard deviation for n≥3 separate experiments.

FIG. 8 shows plots of antibacterial efficacy of NO-releasing GAG derivatives against CDC multidrug resistant isolates (A, C, E) AR-0239 and (B, D, F) AR-0565. Modifications of HA6 (circle), HA50 (triangle), HA90 (square), CSA (hexagon), and CSC (diamond) include (A-B) HEDA, (C-D) DPTA, and (E-F) DETA. Error bars represent the standard deviation for n≥3 separate experiments.

FIG. 9 shows plots of colonies of (A) PAO1, (B) ATCC S. aureus, (C) ATCC MDR-PA, (D) ATCC MRSA, (E) AR-0239, and (F) AR-0565 remaining after 4-h treatment with amine-modified GAGs (without NO). All modifications were evaluated at 16 mg mL⁻¹. Error bars represent the standard deviation for n≥3 separate experiments.

FIG. 10 shows dose-response curves for unmodified glycosaminoglycans against human dermal fibroblasts (A-B) and human epidermal keratinocytes (C-D). Glycosaminoglycan derivatives include HA6 (circle), HA50 (triangle), HA90 (square), CSA (hexagon), and CSC (diamond). Error bars represent the standard deviation for n≥3 separate experiments.

FIG. 11 shows dose-response curves after 24-h treatment of human dermal fibroblasts with amine-modified (hollow) and NO-releasing (solid) glycosaminoglycan derivatives. Modifications of HA6 (circle), HA50 (triangle), HA90 (square), CSA (hexagon), and CSC (diamond) include (A-B) HEDA, (C-D) DPTA, and (E-F) DETA. Error bars represent the standard deviation for n≥3 separate experiments.

FIG. 12 shows dose-response curves after 24-h treatment of human epidermal keratinocytes with amine-modified (hollow) and NO-releasing (solid) glycosaminoglycan derivatives. Modifications of HA6 (circle), HA50 (triangle), HA90 (square), CSA (hexagon), and CSC (diamond) include (A-B) HEDA, (C-D) DPTA, and (E-F) DETA. Error bars represent the standard deviation for n≥3 separate experiments.

FIG. 13 shows a plot of the concentration of amine-modified (solid) or NO-releasing (striped) glycosaminoglycan derivatives required to inhibit metabolic activity of (A) human dermal fibroblasts (HDFs) or (B) human epidermal keratinocytes (HEKs) by 50% (IC₅₀).

FIG. 14 shows a bar graph of activation of NF-κB in HEK-BLUE mTLR4 cells upon treatment with LPS or TNF-α at concentrations of 0.1 to 100 ng mL⁻¹. NF-κB-induced SEAP activity is reported as the OD630 corrected for blank media. Error bars represent the standard deviation of n≥3 separate experiments.

FIG. 15 shows a bar graph of activation of NF-κB via murine TLR4 receptor in HEK-BLUE mTLR4 cells upon treatment with unmodified GAGs at concentrations of (A) 100 ng mL⁻¹, (B) 1 μg mL⁻¹, (C) 10 μg mL⁻¹, and (D) 100 μg mL⁻¹. NF-κB-induced SEAP activity is reported as the OD630 corrected for blank media. Error bars represent the standard deviation of n≥3 separate experiments. Of note, the y-axis of (D) uses a different range to present the data. * p<0.05 compared to untreated cells.

FIG. 16 shows a bar graph of activation of NF-κB via murine TLR4 receptor in HEK-BLUE mTLR4 cells upon treatment with amine-modified (solid) or NO-releasing (striped) GAG derivatives at a concentration of 100 μg mL⁻¹. NF-κB-induced SEAP activity is reported as the OD₆₃₀ corrected for blank media. Error bars represent the standard deviation of n≥3 separate experiments. * p<0.05 compared to untreated cells.

FIG. 17 shows bar graphs of activation of NF-κB via murine TLR4 receptor in HEK-BLUE mTLR4 cells upon treatment with amine-modified (solid) or NO-releasing (striped) GAG derivatives at concentrations of (A) 100 ng mL⁻¹, (B) 1 μg mL⁻¹, or (C) 10 μg mL⁻¹. NF-κB-induced SEAP activity is reported as the OD630 corrected for blank media. Error bars represent the standard deviation of n≥3 separate experiments. * p<0.05 compared to untreated cells.

FIG. 18 shows a bar graph of activation of NF-κB in HEK-BLUE Null1-v cells upon treatment with LPS or TNF-α at concentrations of 0.1 to 100 ng mL⁻¹. Cells express endogenous levels of TLR3, TLR5, NOD1, ALPK1, and TIFA but are not transfected with the murine TLR4 receptor gene. NF-κB-induced SEAP activity is reported as the OD630 corrected for blank media. Error bars represent the standard deviation of n=3 separate experiments.

FIG. 19 shows bar graphs of activation of NF-κB in HEK-BLUE Null1-v cells upon treatment with unmodified GAGs at concentrations of (A) 100 ng mL⁻¹, (B) 1 μg mL⁻¹, (C) 10 μg mL⁻¹, and (D) 100 μg mL⁻¹. Cells express endogenous levels of TLR3, TLR5, NOD1, ALPK1, and TIFA but are not transfected with the murine TLR4 receptor gene. NF-κB-induced SEAP activity is reported as the OD630 corrected for blank media. Error bars represent the standard deviation of n=3 separate experiments.

FIG. 20 shows bar graphs of activation of NF-κB in HEK-BLUE Null1-v cells upon treatment with amine-modified (solid) or NO-releasing (striped) GAG derivatives at concentrations of (A) 100 ng mL⁻¹, (B) 1 μg mL⁻¹, (C) 10 μg mL⁻¹, and (D) 100 μg mL⁻¹. Cells express endogenous levels of TLR3, TLR5, NOD1, ALPK1, and TIFA but are not transfected with the murine TLR4 receptor gene. NF-κB-induced SEAP activity is reported as the OD630 corrected for blank media. Error bars represent the standard deviation of n=3 separate experiments.

FIG. 21 shows bar graphs of adhesion of HDFs treated with unmodified GAGs at concentrations of (A) 100 ng mL⁻¹, (B) 1 μg mL⁻¹, (C) 10 μg mL⁻¹, and (D) 100 μg mL⁻¹. Adhesion of GAG derivatives is reported as a percentage of the total number of cells seeded in each well. Error bars represent the standard deviation of n≥4 separate experiments. * p<0.05 compared to untreated cells.

FIG. 22 shows bar graphs of adhesion of HEKs treated with unmodified GAGs at concentrations of (A) 100 ng mL⁻¹, (B) 1 μg mL⁻¹, (C) 10 μg mL⁻¹, and (D) 100 μg mL⁻¹. Adhesion of GAG derivatives is reported as a percentage of the total number of cells seeded in each well. Error bars represent the standard deviation of n≥4 separate experiments. * p<0.05 compared to untreated cells.

FIG. 23 shows bar graphs of adhesion of HDFs treated with amine-modified (solid) or NO-releasing (striped) GAG derivatives at concentrations of (A) 100 ng mL⁻¹, (B) 1 μg mL⁻¹, and (C) 100 μg mL⁻¹. Adhesion of GAG derivatives is reported as a percentage of the total number of cells seeded in each well. Error bars represent the standard deviation of n≥5 separate experiments. * p<0.05 compared to untreated cells.

FIG. 24 shows bar graphs of (A, B) adhesion and (C, D) relative proliferation of (A, C) HDFs and (B, D) HEKs treated with amine-modified (solid) or NO-releasing (striped) GAG derivatives at a concentration of 10 μg mL⁻¹. Adhesion of GAG derivatives is reported as a percentage of the total number of cells seeded in each well. Proliferation of GAG derivatives is reported relative to untreated cells (set to 100% proliferation). Error bars represent the standard deviation of n≥4 separate experiments. * p<0.05 compared to untreated cells. Note: the y-axis is scaled appropriately for each data set.

FIG. 25 shows bar graphs of adhesion of HEKs treated with amine-modified (solid) or NO-releasing (striped) GAG derivatives at concentrations of (A) 100 ng mL⁻¹, (B) 1 μg mL⁻¹, and (C) 100 μg mL⁻¹. Adhesion of GAG derivatives is reported as a percentage of the total number of cells seeded in each well. Error bars represent the standard deviation of n≥5 separate experiments. * p<0.05 compared to untreated cells.

FIG. 26 shows bar graphs of proliferation of HDFs treated with unmodified GAGs at concentrations of (A) 100 ng mL⁻¹, (B) 1 μg mL⁻¹, (C) 10 μg mL⁻¹, and (D) 100 μg mL⁻¹. Proliferation of GAGs is reported relative to untreated cells (set to 100% proliferation). Error bars represent the standard deviation of n≥4 separate experiments. * p<0.05 compared to untreated cells.

FIG. 27 shows bar graphs of proliferation of HEKs treated with unmodified GAGs at concentrations of (A) 100 ng mL⁻¹, (B) 1 μg mL⁻¹, (C) 10 μg mL⁻¹, and (D) 100 μg mL⁻¹. Proliferation of GAGs is reported relative to untreated cells (set to 100% proliferation). Error bars represent the standard deviation of n≥3 separate experiments. * p<0.05 compared to untreated cells.

FIG. 28 shows bar graphs of proliferation of HDFs treated with amine-modified (solid) or NO-releasing (striped) GAG derivatives at concentrations of (A) 100 ng mL⁻¹, (B) 1 μg mL⁻¹, and (C) 100 μg mL⁻¹. Proliferation of GAG derivatives is reported relative to untreated cells (set to 100% proliferation). Error bars represent the standard deviation of n≥4 separate experiments. * p<0.05 compared to untreated cells.

FIG. 29 shows bar graphs of proliferation of HEKs treated with amine-modified (solid) or NO-releasing (striped) GAG derivatives at concentrations of (A) 100 ng mL⁻¹, (B) 1 μg mL⁻¹, and (C) 100 μg mL⁻¹. Proliferation of GAG derivatives is reported relative to untreated cells (set to 100% proliferation). Error bars represent the standard deviation of n≥4 separate experiments. * p<0.05 compared to untreated cells.

FIG. 30 shows heat maps displaying a positive (110-129, 130-149, or >150), negative (50-69 or 70-89), or neutral (90-109) effect on HDF and HEK adhesion and proliferation following treatment with (A) 100 ng mL⁻¹, (B) 1 μg mL⁻¹, (C) 10 μg mL⁻¹, or (D) 100 μg mL⁻¹ of amine-modified (control) or NO-releasing GAGs. Data represents the average of n≥3 separate experiments.

FIG. 31 shows bar graphs of (A) percentage of initial wound area remaining following daily treatment with PEG (solid), 50 mg kg⁻¹ of HA6-HEDA/NO in PEG (starred), or 50 mg kg⁻¹ of CSC-HEDA/NO in PEG (dashed). Of note, mice were treated and imaged on day 5 post-wounding but not measured. (B) Percentage of initial wound area remaining following daily treatment with PEG (first bar graph in each set from left to right), 50 mg kg⁻¹ of CSC-HEDA in PEG (second bar graph in each set from left to right), 50 mg kg⁻¹ of CSC-HEDA/NO in PEG (third bar graph in each set from left to right), 50 mg kg⁻¹ of CSC-DPTA in PEG (fourth bar graph in each set from left to right), or 50 mg kg⁻¹ of CSC-DPTA/NO in PEG (fifth bar graph in each set from left to right). (C) Representative images of wounds from each treatment group. Error bars represent the standard deviation for n=5 mice. *p<0.05, **p<0.01, ***p<0.005.

FIG. 32 shows bar graphs of relative quantity of P. aeruginosa genome remaining in wound tissue harvested 8 days post-wounding for mice treated daily with (A) PEG (first bar graph from left to right), 50 mg kg⁻¹ of HA6-HEDA/NO in PEG (second bar graph from left to right), or 50 mg kg⁻¹ of CSC-HEDA/NO in PEG (third bar graph from left to right), or (B) PEG (first bar graph from left to right), 50 mg kg⁻¹ of CSC-HEDA in PEG (second bar graph from left to right), 50 mg kg⁻¹ of CSC-HEDA/NO in PEG (third bar graph from left to right), 50 mg kg⁻¹ of CSC-DPTA in PEG (fourth bar graph from left to right), or 50 mg kg⁻¹ of CSC-DPTA/NO in PEG (fifth bar graph from left to right). Error bars represent the standard deviation for n=5 mice. *p<0.05, ** p<0.01, *** p<0.005.

### DETAILED DESCRIPTION

The subject matter described herein is directed to chemically modified chondroitin sulfate (CS) NO-releasing polymer compounds with multi-action wound healing properties. As described herein, the polymer compounds can release 0.2-0.9 μmol NO mg⁻¹ compound in simulated wound fluid with NO-release half lives ranging from 20-110 min. The CS polymer compounds, functionalized with alkylamines, exhibit broad-spectrum bactericidal action against three strains each of Pseudomonas aeruginosa and Staphylococcus aureus ranging in antibiotic resistance profile. In particular, the functionalized CS NO-releasing polymer compounds described herein show several benefits compared to other glycosaminoglycan biopolymers as a pro-wound healing NO donor scaffold. These benefits include accelerated would closure and decreased bacterial burden, which are attributable to both active NO release and the CS biopolymer backbone.

Nitric oxide, an endogenous signaling molecule, may play an important therapeutic role in treating chronic wounds due to its innate roles in mitigating both inflammation and infection.¹⁶⁻¹⁹ Nitric oxide possesses broad-spectrum antibacterial action through multiple mechanisms (i.e., nitrosative and oxidative stresses), in which reactive byproducts of NO cause thiol nitrosation, DNA deamination, and destruction of cell membranes through lipid peroxidation.^{16,20,21} With NO's ability to readily penetrate through the EPS matrix, significantly lower concentrations of NO are required relative to traditional antibiotics to disrupt and eradicate biofilm-based bacteria.²²⁻²⁴ Bacterial resistance to NO is also unlikely due to these multiple mechanisms of antibacterial action.^{25,26} In addition to its bactericidal activity, endogenous NO is directly involved in the wound healing pathway.^{7,27} Nitric oxide is produced in large quantities (nM-μM) by immune cells (e.g., macrophages, neutrophils) as part of the immune response in the wound environment.^{24,28,29} Lesser concentrations of NO (pM-nM) are constitutively produced by endothelial cells and facilitate angiogenesis, collagen synthesis, and inflammatory cell proliferation and function.³⁻³³ These endogenous mechanisms have motivated the use of exogenous NO as a strategy for eradicating bacterial infections and assisting wound healing.

Treatment with gaseous NO has been demonstrated to reduce bacterial loads and accelerate wound closure.^{34,35} However, such treatment is limited to use in hospital settings where high pressure cylinders and oversight are available.^{28,36,37} N-diazeniumdiolate NO donors provide an alternative method to exogenous NO due to their spontaneous degradation to NO in physiological milieu.^{38,39} Low molecular weight N-diazeniumdiolate NO donors have been utilized as wound therapeutics and shown to accelerate wound closure and decrease bacterial burden in rodent models.^{40,41} While representing an efficacious system, the untargeted delivery of these small molecule NO donors and cytotoxicity of regenerated donor amines make such systems difficult to use as therapeutics.^{20,42} The covalent attachment of these NO donor functional groups to macromolecules, such as silica nanoparticles and biopolymers, mitigates these concerns.^{20,42} Macromolecular NO-release vehicles are easily incorporated within conventional wound therapies and dressings (e.g., fibrous bandages, hydrogels) to provide topical NO delivery.^{43,44} For example, Lowe et al. covalently bound NO donors to acrylonitrile-based terpolymers, which were then electrospun to form non-woven fibrous wound dressings.⁴³ In a different approach, Masters et al. reported on the covalent attachment of NO donors to poly(vinyl alcohol) hydrogels.⁴⁴ Treatment of wounds with NO derived from macromolecular scaffolds, such as those reported by Lowe et al. and Masters et al., has accelerated wound closure, eradicated wound pathogens, increased collagen deposition, and promoted angiogenesis.^{7,22,43,44}

Hyaluronic acid (HA) and chondroitin sulfate (CS), two biopolymers within the glycosaminoglycan (GAG) family, represent promising NO delivery agents due to their endogenous production and roles in inflammation and tissue repair.⁴⁵⁻⁴⁹ As additional benefits, both biopolymers exhibit high water solubility, low toxicity, and biodegradability.^{47,49} Hyaluronic acid is composed of alternating D-glucuronic acid and N-acetyl-D-glucosamine residues and acts as a signaling molecule for wound healing with resulting actions dependent on HA molecular weight.^{46,47,50,51} High molecular weight HA (≥1 MDa) is found in healthy tissue and signals for tissue maintenance.^{46,52} As a tissue stress response, endogenous HA is enzymatically degraded by hyaluronidases to lower molecular weights (1-800 kDa), signaling for tissue repair actions, such as angiogenesis, the release of pro-inflammatory cytokines, and collagen deposition.^{46,47,50,52}

Due to HA's involvement in the wound healing pathway, several studies have investigated the supplementation of exogenous HA as a potential therapy. Both high molecular weight and low molecular weight HA wound therapies have demonstrated enhanced wound healing through a number of outcomes, including improving skin mechanical properties, alleviating inflammation, and increasing wound closure rates, angiogenesis, collagen deposition, and wound moisture.^{47,53-57} Chondroitin sulfate possesses a similar structure to HA, with alternating D-glucuronic acid and N-acetyl-D-galactosamine residues and a sulfate group.^{58,59} In animal tissue, this sulfate group is found at either the 4 (chondroitin sulfate A; CSA) or 6 position (chondroitin sulfate C; CSC) of the N-acetyl-D-galactosamine residue.^{48,58,60} The specific interactions of CS with bioactive molecules are a function of the sulfation degree and profile of the CS backbone.^{49,58} Previous literature has reported that CS isomers influence inflammation, fibroblast/keratinocyte adhesion, proliferation, migration, angiogenesis, and collagen deposition, all of which are potentially beneficial for a wound therapeutic.^{59,61-66} The structures of both HA and CS allow for facile chemical modification of the carboxylic acid groups, including covalent attachment of NO donors.^{49,67} Through combination of the beneficial wound healing properties of HA and CS with the multifaceted roles of NO, the multimodal macromolecular NO delivery system described herein was developed.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

### I. Definitions

As used herein, “and/or” refers to and encompasses any and all possible combinations of one or more of the associated listed items, as well as the lack of combinations when interpreted in the alternative (“or”).

As used herein, the term “about,” when referring to a measurable value such as an amount of a compound or agent of the current subject matter, dose, time, temperature, and the like, is meant to encompass variations of ±20%, ±10%, ±5%, ±1%, ±0.5%, or even ±0.1% of the specified amount.

As used herein, conditional language used herein, such as, among others, “can,” “could,” “might,” “may,” “e.g.,” and the like, unless specifically stated otherwise or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The term “effective amount,” as used herein, refers to that amount of a recited compound that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, an effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some embodiments, an improvement in a condition can be a reduction in infection. In some embodiments, an improvement can be reduction of bacterial load (e.g., bioburden) on a surface or in a subject. Actual dosage levels of active ingredients in an active composition of the presently disclosed subject matter can be varied so as to administer an amount of the active compound(s) that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The term “biopolymer” refers to a polymeric substance occurring in living organisms, including polynucleotides (e.g., DNA, RNA), polysaccharides (e.g., cellulose), proteins (e.g., polypeptides), glycopeptides, peptidoglycans, and the like.

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, curing the illness, etc.

The terms “disrupting” and “eradicating” refer to the ability of the presently disclosed structures to combat biofilms. The biofilms may be partially eradicated or disrupted, meaning that the cells no longer attach to one another or to a surface. The biofilm may be completely eradicated, meaning that the biofilm is no longer an interconnected, cohesive, or continuous network of cells to a substantial degree.

The terms “nitric oxide donor” or “NO donor” refer to species and/or molecules that donate, release and/or directly or indirectly transfer a nitric oxide species, and/or stimulate the endogenous production of nitric oxide in vivo and/or elevate endogenous levels of nitric oxide in vivo such that the biological activity of the nitric oxide species is expressed at the intended site of action.

The terms “nitric oxide releasing” or “nitric oxide donating” refer to species that donate, release and/or directly or indirectly transfer any one (or two or more) of the three redox forms of nitrogen monoxide (NO+, NO−, NO (e.g., ·NO)) and/or methods of donating, releasing and/or directly or indirectly transferring any one (or two or more) of the three redox forms of nitrogen monoxide (NO+, NO−, NO). In some embodiments, the nitric oxide releasing is accomplished such that the biological activity of the nitrogen monoxide species is expressed at the intended site of action.

The term “microbial infection” as used herein refers to bacterial, fungal, viral, yeast infections, as well other microorganisms, and combinations thereof.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects. The subject “in need of” the methods disclosed herein can be a subject that is experiencing a disease state and/or is anticipated to experience a disease state, and the methods and compositions of the invention are used for therapeutic and/or prophylactic treatment.

For the general chemical formulas provided herein, if no substituent is indicated, a person of ordinary skill in the art will appreciate that the substituent is hydrogen. A bond that is not connected to an atom, but is shown, indicates that the position of such substituent is variable. A jagged line, wavy line, two wavy lines drawn through a bond or at the end of a bond indicates that some additional structure is bonded to that position. For a great number of the additional monomers disclosed herein, but not explicitly shown in structures, it is understood by those in the art of polymers, that these monomers can be added to change the physical properties of the resultant polymeric materials even where the elemental analysis would not indicate such a distinction could be expected. Such physical properties include solubility, charge, stability, cross-linking, secondary and tertiary structure, and the like. Moreover, if no stereochemistry is indicated for compounds having one or more chiral centers, all enantiomers and diastereomers are included. Similarly, for a recitation of aliphatic or alkyl groups, all structural isomers thereof also are included. Unless otherwise stated, groups shown as A₁ through Aₙ and referred to herein as an alkyl group, in the general formulas provided herein are independently selected from alkyl or aliphatic groups, particularly alkyl having 20 or fewer carbon atoms, and even more typically lower alkyl having 10 or fewer atoms, such as methyl, ethyl, propyl, isopropyl, and butyl. The alkyl may be optionally substituted (e.g., substituted or not substituted, as disclosed elsewhere herein). The alkyl may be a substituted alkyl group, such as alkyl halide (e.g. CX₃ where X is a halide, and combinations thereof, either in the chain or bonded thereto), alcohols (i.e. aliphatic or alkyl hydroxyl, particularly lower alkyl hydroxyl) or other similarly substituted moieties such as amino-, amino acid-, aryl-, alkyl aryl-, alkyl ester-, ether-, keto-, nitro-, sulfhydryl-, sulfonyl-, sulfoxide modified-alkyl groups.

The term “amino” and “amine” refer to nitrogen-containing groups such as NR₃, NH₃, NHR₂, and NH₂R, wherein R can be as described elsewhere herein. Thus, “amino” as used herein can refer to a primary amine, a secondary amine, or a tertiary amine. In some embodiments, one R of an amino group can be a diazeniumdiolate (i.e., NONO).

Whenever a group is described as being “optionally substituted” that group may be unsubstituted or substituted with one or more of the indicated substituents. Likewise, when a group is described as being “unsubstituted or substituted” (or “substituted or unsubstituted”) if substituted, the substituent(s) may be selected from one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group, a di-substituted amine group, a mono-substituted amine(alkyl), a di-substituted amine(alkyl), a diamino-group, a polyamino, a diether-group, and a polyether-group.

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” or “C₁-C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

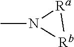

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. An alkyl group may be substituted or unsubstituted. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

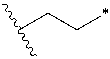 [SMILES: *CC*]

to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 6 carbon atoms. An alkylene group may be substituted or unsubstituted. For example, a lower alkylene group can be substituted by replacing one or more hydrogens of the lower alkylene group and/or by substituting both hydrogens on the same carbon with a C₃₋₆ monocyclic cycloalkyl group

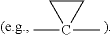

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic (such as bicyclic) hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers to a mono- or multi-cyclic (such as bicyclic) hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged, or spiro fashion. A cycloalkenyl group may be unsubstituted or substituted.

As used herein, “aryl” refers to a carbocyclic (all carbon) monocyclic or multicyclic (such as bicyclic) aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted. As used herein, “heteroaryl” refers to a monocyclic or multicyclic (such as bicyclic) aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, a “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

The terms “amino” and “unsubstituted amino” as used herein refer to a —NH₂ group.

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

When a range of integers is given, the range includes any number falling within the range and the numbers defining ends of the range. For example, when the terms “integer from 1 to 20” is used, the integers included in the range are 1, 2, 3, 4, 5, 6, 7, 8, 9, 10, etc., up to and including 20.

### Nitric Oxide Releasing Polymer Compounds

Nitric oxide, an endogenously produced diatomic free radical, is associated with numerous biological processes, including platelet aggregation and adhesion, vasodilation, wound repair, the immune response, the mediation of angiogenesis, and carcinogenesis. Deficiency of NO can lead to some degree of malfunction of NO-relevant physiological systems. Exogenous NO delivery may be an effective strategy for the resolution of biomedical therapies ranging from cardiovascular diseases, to antibacterial and anticancer therapies. NO delivery can also be used to achieve antimicrobial activity. However, the difficulty in delivering NO as a therapeutic warrants, in several embodiments, the use of assorted synthetic NO donors (e.g., N-diazeniumdiolates, S-nitrosothiols, metal nitrosyls, organic nitrates), in order to control NO delivery. N-diazeniumdiolates (NONOates) may be useful as NO donors because of their good stability and their capacity for proton-triggered NO delivery under physiological conditions. It has a relatively short biological half-life (seconds) and is reactive in nature. In several embodiments disclosed herein, the NO donor comprises any one of the following nitric oxide releasing moieties:

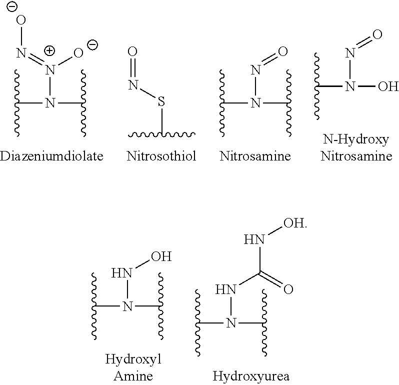

where “ [symbol] ” indicates attachment to other atoms within the structure (e.g., any instance of —H, —CH₂—, —CH—, etc.). In some embodiments, the NO donor is a N-diazeniumdiolate NO donor. In some embodiments, the NO donor is attached along a linear unit at a secondary amine as disclosed elsewhere herein.

The synthesis of scaffolds capable of controlled NO storage and release is important for taking advantage of NO's role in physiology and for developing NO-based therapeutics. In addition to the effects of NO disclosed above, NO is also a potent antibacterial agent that acts on bacteria via nitrosative and/or oxidative stress. NO is a broad-spectrum antibacterial agent and in some embodiments, scaffolds that deliver NO are capable of eradicating both bacteria and biofilms, primarily through the formation of reactive NO byproducts (e.g., peroxynitrite and dinitrogen trioxide) that cause oxidative and nitrosative damage to microbial DNA and/or membrane structures. Advantageously, the wide range of mechanisms by which NO exerts its antibacterial effects reduces the risk that bacteria will develop resistance. Thus, NO-releasing materials may be good targets to battle bacterial infection. The antibacterial efficacy of NO-releasing materials may be dependent on both NO payloads and associated release kinetics. In some instances, high NO total is an important parameter to effectively evaluate storage capability of good scaffolds. Additionally, in several embodiments disclosed herein, a high density of secondary amine groups imbues certain donors with a high NO storage capacity. However, NO release that is too fast and high NO storage may result in undesired toxicity to mammalian cells. Therefore, challenges exist in preparing biocompatible NO-releasing materials with high NO storage and low cytotoxicity, and such challenges, among others, are addressed according to several embodiments disclosed herein.

Several embodiments disclosed herein have one or more of the following advantages: efficient and unique synthesis routes and resultant chemical composition of polymer constructs. Controllable amounts of secondary-amines and diverse exterior terminal groups (e.g., hydroxyl, methyl, hydroxymethyl, and primary amine) can be provided. The NO storage and NO-release kinetics of the generated nitric-oxide releasing scaffolds can be tuned for a particular application. This tuning is achieved, in several embodiments, by altering the type and/or number of functionalized monomers of the formulae disclosed herein. In several embodiments, additional functionalization of the amines in the generated nitric-oxide releasing scaffolds, for example, by compounds with different compositions, further enables the control over NO-release kinetics. In some embodiments, the secondary amine group directly influences the stability of the N-diazeniumdiolate (or other NO carrier group), allowing for control over both NO storage and release kinetics.

As disclosed elsewhere herein, nitric oxide not only plays fundamental roles in several important biological processes, but also exhibits function as an antibacterial or anticancer agent. As disclosed elsewhere herein, various NO donors (e.g., N-diazeniumdiolates, S-nitrosothiols, metal nitrosyls, organic nitrates) can be used for controlled exogenous NO delivery. N-bound diazeniumdiolates are attractive because of their good stability and facile storage, which spontaneously undergo proton-triggered dissociation under physiological condition to regenerate the NO radicals. In several embodiments, progress has been made in preparing and testing biocompatible N-diazeniumdiolate-modified scaffolds, including those derived from chondroitin sulfate (CS).

Unlike current treatments, NO, an endogenously produced free radical, eradicates bacteria using a variety of mechanisms, including, but not limited to, lipid peroxidation, nitrosation of membrane proteins, and DNA damage via reactive oxygen/nitrogen species (e.g., peroxynitrite, dinitrogen trioxide). Multiple biocidal mechanisms allow NO to significantly diminish the risk of fostering bacterial resistance. Furthermore, NO has the improved ability to actively degrade both the biofilm matrix and mucus structure, thus allowing for more efficient biocidal action and mucociliary clearance.

As disclosed elsewhere herein, some embodiments disclosed herein pertain to the use of polymer scaffolds to deliver NO to achieve microbicidal activity. In some embodiments, the polymer scaffold is derived from a biopolymer. In some embodiments, the scaffold and/or biopolymer is water soluble. In some embodiments, the scaffold and/or biopolymer is and/or is biodegradable.

In several embodiments, the scaffolds, polymers, mixtures of polymers, etc., have structural units (e.g., repeat units, etc.) along a chain of a polymer.

In certain embodiments, the subject matter described herein is directed to a NO-releasing polymer compound comprising a unit structure of Formula I.

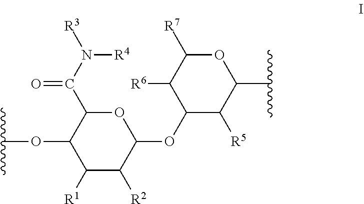

wherein,
R¹, R², and R⁵ are each independently selected from the group consisting of —OH, —NH₂, —CH₂OH, —C(O)OH, —NHC(O)—CH₃, C₁-C₆ alkoxy, —O—((CH₂)ₐO)_{b}—(CH₂)_{c}H, —NH—((CH₂)_{d}NH)ₑ—(CH₂)_{f}H, —X¹—((CH₂)_{g}X²)ₕ—(CH₂)ᵢH, —CH₂C(O)—X¹—((CH₂)_{g}X²)ₕ((CH₂)ⱼX³)ₖ—(CH₂)ᵢH, —X¹—((CH₂)_{g}X²)ₕ((CH₂)ⱼX³)ₖ—(CH₂)H, —X¹—(CH₂)_{g}X⁴, —CH₂C(O)—X¹—((CH₂)_{g}X²)ₕ(CH₂)ⱼX⁵, and —X¹—((CH₂)_{g}X²)ₕ(CH₂)ⱼX⁶;
R⁶ and R⁷ are each independently selected from the group consisting of —OH, —NH₂, —CH₂OH, —C(O)OH, C₁-C₆ alkoxy, —CH₂OSO₃, and —OSO₃, provided that at least one of R₆ and R₇ is —CH₂OSO₃⁻ or —OSO₃;
R³ is selected from the group consisting of —((CH₂)_{g}X²)ₕ—(CH₂)ᵢH, —((CH₂)_{g}X²)ₕ((CH₂)ⱼX³)ₖ—(CH₂)H, —(CH₂)_{g}X⁴, and —((CH₂)_{g}X²)ₕ(CH₂)ⱼX⁵;
R⁴ is selected from the group consisting of hydrogen and C₁-C₆ alkyl;
   a, b, c, d, e, f, g, h, i, j, k, and l are each independently an integer between 0 and 10;
   X¹, X², and X³ are each independently selected from the group consisting of —O—, —S—, —NH, —C(O)NH—, and a first NO donating moiety selected from the group consisting of

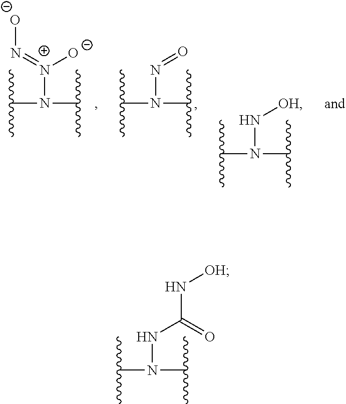 [SMILES: *N(*)/[N+]([O-])=N/[O-] ; *N(*)N=O ; *N(*)NO ; *N(*)NC(=O)NO]

X⁴, X⁵, and X⁶ are each independently selected from a second NO donating moiety selected from the group consisting of

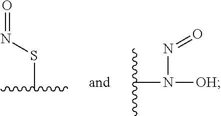

provided that the NO-releasing polymer compound contains at least one first NO donating moiety or second NO donating moiety.

Useful variables for R⁶ and R⁷ include those where one of R⁶ and R⁷ is —CH₂OSO₃⁻ or —OSO₃⁻ and the other is —CH₂OH or —OH. In certain embodiments, useful variables for R⁶ and R⁷ include those where R⁶ is —OSO₃⁻ and R⁷ is —CH₂OH. In certain embodiments, useful variables for R⁶ and R⁷ include those where R⁷ is —CH₂OSO₃⁻ and R⁶ is —OH.

Useful variables for R¹ and R² include —OH, —NH₂, —CH₂OH, —O—((CH₂)ₐO)_{b}—(CH₂)_{c}H, and —NH—((CH₂)_{d}NH)ₑ—(CH₂)_{f}H. In certain embodiments, R¹ and R² are each independently —OH.

Useful variables for R⁵ include —NHC(O)—CH₃.

Useful variables for R⁴ include hydrogen.

In certain embodiments of the above embodiment, the NO-releasing polymer compound contains the first NO-donating moiety

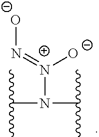 [SMILES: *N(*)/[N+]([O-])=N/[O-]]

Useful variables for R³ include —((CH₂)_{g}X²)ₕ((CH₂)ⱼX³)ₖ—(CH₂)ₗH. In certain embodiments of this embodiment, R³ is —((CH₂)_{g}X²)ₕ((CH₂)ⱼX³)ₖ—(CH₂)H, wherein g is 2; X² is

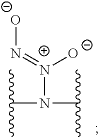 [SMILES: *N(*)/[N+]([O-])=N/[O-]]

h is 1; j is 2; X³ is O; and 1 is 0. In certain embodiments of this embodiment, R³ is —((CH₂)_{g}X²)ₕ((CH₂)ⱼX³)ₖ—(CH₂)H, wherein g is 3; X² is

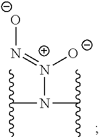 [SMILES: *N(*)/[N+]([O-])=N/[O-]]

h is 1; j is 3; X³ is NH; and 1 is 0. In certain embodiments of this embodiment, R³ is —((CH₂)_{g}X²)ₕ((CH₂)ⱼX³)ₖ—(CH₂)ᵢH, wherein g is 2; X² is

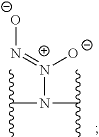 [SMILES: *N(*)/[N+]([O-])=N/[O-]]

h is 1; j is 2; X³ is NH; and 1 is 0. In certain other embodiments useful variables for R³ include

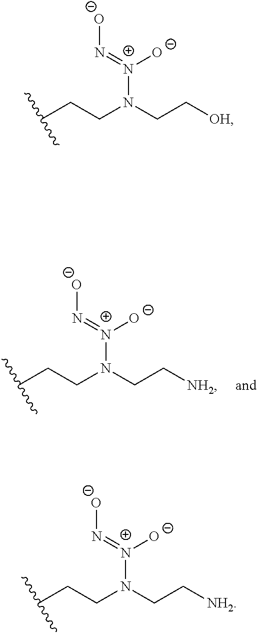

In certain embodiments of the above NO-releasing polymer compound, the compound has a total releasable NO storage in a range of 0.1-1.0 μmol of NO per mg of compound (measured in 10 nM PBS at pH 7.4 at 37° C.).

In certain embodiments of the above NO-releasing polymer compound, the compound has a NO half-life in the range of 0.1-24 hours (measured in 10 nM PBS at pH 7.4 at 37° C.).

In certain embodiments of the above NO-releasing polymer compound, the compound has a total releasable NO storage in a range of 0.1-1.0 μmol of NO per mg of compound as measured in simulated wound fluid (10% v/v FBS in PBS at 37° C.). In certain embodiments, the compound has a total releasable NO storage in a range of 0.2-0.9 μmol of NO per mg of compound as measured in simulated wound fluid (10% v/v FBS in PBS at 37° C.). In certain other embodiments, the compound has a total releasable NO storage of about 0.1, 0.2, 0.3, 0.4, 0.5, 0.6, 0.7, 0.8, 0.9, or 0.1 μmol of NO per mg of compound as measured in simulated wound fluid (10% v/v FBS in PBS at 37° C.).

In certain embodiments of the above NO-releasing polymer compound, the compound has a NO half-life in the range of 0.1-24 hours as measured in simulated wound fluid (10% v/v FBS in PBS at 37° C.). In certain embodiments of the above NO-releasing polymer compound, the compound has a NO half-life in the range of 0.3-3 hours as measured in simulated wound fluid (10% v/v FBS in PBS at 37° C.). In certain other embodiments, the compound has a NO half-life of about 0.1, 0.2, 0.3, 0.4, 0.5, 0.6, 0.7, 0.8, 0.9, 1.0, 1.1, 1.2, 1.3. 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 6.0, 7.0, 8.0, 9.0, or 10.0 hours as measured in simulated wound fluid (10% v/v FBS in PBS at 37° C.).

### Properties

In several embodiments, various structural units (e.g., repeat units), functionalization of structural units (with various moieties), levels of crosslinking (if crosslinked), molecular weight, concentrations, or other chemical features of the disclosed scaffolds contribute to the tunability of the properties of the scaffolds disclosed herein. In several embodiments, by changing one or more of these features, one or more properties of the scaffolds can be tuned. In several embodiments, the NO release rate, antimicrobial effect, water solubility, degradation rate, viscosity, gel firmness (where the scaffold forms a gel), viscoelasticity, modulus, etc. are tunable.

In several embodiments, properties of the polymer and or composition prepared therefrom can be tuned by adjusting the molecular weight of the polymer used. In several embodiments, the weight-average molecular weight (Mw) in kDa of polymers disclosed herein are greater than or equal to about: 2.5, 5.0, 7.0, 10, 15, 30, 50, 100, 200, 500, 750, 1,000, 2,000, 10,000, or ranges including and/or spanning the aforementioned values. In several embodiments, the number-average molecular weight (M) in kDa of polymers disclosed herein are greater than or equal to about: 2.5, 5.0, 7.0, 10, 15, 30, 50, 90, 100, 200, 500, 700, 1,000, 2,000, 10,000, or ranges including and/or spanning the aforementioned values. In several embodiments, the polymers disclosed herein may have n repeat units. In several embodiments, n equal to or at least about: 10, 25, 50, 100, 250, 500, 1000, 2500, 5000, 10000, or ranges including and/or spanning the aforementioned values. In several embodiments, size exclusion chromatography (SEC) can be used to measure the molecular weight of the scaffold structures disclosed herein. In several embodiments, multi-angle light scattering (SEC-MALS) detectors can be used. In several embodiments, the scaffold structures can be characterized using their polydispersity index. The polydispersity index (PDI) is a measure of the distribution of molecular mass in a given polymer sample. PDI can be calculated by dividing the weight average molecular weight and the number average molecular weight. In several embodiments, the scaffold structures have a PDI of greater than or equal to about: 1.05, 1.1, 1.2, 1.3, 1.5, 1.7, 1.8, 1.9, 2.0, or ranges including and/or spanning the aforementioned values.

In several embodiments, the polymers (or mixtures of polymers) may be water soluble and/or mutually miscible. In several embodiments, the scaffolds are soluble in water (at about 20° C.) at a concentration of greater than or equal to about: 1 mg/ml, 10 mg/ml, 20 mg/ml, 50 mg/ml, 100 mg/ml, 200 mg/ml, 300 mg/ml, 400 mg/ml, 500 mg/ml, or ranges including and/or spanning the aforementioned values.

According to several embodiments, different NO carrying polymers can be combined to prepare aqueous solutions comprising concentrations equal to or at least about: 100 μg/mL, and can be higher, e.g. about 1 mg/ml, about 5 mg/ml, about 10 mg/ml, about 20/ml, or about 40 mg/ml or higher. The amount of the second polymer in the aqueous composition can be at least about 10% by weight, based on the weight of the first polymer, and may be higher, e.g., at least about 20% by weight, at least about 30% by weight, or at least about 50% by weight, same basis. The polymers in an aqueous composition are selected such the polymers are mutually miscible. As noted above, the first polymer with antimicrobial activity and the second polymer with antimicrobial activity are considered mutually miscible if at least about 90% of the polymeric components remain mutually soluble 24 hours after mixing and maintaining at room temperature in water at a concentration of each polymer of 1 mg/ml, upon visible examination. Surprisingly, such mutual miscibility of the water polymers can be achieved, despite an expectation of phase separation due to the typical mutual incompatibility of polymers in aqueous solution at the 1 mg/ml concentrations and molecular weights described herein. The aqueous compositions described herein can be prepared by intermixing the individual polymeric components with water, e.g., at room temperature with stirring.

In several embodiments, the polymers (or mixtures of polymers, etc.) disclosed herein have properties characteristic of a viscous fluid and/or of a gel. In several embodiments, the polymers (or mixtures of polymers, etc.) have a gelling point at room temperature (in water or PBS) at a concentration (in w/w %) of less than or equal to about: 0.5%, 1%, 2.5%, 5%, 10%, or ranges including and/or spanning the aforementioned values. In several embodiments, the polymers (or mixtures of polymers, etc.) may have a gelling point in water. In several embodiments, the polymers gel in water (at about 20° C.) at a concentration of greater than or equal to about: 0.5 mg/ml, 1 mg/ml, 10 mg/ml, 20 mg/ml, 50 mg/ml, 100 mg/ml, 250 mg/ml, or ranges including and/or spanning the aforementioned values. In several embodiments, at a concentration of 5% w/w solution, the polymers have a viscosity (in cPa·s at 20° C.) of equal to or at least about: 10, 50, 100, 1,000, 2,000, 5,000, 10,000, or ranges including and/or spanning the aforementioned values. In several embodiments, the polymers have an intrinsic viscosity of equal to or greater than about: 0.5 m³/kg, 1.0 m³/kg, 2.0 m³/kg, 4.0 m³/kg, 8.0 m³/kg, or ranges including and/or spanning the aforementioned values.

In several embodiments, at a concentration of 5% w/w solution, the polymers have a firmness of equal to or at least about: 1.0 mN, 2.5 mN, 5 mN, 10 mN, 15 mN, 20 mN, 30 mN, 50 mN, or ranges including and/or spanning the aforementioned values. In several embodiments, at a concentration of 5% w/w solution, the polymers have a work of adhesion (in mN*mm) of equal to or at least about: 1.0, 2.5, 5, 10, 15, 20, 30, 50, 100, or ranges including and/or spanning the aforementioned values. In several embodiments, at a concentration of 5% w/w solution, the polymers have a storage modulus (G′) in Pa of equal to or at least about: 250, 500, 1,000, 2,000, 4,000, 5,000, 10,000, or ranges including and/or spanning the aforementioned values. In several embodiments, at a concentration of 5% w/w solution, the polymers have an elastic modulus (G″) in Pa of equal to or at least about: 25, 50, 100, 200, 400, 500, 1,000, 2,000, 5,000, 10,000, or ranges including and/or spanning the aforementioned values. In several embodiments, the aqueous composition is characterized by a barrier activity, as measured by a decrease in the diffusion rate of an anionic dye of more than 2 logs at a total scaffold concentration of 40 mg/ml or less.

In several embodiments, the gels are stable at a variety of temperatures 20° C. (e.g., 40° C., 45° C., 55° C., 60° C., 80° C., etc.) and are stable for prolonged storage periods (e.g., 10 hours, 20 hours, 22 hours, 25 hours, 30 hours, etc., days such as 1 day, 3 days, 5 days, 6 days, 7 days, 15 days, 30 days, 45 days, etc., weeks such as 1 week, 2 weeks, 3 weeks, 4 weeks, 6 weeks, 8 weeks, etc., months such as 1 month, 2 months, 3 months, 4 months, 5 months, 6 months, etc., or even years (1 year or greater)).

In several embodiments, the viscosity of the composition increases with increasing temperature, as described above. In several embodiments, the viscosity of the composition decreases with decreasing temperature. For example, if the composition is above the gelling temperature, then the composition has a relatively high viscosity, such as in the form of a gel. In several embodiments, if the composition is cooled to below the gelling temperature, then the composition decreases in viscosity, such as in the form of a liquid. In several embodiments, as such, the polymers as disclosed herein may be reversible polymers (e.g., thermoreversible polymers), where the transition from liquid to gel may be reversed upon exposure to appropriate conditions. For instance, as described above, compositions of the present disclosure include thermoreversible polymers, where the viscosity of the composition may be changed depending on the temperature of the composition. In several embodiments, the tunability of the viscosity enables a tailored composition profile upon delivery (e.g., more liquid at a delivery temperature and more viscous at, for example, body temperature).

In several embodiments, the polymers are characterized by a degree of swelling when exposed to water. In some embodiments, the swelling degree % of the polymers disclosed herein is equal to or at least about: 100, 250, 500, 1,000, 2,000, 5,000, or ranges including and/or spanning the aforementioned values. In other words, the polymers may swell or otherwise expand by 2×, 4×, 5×, 10×, 20×, 50×, 100×, or more.

In certain embodiments, the polymers disclosed herein have a gelling temperature similar to the normal body temperature of a subject, such as similar to human body temperature, or 37° C. By gelling temperature is meant the point on intersection between the plot for the elastic modulus and the plot for the viscous modulus. In some cases, if the composition is below the gelling temperature, then the composition has a relatively low viscosity, such as in the form of a liquid. In some instances, if the composition is above the gelling temperature, then the composition increases in viscosity (e.g., polymerizes), such that the composition is in the form of a gel. Compositions that transition from a liquid to a gel may facilitate administration of the composition to the subject, for example by facilitating injection of a low viscosity (e.g., liquid) composition at a temperature below the gelling temperature. After injection of the composition to the target treatment site, the temperature of the composition may increase due to absorption of heat from the surrounding body tissue, such that the composition increases in viscosity (e.g., transitions from a liquid to a gel, or polymerizes), thus providing structural and/or geometric support to the body tissue at the target treatment site. In some instances, gelling of the composition at the target treatment site may also facilitate retention of the composition at the treatment site by reducing the diffusion and/or migration of the composition away from the treatment site. In certain embodiments, the composition has a gelling temperature of 30° C. to 40° C., such as from 32° C. to 40° C., including from 35° C. to 40° C. In certain instances, the composition has a gelling temperature of 37° C.

In some embodiments, the methods disclosed herein provide NO-releasing polymers having NO storage capacities (in μmol NO/mg polymers) of greater than or equal to about: 0.25, 0.4, 0.5, 1.0, 1.5, 2.0, 3.0, or ranges including and/or spanning the aforementioned values. In some embodiments, within 2 h of being added to a PBS buffer solution as described in the Examples, the NO-releasing polymers, release greater than or equal to about: 25%, 50%, 75%, 85%, 90%, 95%, 100%, or ranges including and/or spanning the aforementioned values, their total wt % of bound NO.

In some embodiments, the NO release may occur over a period of about 0.01 hours, 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, 60 hours, or ranges including and/or spanning the aforementioned values. In several embodiments, the NO release half-life is equal to or at least about: 0.01 hours, 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, 60 hours, or ranges including and/or spanning the aforementioned values. In some embodiments, the NO release occurs in less than or equal to about: 0.01 hours, 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, 60 hours, or ranges including and/or spanning the aforementioned values. In some embodiments, nitrosamine is not present during NO release. As used herein the phrase “nitrosamine is not present” refers to levels of nitrosamine which are not detectable as determined by a UV-vis spectrum (or by other accepted methods in the art).

In some embodiments, the disclosed scaffolds and/or polymers of the disclosed compositions have a degradation rate per hour in an amylase enzyme exposure assay of less than or equal to about: 0.2%, 0.5%, 1.0%, 1.5%, 2.5%, 5.0%, 10%, or ranges including and/or spanning the aforementioned values.

In some embodiments, the disclosed functionalized NO-releasing polymers have antimicrobial activity. In some embodiments, the disclosed functionalized NO-releasing polymers provide greater than or equal to 90% bacterial reduction in a bacterial viability assay performed under static conditions over 2 hours against one or more of P. aeruginosa and/or S. aureus at a polymer concentration of equal to or less than about: 16 mg/ml, 12 mg/ml, 10 mg/ml, 8 mg/ml, 6 mg/ml, 4 mg/ml, 2 mg/ml, 1 mg/ml, 0.5 mg/ml, or ranges including and/or spanning the aforementioned values. In some embodiments, the disclosed functionalized NO-releasing polymers provide greater than or equal to 99% bacterial reduction and/or a 2 to 3 log reduction in a bacterial viability assay performed under static conditions over 2 hours against a gram positive bacteria at a polymer concentration of equal to or less than about: 16 mg/ml, 12 mg/ml, 10 mg/ml, 8 mg/ml, 6 mg/ml, 4 mg/ml, 2 mg/ml, 1 mg/ml, 0.5 mg/ml, or ranges including and/or spanning the aforementioned values. In some embodiments, the disclosed functionalized NO-releasing polymers provide greater than or equal to 99% bacterial reduction and/or a 2 to 3 log reduction in a bacterial viability assay performed under static conditions over 2 hours against a gram negative bacteria at a polymer concentration of equal to or less than about: 8 mg/ml, 6 mg/ml, 4 mg/ml, 2 mg/ml, 1 mg/ml, 0.5 mg/ml, or ranges including and/or spanning the aforementioned values. In several embodiments, bacterial reduction is greater than 95%, greater than 98%, or greater than 99%.

### Compositions

In several embodiments, the polymers are administered as aqueous gels, e.g., topically. In several embodiments, the gels comprise one or more salts and are isotonic. In several embodiments, compositions can take the form of, for example, tablets or capsules prepared by a conventional technique with pharmaceutically acceptable excipients, such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art. For example, a therapeutic agent can be formulated in combination with hydrochlorothiazide, and as a pH stabilized core having an enteric or delayed release coating which protects the therapeutic agent until it reaches the target organ.

In several embodiments, the composition includes two or more polymers with a certain ratio (w/w). In some cases, the ratio (w/w) is 1:10, or 1:9, or 1:8, or 1:7, or 1:6, or 1:5, or 1:4, or 1:3, or 1:2, or 1:1, or 2:1, or 3:1, or 4:1, or 5:1, or 6:1, or 7:1, or 8:1, or 9:1, or 10:1. For example, the ratio (w/w) may range from 1:1 to 10:1, such as 2:1 to 10:1, including 3:1 to 10:1, or 4:1 to 10:1, or 4:1 to 9:1, or 4:1 to 8:1, or 4:1 to 7:1, or 4:1 to 6:1. In certain embodiments, the ratio (w/w) is 5:1. These polymers can then be dissolved mutually in aqueous solution to provide a gel or solution. In several embodiments, each polymer of the mixture is provided at a concentration of less than or equal to about: 1 mg/ml, 10 mg/ml, 20 mg/ml, 50 mg/ml, 100 mg/ml, 250 mg/ml, or ranges including and/or spanning the aforementioned values.

### METHODS OF USE

In certain embodiments, the subject matter described herein is directed to a method for treating a tissue defect comprising positioning any of the polymers described herein at, over, or into the tissue defect. In several embodiments, the tissue defect is a wound. Several embodiments provide a method for treating a wound, for performing tissue repair, and/or for providing tissue and organ supplementation. In several embodiments, the first step of treating a tissue defect, wound, and/or supplementing and replacing tissue involves identifying a patient in need of an antimicrobial scaffold to aid in the remedying and healing of a tissue defect, healing of a wound, or in need of a tissue supplement. In certain embodiments, the wound is infected with a bacterial pathogen.

A non-limiting list of patients in need of an antimicrobial scaffold includes patients suffering tissue defects. In several embodiments, the patients in need of an antimicrobial scaffold suffer from wounds including those from burns, skin ulcers, lacerations, bullet holes, animal bites, and other wounds prone to infection. Antimicrobial polymers can also be used in the treatment of diabetic foot ulcers, venous leg ulcers, pressure ulcers, amputation sites, in other skin trauma, or in the treatment of other wounds or ailments. Patients in need of an antimicrobial scaffold also include patients in need of repair and supplementation of tendons, ligaments, fascia, and dura mater. Degradable antimicrobial polymers can be used in supplement tissue in procedures including, but not limited to, rotator cuff repair, Achilles tendon repair, leg or arm tendon or ligament repair (e.g., torn ACL), vaginal prolapse repair, bladder slings for urinary incontinence, breast reconstruction following surgery, hernia repair, staple or suture line reinforcement, bariatric surgery repair, pelvic floor reconstruction, dural repair, gum repair, bone grafting, and reconstruction. Further, a patient in need of an antimicrobial scaffold also includes one in need of tissue or organ replacement. In several embodiments, the antimicrobial polymers described herein can be used as fillers and/or to supplement and/or replace tissue by acting as an artificial extracellular matrix. In such an application, an antimicrobial scaffold can be used to support cell and tissue growth. Briefly, cells can be taken from a patient or a viable host and seeded on an antimicrobial scaffold either in vivo or ex vivo. Then as the patient's natural tissues invade the material, it is tailored to degrade and leave only naturally occurring tissues and cells free of bacterial infection.

In several embodiments, applications also include delivery of therapeutic molecules to a localized site, use as adhesives or sealants, and as viscosupplements, and in wound healing, among others. The stabilized compositions may also be used as tissue fillers, dermal fillers, bone fillers, bulking agents, e.g., as a urethral or an esophageal bulking agent, and embolic agents as well as agents to repair cartilage defects/injuries and agents to enhance bone repair and/or growth. In several embodiments, an antimicrobial scaffold can be placed in or on a patient in, for example, a void space to fill the space.

In several embodiments, provided are polymers for repairing an injured tissue. In several embodiments, the composition is formulated for administration to a target treatment site in a subject. For example, the composition may be formulated to facilitate administration to a damaged or infected tissue in a subject.

In several embodiments, after administration of the composition (e.g., the antimicrobial scaffold), the composition may increase in temperature due to absorption of heat from surrounding body tissue of the subject. In several embodiments, the body temperature of the subject is sufficient to cause the composition to increase in viscosity (e.g., transition from a liquid to a gel. In several embodiments, the increase in viscosity (e.g., gelling) may give rise to a 3-dimensional network sufficient to provide structural and/or geometric support to a body tissue, such as a cardiac tissue (e.g., a cardiac tissue of an infarct region). In several embodiments, a syringe or catheter may be used to inject the composition in vivo. In several embodiments, the composition may be injected directly to the treatment site, or may be allowed to partially pre-heat in the syringe in order to increase the viscosity of the composition prior to injection. In several embodiments, a pre-heated formulation may reduce the possibility that a less viscous composition may diffuse and/or migrate away from the tissue area of interest after injection.

Dental caries (e.g., tooth decay) is another important disease state that affects 60%-70% school age children and the majority of adults in most industrialized countries. Worldwide, 11% of the total population suffers from severe periodontitis, which contributes to tooth loss and systematic diseases such as coronary, cardiovascular, stroke, and adverse pregnancy outcomes. Of >700 microorganisms in the oral cavity, cariogenic bacteria (e.g., Streptococcus mutans, Actinomyces viscosus) and periodontal pathogens (e.g., Porphyromonas gingivalis, Aggregatibacter actinomycetemcomitans) play a major role in the initiation and progression of oral diseases. Oral disease is among the most prevalent health problems faced by humans. Gram-positive cariogenic (e.g., Streptococcus mutans, Actinomyces viscosus) and Gram-negative periodontal (e.g., Porphyromonas gingivalis, Aggregatibacter actinomycetemcomitans) bacteria represent the main aggravators associated with the evolution and progression of dental caries and periodontal disease, respectively. Unfortunately, current treatments to combat these pathogens come with undesirable side effects. For example, the systemic use of antibiotics may result in gastrointestinal disturbance and foster bacterial resistance. Chlorhexidine, a common oral antiseptic, can alter taste, stain teeth and tongue, and irritate buccal mucosa. Macromolecule NO-delivering vehicles (e.g., silica nanoparticles, gold, etc.) kill Gram-negative periodontal pathogens. However, these materials have not been demonstrated to kill Gram-positive cariogenic bacteria at a safe concentration (e.g., a concentration that is bacteriocidal but non-toxic towards mammalian cells). Similar with those nanomaterials, the lack of biodegradability and potential cytotoxicity of the silica nanoparticles also hinders their future for biomedical application. Current research also focuses on utilizing nanomaterials including silver, gold, zinc, and copper, as replacement for traditional antibiotics that suffered from fostering bacterial resistance. However, these nanomaterials may accumulate inside the body and may cause accumulative toxicity, limiting their future for certain applications. Developing oral therapeutics that are capable of killing those disease-causing bacteria is important to maintain a healthy oral cavity. In several embodiments, the structures disclosed herein (e.g., NO scaffolds and/or polymers), resolve one or more of these issues or others.

In several embodiments, the compositions disclosed herein may be used as eye drop formulations (e.g., artificial tears). In several embodiments, the composition comprises from about 0.1% to about 1.0% of the scaffold (or at a concentration as disclosed elsewhere herein). In several embodiments, the mixture comprises more than one type of polymer scaffold with the second polymer scaffold being present in an amount of 0.05% to about 0.15% (or at a concentration as disclosed elsewhere herein).

Cystic fibrosis (CF) is a genetic disorder characterized by poor mucociliary clearance and chronic bacterial infections. As shown herein, in several embodiments, nitric oxide (NO) has broad spectrum antibacterial activity against CF-relevant bacteria, making it an attractive alternative to traditional antibiotics. Treatment with NO limits bacterial resistance due to its multiple biocidal mechanisms (e.g., induction of nitrosative and oxidative stress).

CF is a debilitating disease characterized by chronic bacterial infection of the lungs, resulting in life expectancies as low as two decades. A genetic defect in the CF transmembrane conductance regulator (CFTR) impedes the normal transport of ions (e.g., Cl⁻) to the airway surface liquid, inhibiting water transport. As such, the airway epithelium dehydrates, creating thickened mucus that can no longer be efficiently cleared via mucociliary clearance mechanisms. As goblet cells continually excrete mucins into the dehydrated airway, mucus accumulation is accelerated to the point where the cilia become damaged, or nonfunctional, and are unable to clear mucus from the airway. Planktonic bacteria thrive in this static environment, promoting the formation of complex communities of pathogenic bacteria known as biofilms. The exopolysaccharide matrix produced by these biofilms inhibits oxygen diffusion, creating pockets of anaerobic environments and altering bacterial metabolism. This combination of a concentrated mucus layer and robust biofilms severely decreases the antibacterial efficacy of common CF therapies.

In several embodiments, the microbial load to be reduced and/or eliminated comprises drug-resistant bacteria. In several embodiments, the drug-resistant bacteria comprise carbapenem-resistant Enterobacteriaceae. In several embodiments, the drug-resistant bacteria comprise Methicillin-resistant Staphylococcus aureus. In several embodiments, the microbe comprises human immunodeficiency virus, herpes simplex virus, papilloma virus, parainfluenza virus, influenza, hepatitis, Coxsackie Virus, herpes zoster, measles, mumps, rubella, rabies, pneumonia, (hemorrhagic viral fevers, H1N1, and the like), prions, parasites, fungi, mold, yeast and bacteria (both gram-positive and gram-negative) including, among others, Candida albicans, Aspergillus niger, Escherichia coli (E. coli), Pseudomonas aeruginosa (P. aeruginosa), and Staphylococcus aureus (S. aureus), Group A streptococci, S. pneumoniae, Mycobacterium tuberculosis, Campylobacter jejuni, Salmonella, Shigella, P. gingivalis, A. actinomycetemcomitans, A. viscosus, and/or S. mutans and a variety of drug resistant bacteria. The terms microorganism and microbe shall be used interchangeably. Microbes can include wild-type, genetically-engineered or modified organisms. In several embodiments, the formulations and methods disclosed herein are for topical use or treatment of a surface, such as the oral mucosa.

In some embodiments, the scaffolds and/or compositions thereof may be administered by direct injection or application to, for example, an injured tissue. Suitable routes also include injection or application to a site adjacent to the injured tissue. Administration may include parenteral administration (e.g., intravenous, intramuscular, or intraperitoneal injection), subcutaneous administration, administration into vascular spaces, and/or administration into joints (e.g., intra-articular injection). Additional routes of administration include intranasal, topical, vaginal, rectal, intrathecal, intraarterial, and intraocular routes. In several embodiments, the scaffolds and compositions disclosed herein can be applied as a gel to a site of treatment. In several embodiments, the scaffolds and compositions can be applied as a liquid.

In several embodiments, liquid preparations for oral or topical administration can take the form of, for example, solutions, syrups or suspensions, or they can be presented as a dry product for constitution with water or another suitable vehicle before use. Such liquid preparations can be prepared by conventional techniques with pharmaceutically acceptable additives, such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations also can contain buffer salts, flavoring, coloring and sweetening agents as appropriate. Preparations for oral administration can be suitably formulated to give controlled release of the active compound. For buccal administration the compositions can take the form of tablets or lozenges formulated in a conventional manner.

In several embodiments, the disclosed compounds also can be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds also can be formulated in rectal compositions (e.g., suppositories or retention enemas containing conventional suppository bases, such as cocoa butter or other glycerides), creams or lotions, or transdermal patches.

Pharmaceutical formulations also are provided which are suitable for administration as an aerosol by inhalation. In several embodiments, the polymer structures described herein are formulated in solution and/or aerosol form. In several embodiments, these formulations comprise a solution or suspension of a polymers described herein. In several embodiments, the desired formulation can be placed in a small chamber and nebulized. Nebulization can be accomplished by compressed air or by ultrasonic energy to form a plurality of liquid droplets or solid particles comprising the compounds. For example, the presently disclosed NO-releasing polymer compounds can be administered via inhalation to treat bacterial infections related to cystic fibrosis. Cystic fibrosis-related bacterial infections include, but are not limited to Stenotrophomonas, Mycobacterium avium intracellulaire and M. abcessus, Burkholderia cepacia and Pseudomonas aeruginosa (P. aeruginosa) infections.

In certain embodiments, the subject matter described herein is directed to a method of treating a wound infected with a bacterial pathogen, comprising contacting the wound with an effective amount of a NO-releasing polymer compounds described herein. In certain embodiments of this method, the bacterial pathogen is selected from the group consisting of P. aeruginosa and S. aureus.

The subject matter described herein is directed to the following embodiments:
1. A NO-releasing polymer compound comprising a unit structure of Formula I:

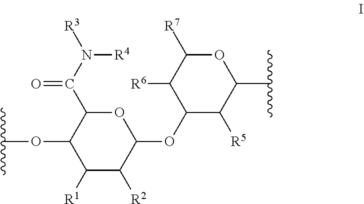

wherein,
   R¹, R², and R⁵ are each independently selected from the group consisting of —OH, —NH₂, —CH₂OH, —C(O)OH, —NHC(O)—CH₃, C₁-C₆ alkoxy, —O—((CH₂)ₐO)_{b}—(CH₂)_{c}H, —NH—((CH₂)_{d}NH)ₑ—(CH₂)_{f}H, —X¹—((CH₂)_{g}X²)ₕ—(CH₂)ᵢH, —CH₂C(O)—X¹—((CH₂)_{g}X²)ₕ((CH₂)ⱼX³)ₖ—(CH₂)ᵢH, —X¹—((CH₂)_{g}X²)ₕ((CH₂)ⱼX³)ₖ—(CH₂)ₗH, —X¹—(CH₂)_{g}X⁴, —CH₂C(O)—X¹—((CH₂)_{g}X²)ₕ(CH₂)ⱼX⁵, and —X¹—((CH₂)_{g}X²)ₕ(CH₂)ⱼX⁶;
   R⁶ and R⁷ are each independently selected from the group consisting of —OH, —NH₂, —CH₂OH, —C(O)OH, C₁-C₆ alkoxy, —CH₂OSO₃, and —OSO₃, provided that at least one of R₆ and R₇ is —CH₂OSO₃⁻ or —OSO₃;
   R³ is selected from the group consisting of —((CH₂)_{g}X²)ₕ—(CH₂)ᵢH, —((CH₂)_{g}X²)ₕ((CH₂)ⱼX³)ₖ—(CH₂)ₗH, —(CH₂)_{g}X⁴, and —((CH₂)_{g}X²)ₕ(CH₂)ⱼX⁵;
   R⁴ is selected from the group consisting of hydrogen and C₁-C₆ alkyl;
      a, b, c, d, e, f, g, h, i, j, k, and l are each independently an integer between 0 and 10;
      X¹, X², and X³ are each independently selected from the group consisting of —O—, —S—, —NH, —C(O)NH—, and a first NO donating moiety selected from the group consisting of

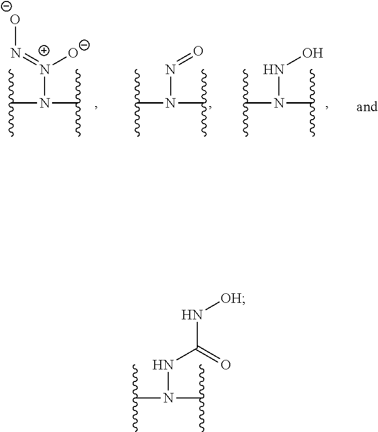

X⁴, X⁵, and X⁶ are each independently selected from a second NO donating moiety selected from the group consisting of

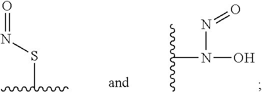

provided that the NO-releasing polymer compound contains at least one first NO donating moiety or second NO donating moiety.
2. The NO-releasing polymer compound of embodiment 1, wherein one of R⁶ and R⁷ is —CH₂OSO₃⁻ or —OSO₃⁻ and the other is —CH₂OH or —OH.
3. The NO-releasing polymer compound of embodiment 1 or 2, wherein R⁶ is —OSO₃⁻ and R⁷ is —CH₂OH.
4. The NO-releasing polymer compound of any one of embodiments 1-3, wherein R⁷ is —CH₂OSO₃⁻ and R⁶ is —OH.
5. The NO-releasing polymer compound of any one of embodiments 1-4, wherein R¹ and R² are each independently selected from the group consisting of —OH, —NH₂, —CH₂OH, —O—((CH₂)ₐO)_{b}—(CH₂)_{c}H, and —NH—((CH₂)_{d}NH)ₑ—(CH₂)_{f}H.
6. The NO-releasing polymer compound of embodiment 5, wherein R¹ and R² are each independently —OH.
7. The NO-releasing polymer compound of any one of embodiments 1-6, wherein R⁵ is —NHC(O)—CH₃.
8. The NO-releasing polymer compound of any one of embodiments 1-7, wherein R⁴ is hydrogen.
9. The NO-releasing polymer compound of any one of embodiments 1-8, wherein the NO-releasing polymer compound contains the first NO-donating moiety

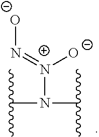 [SMILES: *N(*)/[N+]([O-])=N/[O-]]

10. The NO-releasing polymer compound of any one of embodiments 1-9, wherein R³ is —((CH₂)_{g}X²)ₕ((CH₂)ⱼX³)ₖ—(CH₂)ₗH.
11. The NO-releasing polymer compound of embodiment 10, wherein:
   g is 2;
   X² is

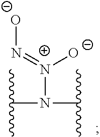 [SMILES: *N(*)/[N+]([O-])=N/[O-]]

h is 1;
j is 2;
X³ is O; and
l is 0.
12. The NO-releasing polymer compound of embodiment 10, wherein:
   g is 3;
   X² is

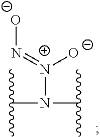 [SMILES: *N(*)/[N+]([O-])=N/[O-]]

h is 1;
j is 3;
X³ is NH; and
l is 0.
13. The NO-releasing polymer compound of embodiment 10, wherein:
   g is 2;
   X² is

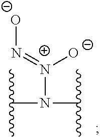 [SMILES: *N(*)/[N+]([O-])=N/[O-]]

h is 1;
j is 2;
X³ is NH; and
l is 0.
14. The NO-releasing polymer compound of any one of embodiments 1-13, wherein R³ is selected from the group consisting of

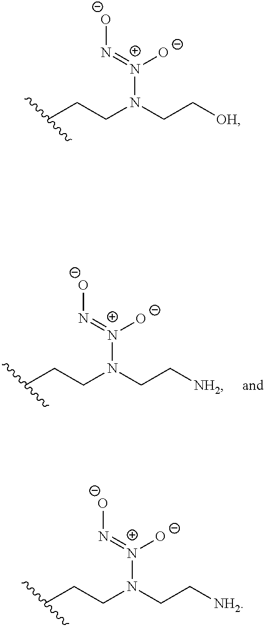

15. The NO-releasing polymer compound of embodiment 1, wherein the compound has a total releasable NO storage in a range of 0.1-1.0 μmol of NO per mg of compound (measured in 10 nM PBS at pH 7.4 at 37° C.).
16. The NO-releasing polymer compound of embodiment 1, wherein the compound has a NO half-life in the range of 0.1-24 hours (measured in 10 nM PBS at pH 7.4 at 37° C.).
17. The NO-releasing polymer compound of embodiment 1, wherein the compound has a total releasable NO storage in a range of 0.1-1.0 μmol of NO per mg of compound as measured in simulated wound fluid (10% v/v FBS in PBS at 37° C.).
18. The NO-releasing polymer compound of embodiment 17, wherein the compound has a total releasable NO storage in a range of 0.2-0.9 μmol of NO per mg of compound as measured in simulated wound fluid (10% v/v FBS in PBS at 37° C.).
19. The NO-releasing polymer compound of embodiment 1, wherein the compound has a NO half-life in the range of 0.1-24 hours as measured in simulated wound fluid (10% v/v FBS in PBS at 37° C.).
20. The NO-releasing polymer compound of embodiment 19, wherein the compound has a NO half-life in the range of 0.3-3 hours as measured in simulated wound fluid (10% v/v FBS in PBS at 37° C.).
21. A method of treating a wound infected with a bacterial pathogen, comprising contacting the wound with an effective amount of the NO-releasing polymer compound of any one of embodiments 1-20.
22. The method of embodiment 21, wherein the bacterial pathogen is selected from the group consisting of P. aeruginosa and S. aureus.

The following examples are offered by way of illustration and not by way of limitation.

### EXAMPLES

### Materials and Methods

Materials. Ultra-low (<6 kDa; HA6), super-low (<50 kDa; HA50), and extra-low (80-110 kDa; HA90) molecular weight hyaluronic acid were purchased from Lotioncrafter (Eastsound, WA). Chondroitin sulfate A sodium salt (average molecular weight 20-30 kDa) and chondroitin sulfate C sodium salt were purchased from Biosynth Carbosynth (Compton, United Kingdom). Bis(3-aminopropyl)amine (DPTA), diethylenetriamine (DETA), N-(2-hydroxyethyl)ethylenediamine (HEDA), 1-ethyl-3-(3-dimethylaminopropyl)carbodiimide hydrochloride (EDC), N-hydroxysuccinimide (NHS), phenazine methosulfate (PMS), bovine collagen solution (type I), lipopolysaccharides from Escherichia coli O111:B4 (LPS), antifoam B emulsion, and qPCR primers for Pseudomonas aeruginosa (PAO1) quantification were purchased from MilliporeSigma (St. Louis, MO). 3-(4,5-dimethylthizol-2-yl)-5-(3-carboxymethoxyphenyl)-2-(4-sulfophenyl)-2H-tetrazolium inner salt (MTS) was purchased from Biovision (Milpitas, CA). Trypsin, penicillin streptomycin (PS), fetal bovine serum (FBS), Dulbecco's modified Eagle's medium (DMEM), keratinocyte serum-free medium (SFM; with human recombinant epidermal growth factor, rEGF), bovine serum albumin (BSA), tumor necrosis factor recombinant human protein (TNF-α), and common laboratory salts and solvents were purchased from Thermo Fisher Scientific (Fair Lawn, NJ). DNeasy Blood and Tissue Kit was purchased from Qiagen (Hilden, Germany). iQ SYBR Green Supermix was purchased from Bio-Rad Laboratories, Inc. (Hercules, CA). HEK-BLU mTLR4 cells, HEK-BLUE Null1-v cells, HEK-BLUE Selection, NORMOCIN, ZEOCIN, and QUANTI-BLUE solutions were purchased from Invivogen (San Diego, CA). Tryptic soy broth (TSB), Mueller Hinton II broth (MHB), and tryptic soy agar (TSA) were obtained from Becton, Dickinson, and Company (Franklin Lakes, NJ). Human dermal fibroblasts (HDF) and human epidermal keratinocytes (HEK) were obtained from the UNC Tissue Culture Facility (Chapel Hill, NC). Pseudomonas aeruginosa (P. aeruginosa, PAO1; ATCC 47085), Staphylococcus aureus (S. aureus; ATCC 29213), multidrug-resistant P. aeruginosa (MDR-PA; ATCC BAA-2110), and methicillin-resistant S. aureus (MRSA; ATCC 33591) were obtained from the American Type Culture Collection (Manassas, VA). Clinical multidrug resistant strains of P. aeruginosa (AR Bank #0239) and S. aureus (AR Bank #0565) were obtained from the CDC and FDA Antibiotic Resistant Isolate Bank (Atlanta, GA). Argon (Ar), carbon dioxide (CO₂), nitrogen (N₂), oxygen (O₂), nitric oxide (NO) calibration (25.87 ppm balance N₂), and pure NO (99.5%) gas cylinders were purchased from Airgas National Welders (Raleigh, NC). Distilled water was purified to a resistivity of 18.2 MΩ·cm and a total organic content of ≤6 ppb using a Millipore Milli-Q UV Gradient A10 system (Bedford, MA).

Synthesis of alkylamine-modified glycosaminoglycans. Hyaluronic acid (HA6, HA50, and HA90) and chondroitin sulfate (CSA and CSC) were modified with either HEDA, DPTA, or DETA as adapted from previously published protocols.^{22,68} Briefly, HA or CS (1 g) was dissolved in 40 mL distilled water, or in the case of HA90, 100 mL distilled water, with magnetic stirring. A 4:1 molar ratio of EDC and NHS, with respect to the carboxylic acid moieties on the GAG scaffold, was added. In addition, 1 mL of 0.5 M HCl was added to the HA reactions. Following a 20 min activation period at room temperature, a 4:1 molar ratio of HEDA, DPTA, or DETA, with respect to the carboxylic acid moieties on the GAG scaffold, was added to the reaction solution. The solution was then stirred at room temperature for 48 h. Amine-modified GAGs were precipitated in ethanol, collected via centrifugation, washed twice with ethanol, and dried in vacuo yielding a white/yellow solid for each modification.

Characterization of alkylamine-modified glycosaminoglycans. ¹H and ¹³C nuclear magnetic resonance (NMR) spectra were recorded on a Bruker (600 MHz) spectrometer (Billerica, MA). Attenuated total reflection Fourier transform infrared spectroscopy (ATR-FTIR) was performed using aNicoletiS50 FTIR Spectrometer (Thermo Fisher Scientific; Fair Lawn, NJ). Elemental (carbon, hydrogen, nitrogen, sulfur; CHNS) analysis was conducted using a PerkinElmer Elemental Analyzer Series 2400 Instrument (Waltham, MA). Molecular weight determination was performed by first dissolving the unmodified or amine-modified GAGs at 1 mg mL¹ in 0.1 M phosphate buffer (pH 7.4) containing 0.1 M sodium nitrate and 0.02 wt % sodium azide. Solutions were filtered using a 0.22 μM PTFE filter and analyzed using an aqueous gel permeation chromatography (GPC) system equipped with a Waters 2414 refractive index detector (Milford, MA) coupled to a Wyatt miniDawn TREOS multi-angle light scattering detector (MALS; Santa Barbara, CA) using the same mobile phase as was used for sample preparation.

Synthesis of NO-releasing glycosaminoglycans. Alkylamine-modified HA (45 mg) was dissolved in 7:3 methanol:water (3 mL) with sodium methoxide (NaOMe; 75 μL; 5.4 mM in methanol) in a 1-dram glass vial. Alkylamine-modified CS (45 mg) was dissolved in 1:1 methanol:water (3 mL) with 75 μL NaOMe. The open vials were placed in a stainless-steel reaction vessel and stirred continuously via magnetic stirring. The vessel was purged with argon (10 s, 7 atm) three times followed by three longer argon purges (10 min, 7 atm) to remove excess oxygen in the vessel and in the solutions. The vessel was pressurized to 15 atm with NO gas. After 72 h, the same argon purging procedure was followed to remove unreacted NO. The resulting NO-releasing GAGs were precipitated in ethanol, collected by centrifugation, dried in vacuo, and stored in vacuum sealed bags at −20° C. as a white/yellow powder for each modification.

Characterization of NO-release properties. Absorbance measurements (200-450 nm) were made in 50 mM sodium hydroxide (NaOH) with a Molecular Devices SpectraMax M2 spectrophotometer (San Jose, CA) to confirm the presence of the N-diazeniumdiolate functional group. Real-time nitric oxide release was evaluated using a Sievers 280i Nitric Oxide Analyzer (NOA; Boulder, CO). Before use, samples were analyzed to characterize their NO release and ensure stability of the stored material. The NOA was calibrated with air passed through a NO zero filter (0 ppm NO) and 25.87 ppm of NO calibration gas (balance N₂) prior to analysis. In a typical experiment, NO-releasing GAGs (˜1 mg) were dissolved in either 30 mL of deoxygenated PBS (10 mM, pH 7.4, 37° C.) or 30 mL of deoxygenated simulated wound fluid (SWF; 10% FBS in 10 mM PBS, pH 7.4, 37° C.). For samples analyzed in SWF, 75 μL of antifoam B emulsion was added to the flask. The solution was purged with nitrogen gas at a flow rate of 200 mL min-1 to carry liberated NO to the instrument. Analysis was terminated when NO levels fell below the quantification limit of the instrument normalized to the mass of NO-releasing material (10 ppb NO mg⁻¹ GAG).

In vitro bacterial inhibition assay. Bacteria cultures of PAO1, ATCC MDR-PA, AR-0239, ATCC S. aureus, ATCC MRSA, and AR-0565 were grown from frozen (−80° C.) stocks on TSA plates. Colonies were isolated from the TSA plate and resuspended in SWF (Gram-negative) or SWF supplemented with 10% MHB (Gram-positive) at a concentration of 1×10⁸ CFU mL⁻¹. Bacteria solutions were subsequently diluted to 5×10⁵ CFU mL⁻¹ and exposed to serial dilutions of NO-releasing GAGs (pH adjusted to 7.4 using 1 M HCl), where the highest concentration tested was 16 mg mL⁻¹. The 96-well plate was then incubated at 37° C. for 24 h. Untreated bacteria solutions were included in each experiment to ensure bacteria growth over the 24-h duration. The minimum inhibitory concentration (MIC₂₄ₕ) was defined as the lowest concentration of test agent that prevented visible bacterial growth.

In vitro bacterial eradication assay. Bacteria cultures of PAO1, ATCC MDR-PA, AR-0239, ATCC S. aureus, ATCC MRSA, and AR-0565 were grown from frozen (−80° C.) stocks on TSA plates. Colonies were isolated from the TSA plate, resuspended in TSB (5 mL), and incubated at 37° C. overnight with vigorous (250 rpm) shaking. An aliquot (1 mL) of the overnight bacteria solution was added to fresh TSB (30 mL), grown to an OD₆₀₀ of 0.25 for P. aeruginosa or 1.25 for S. aureus (corresponding to a concentration of 10⁸ CFU mL⁻¹), and subsequently diluted 1:100 to 10⁶ CFU mL⁻¹ in PBS (10 mM, pH 7.4). Weighed samples of NO-releasing GAGs were dissolved in PBS and titrated to pH 7.4 with 1 M HCl. Samples were added to a 96-well polystyrene plate and serially diluted 1:2 in PBS so that each well contained 100 μL of NO-releasing GAG. Bacterial solution containing 10⁶ CFU mL⁻¹ (100 μL; 1 v/v TSB in PBS) was added to each well, resulting in final GAG concentrations of 0.5-16 mg mL⁻¹. Amine-modified (i.e., non-NO-releasing) GAGs were evaluated at 16 mg mL⁻¹ to ensure the GAG backbone was not responsible for bactericidal action. The 96-well plate was then incubated at 37° C. for 4 h with gentle (100 rpm) shaking. Untreated bacteria solutions were included in each experiment to ensure bacteria viability over the 4-h duration. After the 4-h exposure, bacterial solutions were serially diluted (10-, 100-, and 1000-fold dilutions), spiral plated on TSA plates using an Eddy Jet spiral plater (IUL; Farmingdale, NY), and incubated overnight at 37° C. Viability of bacteria following treatment with amine-modified or NO-releasing GAGs was determined using a Flash & Go colony counter (IUL; Farmingdale, NY). The minimum bactericidal concentration after a 4-h exposure period (MBC₄ₕ) was defined as the minimum GAG concentration required to achieve a 3-log reduction (≥99.9% reduction) in bacteria viability relative to untreated bacteria (i.e., reduced bacterial counts from 10⁶ to 10³ CFU mL⁻¹). The limit of detection for this counting method was 2.5×10³ CFU mL⁻¹.^{69,70}

In vitro cytotoxicity assay. Human dermal fibroblasts (HDF) were grown in DMEM supplemented with 10 vol % FBS and 1 wt % PS. Human epidermal keratinocytes (HEK) were grown in keratinocyte SFM with 1 wt % PS and 5 ng mL⁻¹ human recombinant epidermal growth factor (rEGF). Cells were incubated in 5 vol % CO₂ under humidified conditions at 37° C. After reaching 80% confluency, a cell suspension of 1×10⁵ cells mL⁻¹ was prepared, and 100 μL was added to each well of a 96-well plate. After 24-h incubation at 37° C., the supernatant was aspirated and replaced with 100 μL of either unmodified, amine-modified, or NO-releasing GAG in fresh growth medium (pH adjusted to 7.4 with 1 M HCl) with GAG concentrations ranging from 0.25 to 32 mg mL⁻¹. The cultures were then incubated for 24 h at 37° C. Following exposure, the supernatant was aspirated, and the wells were washed twice with PBS. A 100-μL mixture of growth medium/MTS/PMS (105/20/1, v/v/v) was added to each well and incubated for 90 min at 37° C. The absorbance of the solution in each well was measured at 490 nm using a Molecular Devices SpectraMax M2 spectrophotometer (San Jose, CA). A blank mixture of growth medium/MTS/PMS and untreated cells were used as the blank and control, respectively. Cell viability for each sample was calculated using Eq. 1.

$$\% cell viability \frac{( {Abs}_{490 , sample} - Abs \text{?} )}{( Abs \text{?} - Abs \text{?} )} \times 100 \qquad ( Eq . 1 )$$

$$\text{?} \text{indicates text missing or illegible when filed}$$

In vitro inflammation assay. HEK-BLUE mTLR4 cells were grown in DMEM supplemented with 10 vol % FBS, 1 wt % PS, 100 μg mL⁻¹ NORMOCIN, and 1×HEK-BLUE Selection. Cells were incubated in 5 vol % CO₂ under humidified conditions at 37° C. After reaching 80% confluency, a cell suspension of 1.4×10⁵ cells mL⁻¹ was prepared. Fresh media (20 μL), either blank or containing test agent at 10× concentration, was added to the wells of a 96-well plate. The cell suspension (180 μL) was added to each well, with a final concentration of cells in each well of 2.5×10⁴ cells well⁻¹. Unmodified, amine-modified, and NO-releasing GAG samples were evaluated at final well concentrations of 100 ng mL⁻¹ to 100 μg mL⁻¹. Positive controls included 100 μg mL⁻¹ to 100 ng mL⁻¹ of LPS and TNF-α to ensure NF-κB activation and to validate the assay. After incubation for 24 h at 37° C., 20 μL of the supernatant from each well was removed and added to 180 μL QUANTI-BLUE solution, prepared according to the manufacturer instructions. Plates were incubated for 1 h, and the absorbance of the solution in each well was measured at 630 nm using a Molecular Devices SpectraMax M2 spectrophotometer (San Jose, CA). Blank media and untreated cells were used as the blank and negative control, respectively. The relative concentration of secreted embryonic alkaline phosphatase (SEAP), produced as a result of NF-κB activation, is presented as the OD₆₃₀ corrected for the blank.

BEK-BLUE Null1-v cells that express endogenous levels of TLR3, TLR5, NOD1, ALPK1, and TIFA but are not transfected with the murine TLR4 receptor gene were used as a control to ensure measured activity was due to interaction with the TLR4 receptor. BEK-BLUE Null1-v cells were grown in DMEM supplemented with 10 vol % FBS, 1 wt % PS, and 100 μg mL⁻¹ ZEOCIN and utilized in the same manner for NF-κB activation experiments as described above.

In vitro adhesion assay. Adhesion assays were carried out using a procedure adapted from Kucik and Wu.⁷¹ Briefly, 96-well plates were coated with 100 μL of 32 μg mL⁻¹ bovine collagen type I (10 μg cm⁻¹ growth area) in PBS and incubated for 1 h at 37° C. After 1 h, collagen solution was aspirated from the wells and replaced with 100 μL of 10 mg mL⁻¹ BSA in PBS for 1 h at 37° C. In tandem, 1.5-mL microcentrifuge tubes were coated with BSA using the same procedure at a greater volume (1.5 mL BSA solution). Following incubation, each plate and microcentrifuge tube was rinsed once with PBS and once with the respective growth medium. Fresh growth medium, either blank or containing unmodified, amine-modified, or NO-releasing GAG, was added to the well plate (100 μL). HDF and HEK were grown under described conditions until 80% confluent. A cell suspension of 1×10⁵ or 1.5×10⁵ cells mL⁻¹ was prepared for HDF and HEK, respectively. An aliquot of the cell suspension (100 μL) was added to each well, with final GAG concentrations spanning the range of 100 ng mL⁻¹ to 100 μg mL⁻¹, and the plate was incubated for 1 h at 37° C. Simultaneously, a 1-mL aliquot of the cell suspension was transferred to a BSA-coated 1.5-mL microcentrifuge tube. Cells were immediately pelleted at 3800×g for 15 min, the supernatant was removed, and the pellet was stored at −20° C. until needed. After 1 h, the 96-well plate was rinsed thrice with PBS to remove non-adhered cells, and a 100 μL mixture of growth medium/MTS/PMS (105/20/1, v/v/v) was added to each well. Growth medium/MTS/PMS solution was added to the empty wells as a blank. Growth medium/MTS/PMS (1 mL) was added to the cell pellet, which was gently vortexed and added to the well plate (100 μL well-1) as a measure of the total number of cells initially added to each well. The plate was incubated for 90 min at 37° C., and the absorbance of the solution in each well was measured at 490 nm using a Molecular Devices SpectraMax M2 spectrophotometer (San Jose, CA). Cell adhesion for each sample was calculated using Eq. 2.

$$\% cell adhesion = \frac{( {Abs}_{490 , adhered} - {Abs}_{490 , blank} )}{( {Abs}_{490 , total} - {Abs}_{490 , blank} )} \times 100 \qquad ( Eq . 2 )$$

In vitro proliferation assay. HDF and HEK were grown under described conditions until 80% confluent. A cell suspension of 3×10⁴ or 7.5×10⁴ cells mL⁻¹ was prepared for HDF and HEK, respectively. An aliquot of the cell suspension (100 μL) was added to each well of a 96-well plate, and plates were incubated at 37° C. for 24 h to ensure adhesion. After 24 h, the supernatant was aspirated and replaced with 200 μL of fresh growth medium, either blank or containing unmodified, amine-modified, or NO-releasing GAGs spanning the range of 100 ng mL⁻¹ to 100 μg mL⁻¹. Importantly, after the initial 24 h of cell adhesion, growth medium was prepared without either FBS (for HDF) or rEGF (for HEK) to prevent excess unstimulated proliferation. The plate was then incubated for 72 h at 37° C. Following exposure, the supernatant was aspirated, and the wells were washed twice with PBS. A 100 μL mixture of growth medium/MTS/PMS (105/20/1, v/v/v) was added to each well and incubated for 90 min at 37° C. The absorbance of the solution in each well was measured at 490 nm using a Molecular Devices SpectraMax M2 spectrophotometer (San Jose, CA). A mixture of growth medium/MTS/PMS without cells and untreated cells were used as the blank and control, respectively. Cell proliferation, relative to untreated cells, was calculated using E q. 3.

$$\% relative proliferation = \frac{( {Abs}_{490 , sample} - {Abs}_{490 , blank} )}{( {Abs}_{490 , control} - {Abs}_{490 , blank} )} \times 100 \qquad ( Eq . 3 )$$

In vivo murine wound healing model. The animal studies were approved and carried out in compliance with the Institutional Animal Care and Use Committee standards (IACUC approval number 21-082). Female C57B6/Ntac wild-type mice (body weight of ˜20 g; 8-weeks old) were purchased from Taconic Farms (Rensselaer, NY). Mice were housed individually with 12-h light-dark cycles. For wounding, mice were anesthetized using gaseous isoflurane, hair was removed from the dorsal region of the mouse using clippers and a depilatory cream, and the skin was prepared for surgery using povidone-iodine and 70 vol % ethanol. A sterile 6-mm biopsy punch was used to outline a circular pattern between the shoulders. Forceps were used to lift the skin, and surgical scissors were used to create a full-thickness wound on each mouse. Next, P. aeruginosa (PAO1, 25 μL of 8×10⁶ CFU mL⁻¹) was pipetted into the wound to facilitate infection. For the first study, mice were randomly assigned into 3 treatment groups of 5 mice each and treated with 10 μL of either PEG 400, 100 mg mL⁻¹ HA6-HEDA/NO in PEG 400, or 100 mg mL⁻¹ CSC-HEDA/NO in PEG 400. For the subsequent study, mice were randomly assigned into 5 treatment groups of 5 mice each and treated with 10 μL of either PEG 400, 100 mg mL⁻¹ CSC-HEDA in PEG 400, 100 mg mL⁻¹ CSC-HEDA/NO in PEG 400, 100 mg mL⁻¹ CSC-DPTA in PEG 400, or 100 mg mL⁻¹ CSC-DPTA/NO in PEG 400. Treatment began immediately following wounding and continued once daily for 7 d post-wounding. Throughout the experiment, mice were monitored and received acetaminophen in their drinking water (1.6 mg mL⁻¹) ad libitum. Each day, all wounds were photographed and measured in perpendicular directions using calipers for wound area calculations.

On day 8 post-wounding, mice were sacrificed via carbon dioxide inhalation/cervical dislocation and residual wound tissue was harvested, stored at −80° C., and processed for DNA extraction using a DNeasy Blood and Tissue Kit. To quantify levels of P. aeruginosa (strain PAO1) via quantitative PCR (qPCR), two primer sets were utilized to anneal and amplify the P. aeruginosa oprL gene: PAO1 S^{a} (5′ ACCCGAACGCAGGCTATG 3′), PAO1 Aa (5′ CAGGTCGGAGCTGTCGTACTC 3′), oprL Fa (5′ ATGGAAATGCTGAAATTCGGC 3′), oprL R^{a} (5′ CTTCTTCAGCTCGACGCGACG 3′). Each reaction was performed in triplicate, contained 1 μg purified DNA (6.5 μL), 12.5 μL iQ SYBR Green Supermix, 0.5 μL forward primer (100 μM), and 0.5 μL reverse primer (100 μM), and was analyzed using a CFX96 Real-Time PCR Detection System (Bio-Rad Laboratories, Inc.; Hercules, CA). The average cycle threshold (CT) value for each reaction was subtracted from 40. Technical triplicates were averaged to obtain relative expression of PAO1 16s rRNA within the wound of each animal.

Data analysis. NO-release and elemental analysis measurements are presented herein as the average±standard deviation from n≥3 separate synthesis batches. Bacteria viability, cytotoxicity, cell proliferation, and cell adhesion results are depicted herein as the average±standard deviation from n≥3 separate experiments with bacteria/mammalian cells grown on different days. Dose-response curves for mammalian cell viability were plotted using GraphPad Prism 9 software (San Diego, CA). Nonlinear regression (normalized response with variable slope) analysis was performed to determine IC₅₀ values. In vivo wound-healing data are presented herein as the average±standard deviation from n=5 separate animals. Significance testing for cell adhesion, proliferation, inflammation, and migration assays was performed via a 2-tailed Student's t-test. Significance levels are denoted: *p<0.05. Statistical analysis of wound closure and residual PAO1 genome for the in vivo study was performed using a one-way ANOVA with Tukey's multiple comparisons. Significance levels are denoted: *p<0.05, **p<0.01, ***p<0.005.

### Results and Discussion

The modification of HA (6, 50, and 90 kDa) and CS (30 kDa CSA and 20 kDa CSC) with alkylamine groups for subsequent N-diazeniumdiolate NO donor formation was evaluated. These HA molecular weights were selected for study based on their potential to interact with one or more of the main receptors for HA's wound healing pathways (TLR, RHAMM, and CD44 receptors).^{47,50,51,72} The two CS isomers, CSA and CSC, were chosen as they represent the most abundant forms of CS (from animal tissue) and may differ in wound-healing properties as a result of their sulfation pattern.^{49,58,59} The alkylamine groups used for modification were described previously to confer a range of NO-release kinetics.^{22,68,73} Glycosaminoglycan derivatives varying in biopolymer physical properties (i.e., GAG identity, HA molecular weight, CS sulfation pattern, alkylamine identity) and NO-release properties (i.e., NO payloads and kinetics) were screened for antibacterial and pro-wound healing properties using several in vitro assays. Antibacterial properties, including bacterial inhibition and eradication, were tested against prevalent wound pathogens, including clinical multidrug-resistant P. aeruginosa and S. aureus isolates. The roles of biopolymer physical properties and NO-release properties on inflammation were investigated using an in vitro assay via activation of the murine TLR4 receptor. Aspects of wound healing, including cell proliferation and adhesion, were evaluated in vitro with human dermal fibroblasts and human epidermal keratinocytes as representative cell types in the wound environment. The in vitro antibacterial, wound healing, and inflammation properties of the most promising candidates were evaluated in a P. aeruginosa-infected murine wound model.

### Example 1: Modification of Glycosaminoglycans with Alkylamines

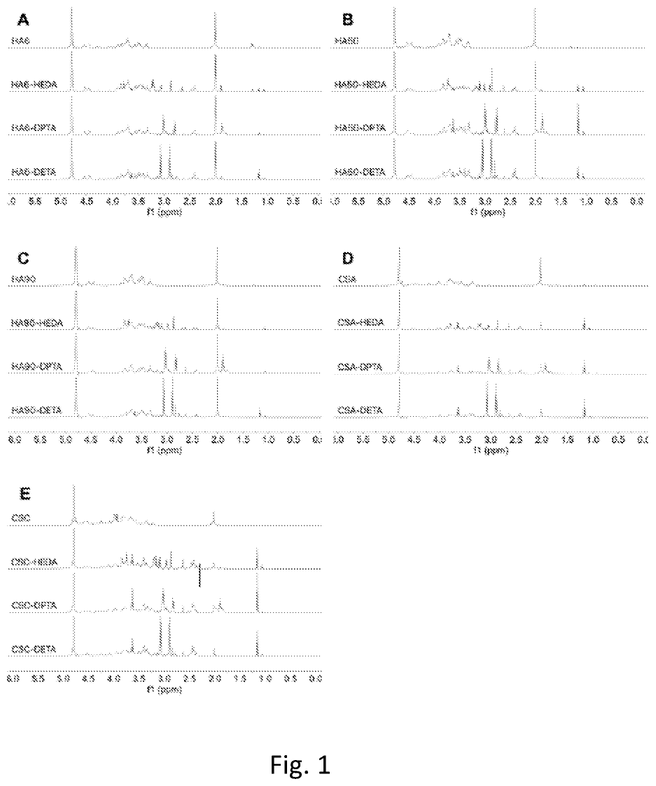

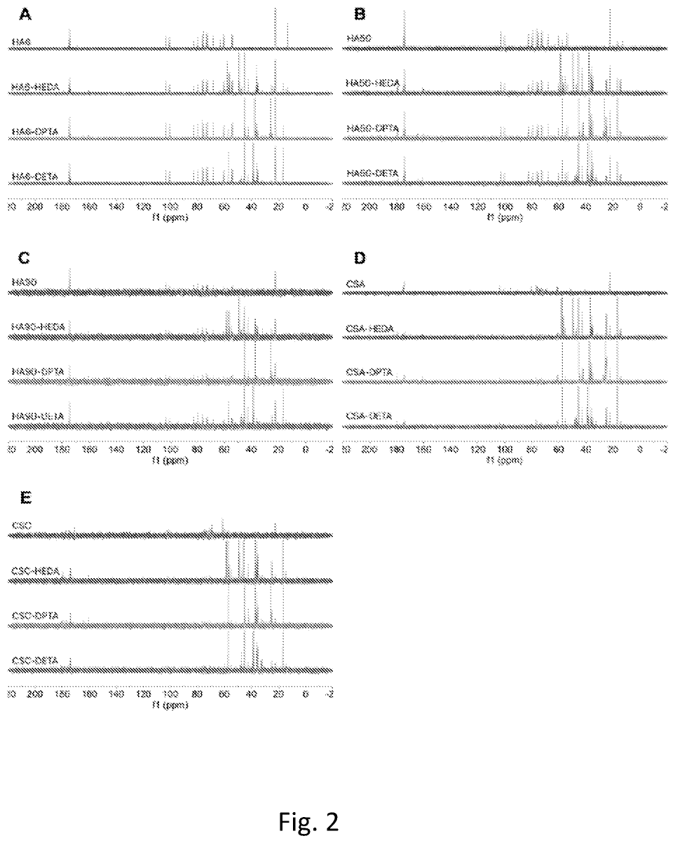

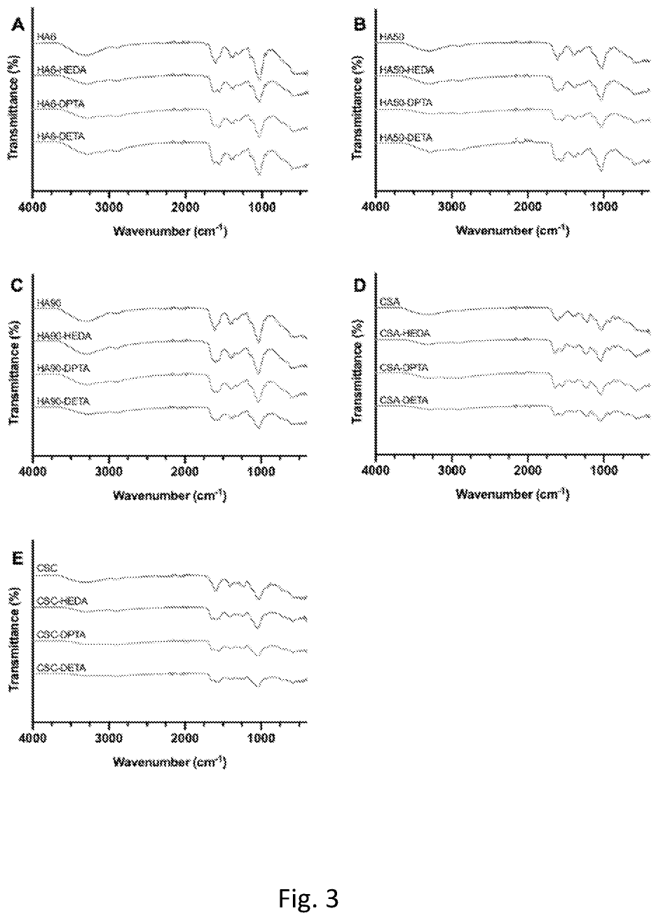

Carbodiimide chemistry was used to graft a series of alkylamines, including N-(2-hydroxyethyl)ethylenediamine (HEDA), bis(3-aminopropyl)amine (DPTA), and diethylenetriamine (DETA), onto five GAG backbones (Scheme 1). Three hyaluronic acid derivatives, HA6 (12.2 kDa, D=1.52), HA50 (52.0 kDa, D=2.47), and HA90 (95.1 kDa, D=1.39), and two chondroitin sulfate derivatives, CSA (29.2 kDa, D=1.53) and CSC (17.0 kDa, D=2.27), were included to examine the role of molecular weight and sulfation pattern, respectively, on antibacterial and wound healing properties (Table 1). Successful incorporation of the alkylamine functional groups was monitored using ¹H and ¹³C NMR spectroscopy (FIGS. 1 and 2), FTIR spectroscopy (FIG. 3), and CHNS elemental analysis (Table 2). The addition of a peak at 160 ppm in the ¹³C NMR spectra indicates the formation of amide bonds, confirming success of the carbodiimide reaction (FIG. 2). Further, the peak in the carbonyl region of the FTIR spectra (1550-1700 cm⁻¹) demonstrates a marked change in shape, supporting an increase in amide content (˜1580 cm⁻¹) (FIG. 3). Elemental analysis (CHNS) was used to assess the extent to which the GAGs were modified with alkylamines. Glycosaminoglycan derivatives increased from 2-4 wt % to 7-18 wt % nitrogen, with CSC derivatives showing the greatest increase in nitrogen content (Table 2). The range of molecular weights was retained for all derivatives following modification with alkylamines, with each amine-modified derivative possessing similar to or greater than the molecular weight of the unmodified GAG (Table 1).

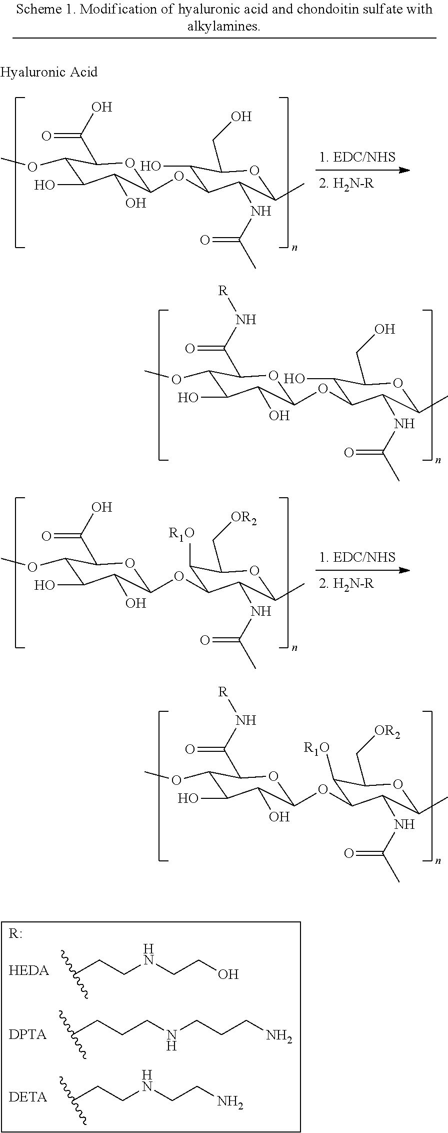

TABLE 1
Representative weight-average molecular weights (Mw)
and dispersity (Ð) of unmodified and amine-modified
hyaluronic acid and chondroitin sulfate.^{a}

| Modification | M_{w} (kDa) | Ð |
|---|---|---|
| HA6 | 12.2 | 1.52 |
| HA50 | 52.0 | 2.47 |
| HA90 | 95.1 | 1.39 |
| CSA | 29.2 | 1.53 |
| CSC | 17.0 | 1.97 |
| HA6-HEDA | 10.7 | 1.39 |
| HA50-HEDA | 59.3 | 2.55 |
| HA90-HEDA | 122.8 | 2.36 |
| CSA-HEDA | 45.7 | 1.52 |
| CSC-HEDA | 22.1 | 2.27 |
| HA6-DPTA | 13.9 | 1.18 |
| HA50-DPTA | 58.9 | 1.57 |
| HA90-DPTA | 95.8 | 1.62 |
| CSA-DPTA | 37.3 | 1.42 |
| CSC-DPTA | 37.5 | 1.35 |
| HA6-DETA | 11.6 | 1.47 |
| HA50-DETA | 47.3 | 2.16 |
| HA90-DETA | 89.7 | 1.90 |
| CSA-DETA | 34.7 | 1.71 |
| CSC-DETA | 50.8 | 1.21 |

^{a}Determined by GPC-MALS in 0.1M phosphate buffer (pH 7.4) supplemented with 0.1M NaNO3 and 0.02 wt % NaN₃.

TABLE 2
Elemental analysis (CHNS) of unmodified and amine-
modified hyaluronic acid and chondroitin sulfate.^{a}

| Modification | C (%) | H (%) | N (%) | S (%) |
|---|---|---|---|---|
| HA6 | 39.2 ± 0.8 | 6.2 ± 0.3 | 3.1 ± 0.1 | N/A |
| HA50 | 37.2 ± 0.1 | 5.5 ± 0.2 | 3.0 ± 0.1 | N/A |
| HA90 | 35.5 ± 1.7 | 5.9 ± 0.2 | 2.9 ± 0.1 | N/A |
| CSA | 29.0 ± 0.1 | 4.6 ± 0.1 | 4.0 ± 0.1 | 4.3 ± 0.5 |
| CSC | 32.9 ± 0.1 | 5.2 ± 0.1 | 1.7 ± 0.1 | 1.3 ± 0.4 |
| HA6-HEDA | 40.8 ± 0.9 | 6.9 ± 0.2 | 7.8 ± 0.1 | N/A |
| HA50-HEDA | 41.4 ± 0.5 | 6.6 ± 0.1 | 7.9 ± 0.3 | N/A |
| HA90-HEDA | 40.3 ± 0.6 | 6.7 ± 0.3 | 7.1 ± 0.1 | N/A |
| CSA-HEDA | 35.6 ± 0.4 | 5.7 ± 0.4 | 9.5 ± 0.1 | 3.5 ± 0.1 |
| CSC-HEDA | 39.3 ± 0.1 | 5.9 ± 0.1 | 12.8 ± 0.9 | 1.4 ± 0.1 |
| HA6-DPTA | 41.6 ± 0.8 | 7.4 ± 0.1 | 8.7 ± 0.1 | N/A |
| HA50-DPTA | 42.7 ± 1.3 | 7.6 ± 0.1 | 9.2 ± 0.1 | N/A |
| HA90-DPTA | 40.8 ± 0.3 | 6.8 ± 0.5 | 8.1 ± 0.2 | N/A |
| CSA-DPTA | 37.3 ± 0.3 | 6.6 ± 0.2 | 11.1 ± 0.9 | 3.4 ± 0.3 |
| CSC-DPTA | 40.8 ± 0.2 | 6.5 ± 0.2 | 16.3 ± 1.8 | 1.4 ± 0.2 |
| HA6-DETA | 41.4 ± 0.5 | 7.4 ± 0.2 | 10.2 ± 0.2 | N/A |
| HA50-DETA | 41.6 ± 0.7 | 7.0 ± 0.3 | 10.2 ± 0.3 | N/A |
| HA90-DETA | 40.5 ± 0.6 | 7.5 ± 0.2 | 9.1 ± 0.2 | N/A |
| CSA-DETA | 35.9 ± 0.9 | 6.2 ± 0.1 | 13.7 ± 2.7 | 3.3 ± 0.5 |
| CSC-DETA | 39.6 ± 0.3 | 6.5 ± 0.1 | 17.7 ± 2.2 | 1.3 ± 0.1 |

^{a}Error represents standard deviation for n ≥ 3 separate syntheses.

### Example 2: NO-Release Properties of Glycosaminoglycans

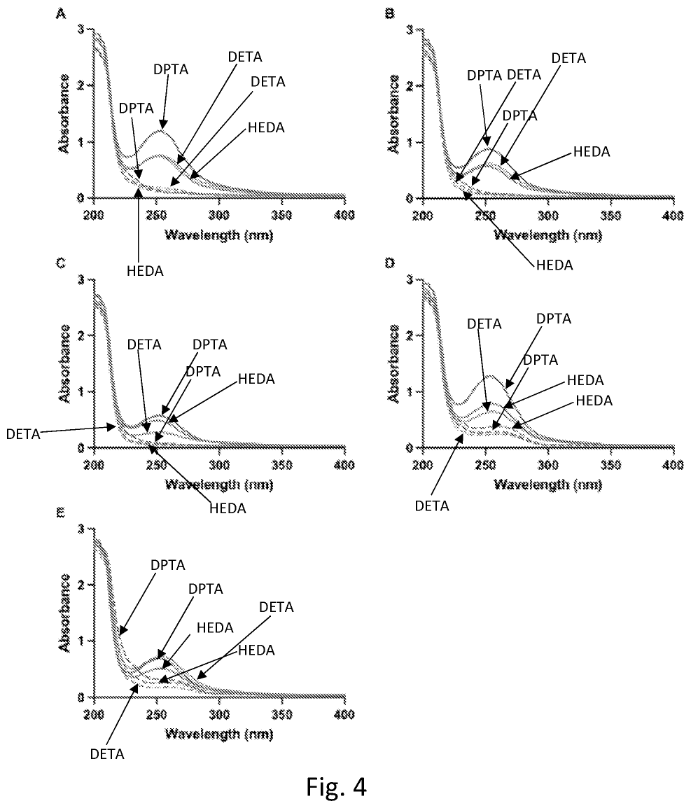

The formation of N-diazeniumdiolate NO donors was carried out via exposure to high pressures of NO gas under basic conditions. N-diazeniumdiolate NO donor formation was confirmed using UV-Vis spectroscopy, with the appearance of a characteristic absorbance peak at 250-255 nm observed for each of the NO-releasing GAG derivatives (FIG. 4). Of note, no peaks were observed for amine-modified (control) HA scaffolds in this region; however, amine-modified CS derivatives displayed a slight peak at 258-260 nm as a result of the sulfate groups on the backbone. The release of NO from the GAG scaffolds was directly monitored in real-time using a chemiluminescent nitric oxide analyzer (NOA), with each derivative evaluated in both PBS and simulated wound fluid (SWF; 10 vol % FBS in PBS). Nitric oxide payloads ranged from 0.2 to 0.8 μmol NO mg⁻¹ GAG in PBS (pH 7.4) depending on the alkylamine modification, HA molecular weight, and CS sulfation pattern (Table 3). Glycosaminoglycans modified with DETA exhibit lower NO release payloads than HEDA- and DPTA-modified GAGs, which can be attributed to the stability of the NO donor. It is likely that more NO is released by these DETA-modified biopolymers at levels unmeasurable by the NOA after analysis is terminated. For DPTA- and DETA-modified HA, while not statistically significant, a slight decrease in NO payload was observed with increasing molecular weight. A significant (p<0.05) decrease in NO payload was found for HA50-HEDA/NO as compared to HA6-HEDA/NO. Greater chain entanglement and viscosity with increasing HA molecular weight may decrease accessibility of NO to the precursor NO donor moiety during the N-diazeniumdiolate formation process. Each of the CSC derivatives studied exhibited greater alkylamine functionalization (Table 4), and therefore greater NO payloads (Table 3), than all the CSA derivatives. This discrepancy between the CS isomers is likely a result of sulfation pattern.

TABLE 3
Nitric oxide-release properties of GAG derivatives in
PBS (10 mM, pH 7.4, 37° C.) and simulated wound fluid
(10% v/v FBS in PBS, 37° C.).^{a}

| | Phosphate Buffered Saline | | Simulated Wound Fluid | |
|---|---|---|---|---|
| | [NO]ₜ | | [NO]ₜ | |
| | (μmol | t_{1/2} | (μmol | t_{1/2} |
| Modification | mg⁻¹)^{b} | (min)^{c} | mg⁻¹)^{b} | (min)^{c} |
| HA6-HEDA/NO | 0.45 ± 0.01 | 19 ± 2 | 0.40 ± 0.04 | 39 ± 17 |
| HA50-HEDA/NO | 0.34 ± 0.03 | 19 ± 1 | 0.34 ± 0.05 | 26 ± 6 |
| HA90-HEDA/NO | 0.42 ± 0.07 | 22 ± 3 | 0.39 ± 0.04 | 53 ± 13 |
| CSA-HEDA/NO | 0.31 ± 0.01 | 16 ± 1 | 0.22 ± 0.02 | 24 ± 3 |
| CSC-HEDA/NO | 0.46 ± 0.15 | 14 ± 1 | 0.38 ± 0.11 | 18 ± 4 |
| HA6-DPTA/NO | 0.51 ± 0.10 | 25 ± 2 | 0.51 ± 0.10 | 46 ± 11 |
| HA50-DPTA/NO | 0.46 ± 0.05 | 29 ± 6 | 0.43 ± 0.05 | 37 ± 6 |
| HA90-DPTA/NO | 0.35 ± 0.08 | 34 ± 5 | 0.34 ± 0.03 | 62 ± 16 |
| CSA-DPTA/NO | 0.48 ± 0.08 | 19 ± 1 | 0.42 ± 0.01 | 24 ± 6 |
| CSC-DPTA/NO | 0.78 ± 0.08 | 16 ± 3 | 0.91 ± 0.04 | 20 ± 4 |
| HA6-DETA/NO | 0.31 ± 0.01 | 60 ± 2 | 0.31 ± 0.05 | 84 ± 2 |
| HA50-DETA/NO | 0.28 ± 0.03 | 66 ± 20 | 0.27 ± 0.06 | 89 ± 3 |
| HA90-DETA/NO | 0.27 ± 0.03 | 75 ± 13 | 0.24 ± 0.02 | 76 ± 13 |
| CSA-DETA/NO | 0.26 ± 0.03 | 51 ± 6 | 0.22 ± 0.04 | 67 ± 13 |
| CSC-DETA/NO | 0.30 ± 0.07 | 88 ± 7 | 0.31 ± 0.11 | 108 ± 8 |

^{a}Error represents the standard deviation of n ≥ 3 separate syntheses.
^{b}Total NO released over release duration.
^{c}Half-life of NO release.

TABLE 4
Nitric oxide-release properties of GAG derivatives in PBS (10 mM, pH 7.4, 37°
C.) simulated wound fluid (10% v/v FBS in PBS, 37° C.).^{a}

| | Phosphate Buffered Saline | | Simulated Wound Fluid | |
|---|---|---|---|---|
| | [NO]ₘₐₓ | | [NO]ₘₐₓ | |
| | (ppb | t_{d} | (ppb | t_{d} |
| Modification | mg⁻¹)^{b} | (h)^{c} | mg⁻¹)^{b} | (h)^{c} |
| HA6-HEDA/NO | 2270 ± 390 | 5.7 ± 1.4 | 1200 ± 360 | 7.6 ± 1.2 |
| HA50-HEDA/NO | 1460 ± 90 | 4.7 ± 0.3 | 940 ± 280 | 4.3 ± 0.4 |
| HA90-HEDA/NO | 1440 ± 310 | 5.5 ± 0.8 | 660 ± 170 | 7.5 ± 1.9 |
| CSA-HEDA/NO | 1600 ± 130 | 4.6 ± 0.3 | 610 ± 50 | 3.0 ± 0.5 |
| CSC-HEDA/NO | 2400 ± 980 | 3.5 ± 0.4 | 1600 ± 610 | 2.9 ± 0.8 |
| HA6-DPTA/NO | 2440 ± 580 | 8.1 ± 0.7 | 1290 ± 100 | 12.0 ± 1.6 |
| HA50-DPTA/NO | 1640 ± 480 | 7.2 ± 0.5 | 1120 ± 100 | 8.3 ± 1.3 |
| HA90-DPTA/NO | 1090 ± 240 | 6.9 ± 1.4 | 620 ± 40 | 10.7 ± 3.3 |
| CSA-DPTA/NO | 2560 ± 590 | 6.5 ± 0.6 | 1520 ± 310 | 5.4 ± 0.1 |
| CSC-DPTA/NO | 4450 ± 890 | 5.2 ± 0.6 | 3740 ± 590 | 6.0 ± 0.3 |
| HA6-DETA/NO | 1490 ± 340 | 12.2 ± 0.4 | 910 ± 190 | 13.3 ± 1.8 |
| HA50-DETA/NO | 1300 ± 330 | 11.6 ± 0.6 | 680 ± 210 | 12.2 ± 1.2 |
| HA90-DETA/NO | 1000 ± 360 | 12.3 ± 1.2 | 670 ± 30 | 11.5 ± 1.6 |
| CSA-DETA/NO | 1090 ± 270 | 9.8 ± 0.7 | 600 ± 100 | 9.5 ± 1.3 |
| CSC-DETA/NO | 670 ± 350 | 11.6 ± 0.9 | 510 ± 240 | 12.8 ± 2.6 |

^{a}Error represents the standard deviation of n ≥ 3 separate syntheses.
^{b}Maximum instantaneous NO concentration.
^{c}Duration of NO release.

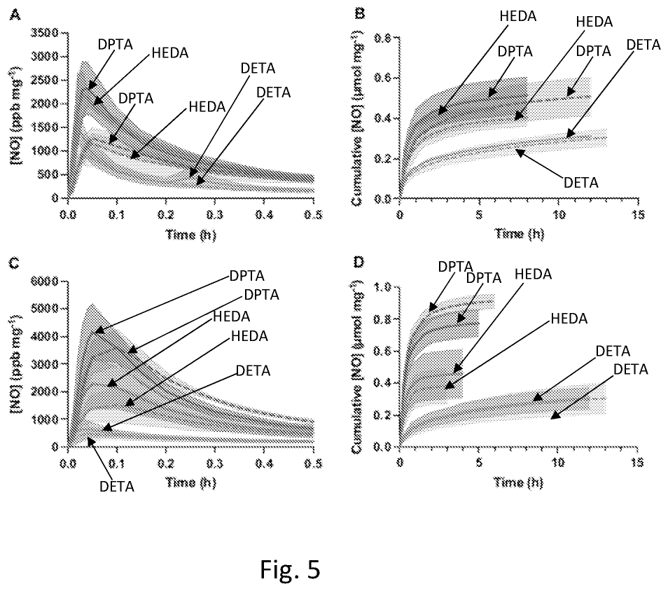

The incorporation of three alkylamine substituents to the GAG backbones confers a range of NO-release kinetics in PBS (FIG. 5). Nitric oxide-releasing GAGs modified with HEDA exhibited the shortest half-lives and durations in PBS (Tables 3 and 4, FIG. 5), owing to the reduced stabilization of the N-diazeniumdiolate moiety by the terminal hydroxyl group. Increased NO donor stabilization, and thus extended NO-release durations, were observed with the primary amine-terminated derivatives, DPTA and DETA. However, DETA resulted in even longer NO release as compared to DPTA (e.g., 8.1±0.7 vs. 12.2±0.4 h durations for HA6-DPTA/NO and HA6-DETA/NO, respectively), suggesting that the ethyl spacing between the NO donor-containing secondary amine and positively charged tertiary amine of DETA facilitates better stabilization than the propyl spacing of DPTA.

The kinetics of NO release changed when analyzed in SWF versus PBS. For all derivatives, the maximum instantaneous concentration of NO release was less in SWF compared to PBS (Table 4, FIG. 5). As a result, the half-lives of NO release are extended for the majority of the GAG derivatives (Table 3). The components of FBS (e.g., glucose, proteins, enzymes, counterions) within SWF may affect stabilization of the N-diazeniumdiolate NO donor, altering the kinetics of NO release. However, the majority of NO payloads were retained when evaluated in SWF as compared to PBS, indicating negligible scavenging from the protein components in FBS (Table 3). As a result, NO totals should not be substantially altered when used in a wound environment even if there are 10-60% changes to the maximum flux and duration of NO release.

### Example 3: In Vitro Inhibitory and Eradication Activity Against Common Wound Pathogens

In developing a new chronic wound therapy, it is useful to consider the antibacterial activity of the proposed material. Multiple isolates of P. aeruginosa and S. aureus were thus evaluated for both inhibition and eradication following treatment with NO-releasing GAGs. Three strains of each species were chosen to encompass a range of antibiotic-resistance profiles, including two susceptible strains (PAO1 and ATCC S. aureus), two ATCC resistant strains (ATCC MDR-PA and ATCC MRSA), and two multidrug-resistant clinical isolates provided by the Centers for Disease Control (AR-0239 and AR-0565). Of note, P. aeruginosa strain AR-0239 and S. aureus strain AR-0565 are reported to be resistant to ≥15 and >10 common antibiotics, respectively. The use of these multidrug-resistant strains facilitates an evaluation of the NO-releasing GAGs on several acquired resistance mechanisms.

The inhibitory action of the NO-releasing GAG derivatives was first evaluated against the six wound pathogens. Bacteria were treated with NO-releasing GAGs in simulated wound fluid to determine the minimum inhibitory concentration, or MIC₂₄ₕ, defined as the concentration of GAG required to prevent visible bacterial growth over 24 h. In evaluating the Gram-negative P. aeruginosa strains (i.e., PAO1, ATCC MDR-PA, and AR-0239), DETA-modified NO-releasing GAGs proved the most effective at preventing bacterial growth for each strain compared to the HEDA- and DPTA-modified derivatives (Table 5). The efficacy of this inhibition trended with NO-release duration. Derivatives with the shortest NO-releasing lifetime, HEDA-modified GAGs, required the largest concentrations of GAGs to inhibit P. aeruginosa growth. Derivatives with intermediate NO-release durations and half-lives (i.e., DPTA-modified GAGs) required intermediate GAG concentrations to inhibit bacterial growth (4-16 mg mL⁻¹). The sustained release of DETA-modified derivatives (9-13 h in SWF) was more effective in preventing bacterial growth over a 24-h experiment duration. Increased NO-releasing DETA-modified GAG concentrations was necessary to prevent bacterial growth in ATCC MDR-PA and AR-0239 in comparison to PAO1, suggesting that the resistance mechanisms adopted by the bacteria, such as the production of mucoid exopolysaccharide capsule,⁷⁴ may facilitate decreased permeability of NO through the bacterial cell membrane. However, these same strain differences were not observed for HEDA- and DPTA-modified GAGs. Of note, NO-releasing DETA-modified GAGs were still able to inhibit all P. aeruginosa strains at concentrations ≤8 mg mL⁻¹. In contrast to the Gram-negative pathogens, Gram-positive S. aureus strains (i.e., ATCC S. aureus, ATCC MRSA, and AR-0565) were equally inhibited by all GAG derivatives, with each strain requiring 1-4 mg mL⁻¹ of NO-releasing GAG to inhibit bacterial growth over 24 h (Table 5), indicating that even short-term NO release (3-8 h with HEDA-modified derivatives) is sufficient to inhibit the growth of S. aureus derivatives during the experimental timeframe. Negligible differences in MIC₂₄ₕ were observed across the three S. aureus strains, which varied in resistance profile, indicating that the resistance mechanisms adopted by ATCC MRSA and AR-0565 do not affect the ability of NO to inhibit growth.

TABLE 5
Minimum inhibitory concentrations (MIC_{24 h}) of NO-releasing
glycosaminoglycan derivatives against antibiotic
susceptible and drug-resistant strains of common wound pathogens
in simulated wound fluid (10% v/v SWF in PBS)^{a} following
24-h exposure.^{b}

| | MIC_{24 h} (mg mL⁻¹) | | | | | |
|---|---|---|---|---|---|---|
| | | | | ATCC | | |
| | | ATCC | AR- | S. | ATCC | AR- |
| Modification | PAO1 | MDR-PA | 0239 | aureus | MRSA | 0565 |
| HA6-HEDA/NO | 8 | 16 | 16 | 4 | 2 | 2 |
| HA50-HEDA/NO | 16 | 16 | 16 | 4 | 4 | 4 |
| HA90-HEDA/NO | >16 | 16 | 4 | 2 | 2 | 2 |
| CSA-HEDA/NO | 16 | >16 | 16 | 4 | 4 | 4 |
| CSC-HEDA/NO | 8 | 16 | 16 | 4 | 4 | 4 |
| HA6-DPTA/NO | 8 | 8 | 8 | 2 | 2 | 1 |
| HA50-DPTA/NO | 8 | 8 | 4 | 2 | 2 | 1 |
| HA90-DPTA/NO | 8 | 16 | 4 | 2 | 1 | 2 |
| CSA-DPTA/NO | 8 | 16 | 8 | 2 | 4 | 2 |
| CSC-DPTA/NO | 8 | 8 | 8 | 2 | 2 | 2 |
| HA6-DETA/NO | 1 | 4 | 4 | 2 | 2 | 2 |
| HA50-DETA/NO | 1 | 4 | 4 | 4 | 2 | 1 |
| HA90-DETA/NO | 2 | 8 | 2 | 2 | 1 | 2 |
| CSA-DETA/NO | 2 | 4 | 4 | 4 | 4 | 4 |
| CSC-DETA/NO | 1 | 4 | 8 | 2 | 2 | 1 |

^{a}SWF was supplemented with 10% v/v Mueller-Hinton II broth for S. aureus strains.
^{b}MIC_{24 h} values were determined from n ≥ 3 separate experiments.

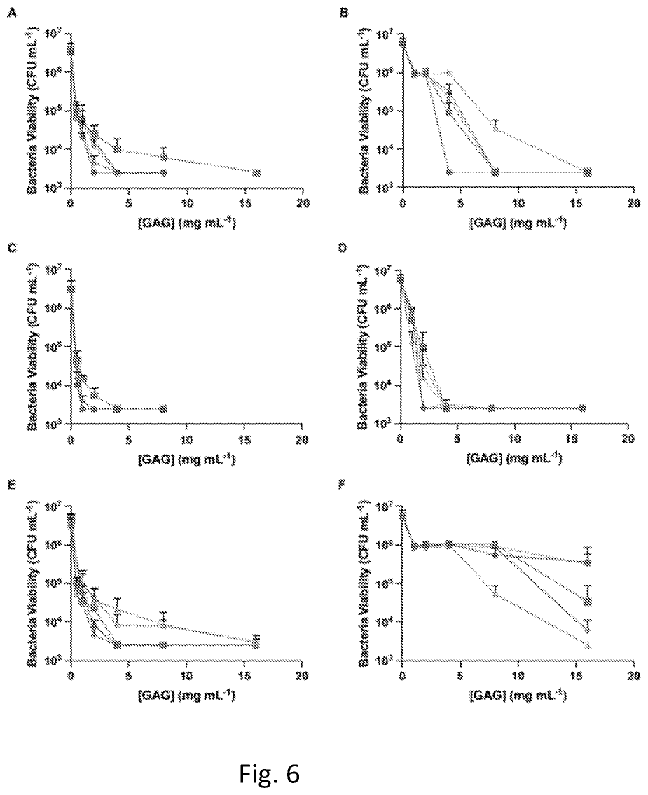

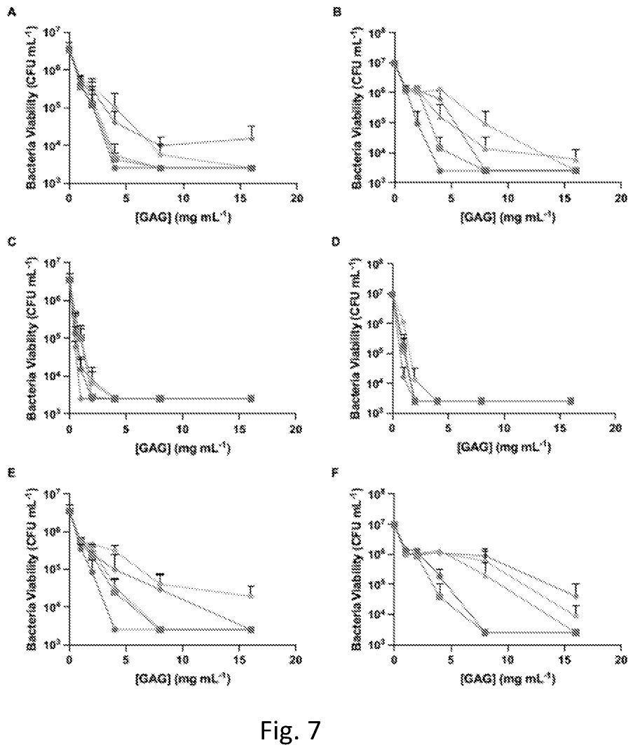

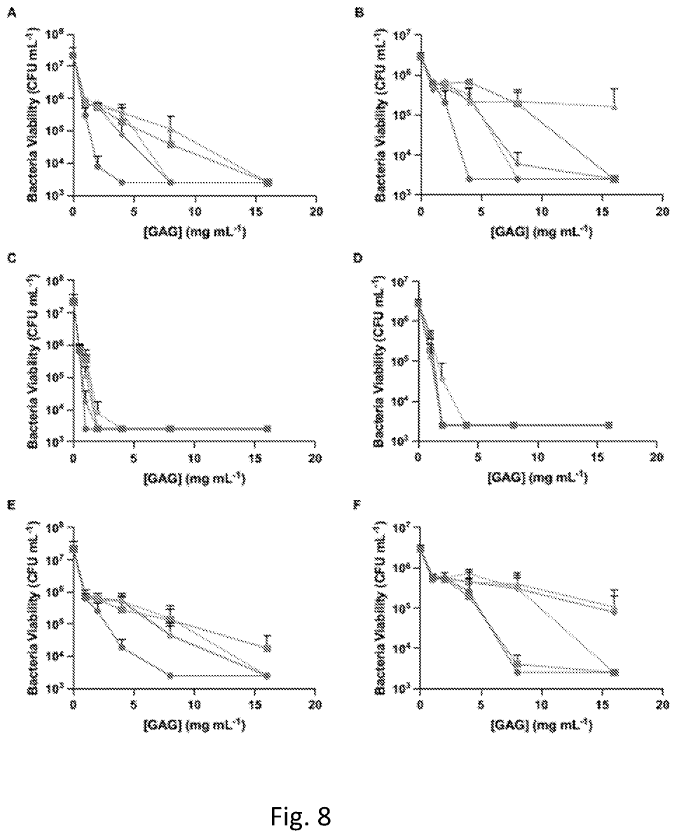

Bacteria were treated with a range of NO-releasing GAG concentrations under static conditions to determine the minimum bactericidal concentration, or MBC₄ₕ, defined as the concentration of GAG required to reduce bacterial viability by 3-log (i.e., >99.9%) over 4 h. While inhibition is beneficial over long-term exposures (i.e., 24 h), more rapid eradication is advantageous, as prior to eradication, bacteria will continue to replicate. As such, a 4-h exposure window was chosen to evaluate bactericidal properties. For all six bacteria strains, the NO-releasing DPTA-modified GAG derivatives were most effective at eradicating bacteria, requiring only 1-4 mg mL⁻¹ of the NO-releasing GAG for a 3-log reduction (FIGS. 6-8, Table 6). Greater concentrations of the HEDA- and DETA-modified NO-releasing GAG derivatives were required for P. aeruginosa and S. aureus, with the exception of HA6-HEDA/NO (Table 6). In comparing the molecular weights of HA, the 6 kDa HA was more effective than the 50 or 90 kDa derivatives, especially for AR-0239 and AR-0565 (FIG. 8), indicating that the lower molecular weight provides a beneficial property in faster diffusion to the bacteria and/or increased bacterial localization for NO delivery. The benefits of 6 kDa NO-releasing HA were reported on for eradicating biofilm-based P. aeruginosa over 90 kDa NO-releasing HA as a result of more rapid diffusion through the EPS matrix.²² This beneficial property may also translate to planktonic bacteria cultures. Significant differences in bactericidal activity were not observed between the two CS derivatives, which varied in sulfation pattern.

TABLE 6
Minimum bactericidal concentrations (MBC_{4 h}) of NO-releasing
glycosaminoglycan derivatives against antibiotic susceptible
and drug-resistant strains of common wound pathogens in
PBS following 4-h exposure.^{a}

| | MBC_{4 h} (mg mL⁻¹) | | | | | |
|---|---|---|---|---|---|---|
| | | | | ATCC | | |
| | | ATCC | AR- | S. | ATCC | AR- |
| Modification | PAO1 | MDR-PA | 0239 | aureus | MRSA | 0565 |
| HA6-HEDA/NO | 2 | 4 | 2 | 4 | 4 | 4 |
| HA50-HEDA/NO | 4 | 8 | 16 | 8 | 16 | >16 |
| HA90-HEDA/NO | 16 | 8 | 16 | 8 | 8 | 16 |
| CSA-HEDA/NO | 4 | 16 | 8 | 16 | 16 | 8 |
| CSC-HEDA/NO | 4 | >16 | 8 | 8 | 8 | 16 |
| HA6-DPTA/NO | 1 | 2 | 1 | 2 | 2 | 2 |
| HA50-DPTA/NO | 1 | 4 | 2 | 4 | 2 | 2 |
| HA90-DPTA/NO | 4 | 2 | 2 | 4 | 2 | 2 |
| CSA-DPTA/NO | 1 | 4 | 2 | 4 | 4 | 4 |
| CSC-DPTA/NO | 2 | 1 | 2 | 2 | 2 | 2 |
| HA6-DETA/NO | 4 | 4 | 4 | >16 | 8 | 8 |
| HA50-DETA/NO | 16 | 8 | 16 | 16 | 16 | >16 |
| HA90-DETA/NO | 4 | 8 | 16 | >16 | 8 | 16 |
| CSA-DETA/NO | 16 | >16 | 16 | >16 | 16 | 16 |
| CSC-DETA/NO | 4 | 16 | 16 | >16 | >16 | >16 |

^{a}MBC_{4 h} values were determined from n ≥ 3 separate experiments.

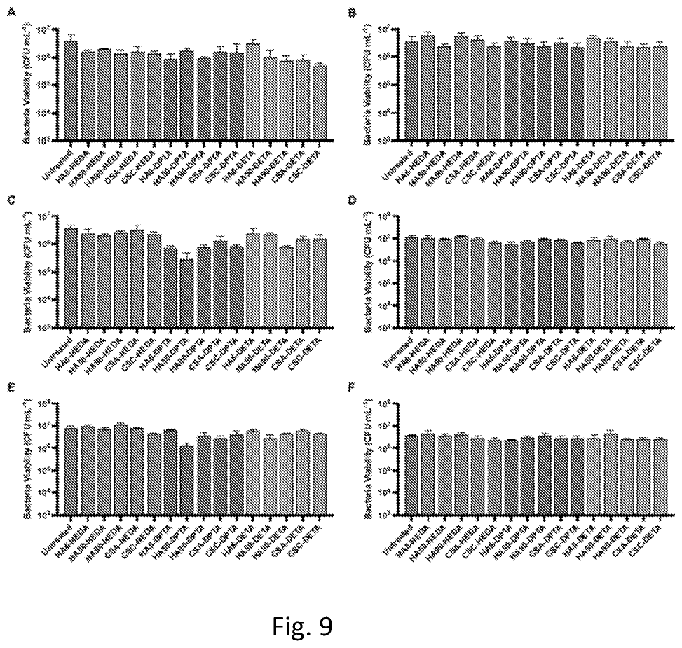

To confirm that NO was responsible for bactericidal activity rather than the biopolymer backbone, amine-modified GAGs (without NO) were evaluated against the six bacteria strains at 16 mg mL⁻¹ (FIG. 9). Negligible differences were observed for the three S. aureus strains (ATCC S. aureus, ATCC MRSA, and AR-0565), indicating that neither the GAG backbone nor amine modification were responsible for bactericidal activity (Figure insets B, D, and F in FIG. 9). For the P. aeruginosa derivatives, negligible decreases in bacteria viability were observed for HEDA-modified GAGs. However, a subset of the DPTA- and DETA-modified derivatives resulted in slight decreases in bacteria viability. For example, treatment with CSC-DETA resulted in a 0.89-log reduction in PAO1 viability (inset A in FIG. 9). Treatment with HA50-DPTA resulted in 1.12- and 0.79-log reductions in ATCC MDR-PA and AR-0239 viabilities, respectively (insets C and E in FIG. 9). However, this activity was significantly lower than the 3-log reduction threshold considered to be bactericidal, implicating NO as the bactericidal agent.

### Example 4: In Vitro Cytotoxicity Against Human Skin Cells

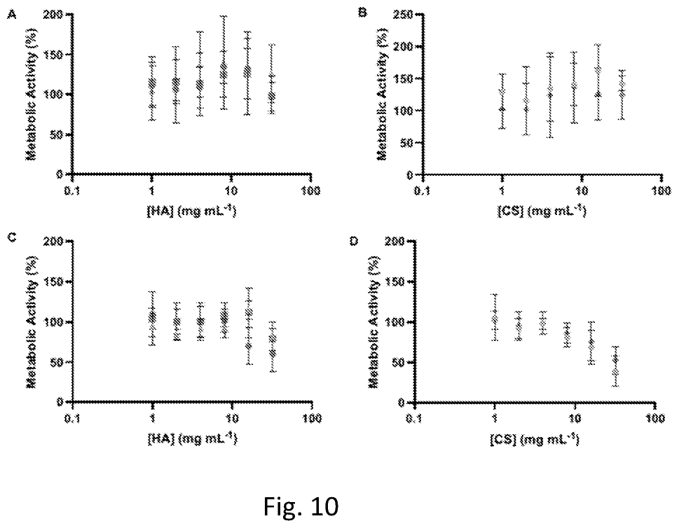

While antibacterial activity is a primary goal, minimizing off-target toxicity to mammalian cells is also important. In vitro cytotoxicity assays were performed using human dermal fibroblasts (HDFs) and human epidermal keratinocytes (HEKs) as representative cell types in the wound environment. Glycosaminoglycans are endogenously found and thus typically exhibit little-to-no toxicity; however, the effects of amine-modification and NO-loading on resulting toxicity to these cell types must still be considered. Fibroblasts and keratinocytes were dosed with a range of concentrations of unmodified GAGs, amine-modified GAGs, or NO-releasing GAGs to determine the IC₅₀ for each material, which is defined as the concentration of GAG that reduces the metabolic activity of the cells by 50% (FIGS. 10-12). In addition to allowing for toxicity comparisons between the derivatives, the IC₅₀ values help inform the upper limit for GAG concentrations utilized in further in vitro cell assays.

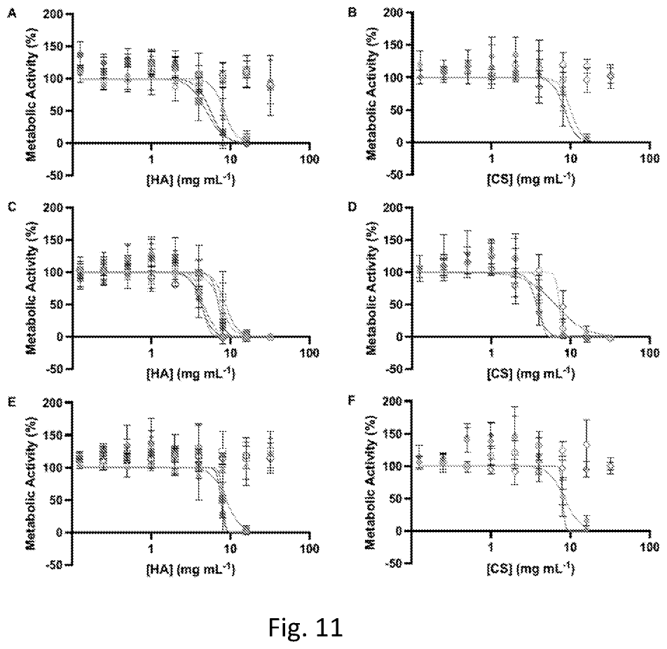

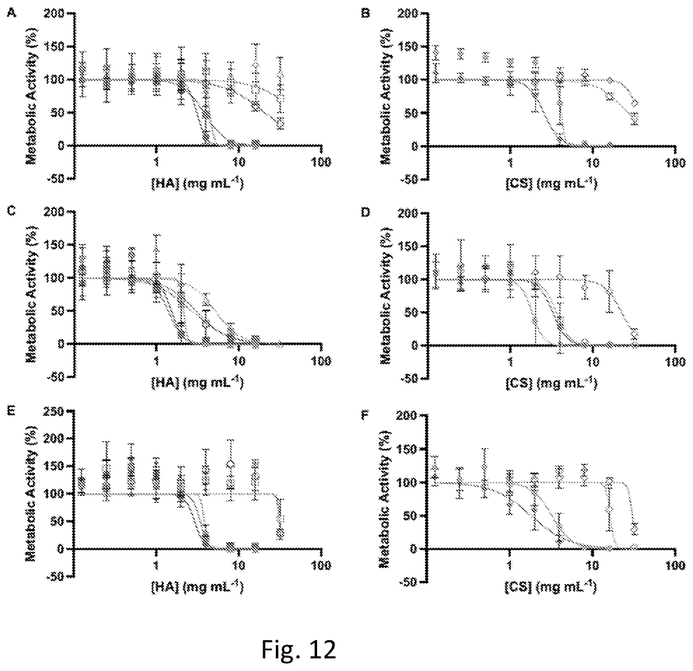

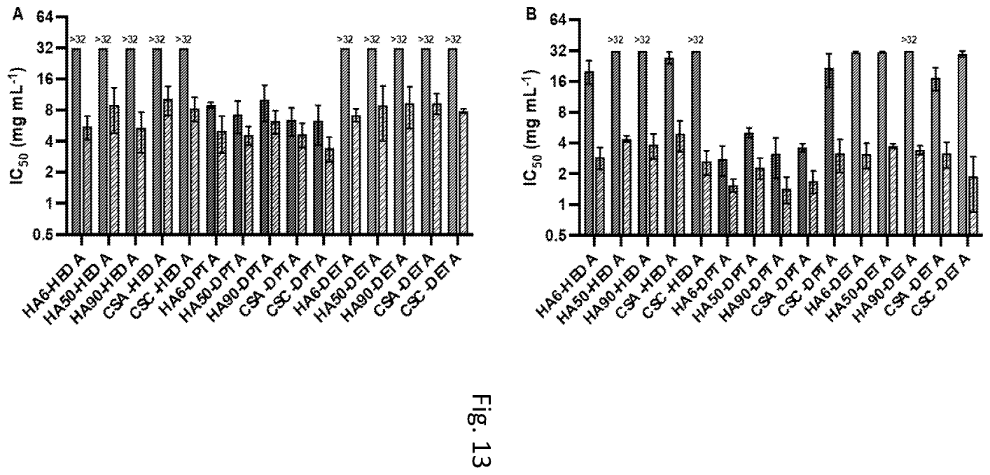

Unmodified GAG derivatives did not impact HDF cell viability at concentrations up to 32 mg mL⁻¹ (insets A and B in FIG. 10). However, a decrease in viability was found for HEKs upon treatment with 16-32 mg mL⁻¹ of unmodified HA scaffolds (60-80% activity) or unmodified CS scaffolds (40-80% activity), indicating that the HEKs are more susceptible to these materials than HDFs (insets C and D in FIG. 10). The amine-modified and NO-releasing GAG derivatives would thus be expected to have greater toxicity to HEKs than HDFs. Indeed, this toxicity is reflected in the resulting IC₅₀ values from the dose-response curves (FIGS. 9, 10, and 13). When evaluating HDFs (FIG. 11), both control HEDA- and DETA-modified GAGs (without NO) maintain ≥90% cell metabolic activity at concentrations up to 32 mg mL⁻¹, indicating that alkylamine modifications do not markedly change the toxicity profile at evaluated concentrations. A decrease in metabolic activity was observed for the same derivatives evaluated against HEKs at 16-32 mg mL⁻¹ (FIG. 12). For both cell types, DPTA modification led to greater cell toxicity, with IC₅₀ values of 6-10 mg mL⁻¹ and 2-5 mg mL⁻¹ (excluding CSC-DPTA) for HDFs and HEKs, respectively (FIG. 13). CSC-DPTA was the only DPTA-modified GAG derivative to exhibit an IC₅₀ against HEK in line with the other alkylamine modifications (IC₅₀ of 22±8 mg mL⁻¹).

Upon N-diazeniumdiolate NO donor formation, the GAG derivatives exhibited similar IC₅₀ values regardless of alkylamine, suggesting that the release of NO is strongly influential to the toxicity profile. For HDFs, NO-releasing DPTA-modified GAG biopolymers exhibited IC₅₀ values of 3-6 mg mL⁻¹, whereas the NO-releasing HEDA- and DETA-modified systems exhibited IC₅₀ values of 5-10 mg mL⁻¹. These overlapping ranges indicate that the NO loading on the material encompasses much of the toxicity profile, with lesser influence from the GAG backbone structure. For HEKs, the NO-releasing DPTA-modified GAG derivatives resulted in IC₅₀ values of 1-3 mg mL⁻¹ while the IC₅₀ values for NO-releasing HEDA- and DETA-modified derivatives were 1-5 mg mL⁻¹. As such, all further in vitro wound healing assays were performed using <1 mg mL⁻¹ GAG to ensure sufficient cell activity and survival. Although the cytotoxicity increased with NO donor formation, an in vitro monolayer of cells behaves differently than three-dimensional in vitro cell models or in vivo tissue. Indeed, monolayers of cells are often more susceptible to toxicity.^{75,76} Therefore, in vivo studies were utilized to confirm the therapeutic utility of the NO-releasing GAG derivatives at greater concentrations.

### Example 5: In Vitro TLR4 Inflammation Assay

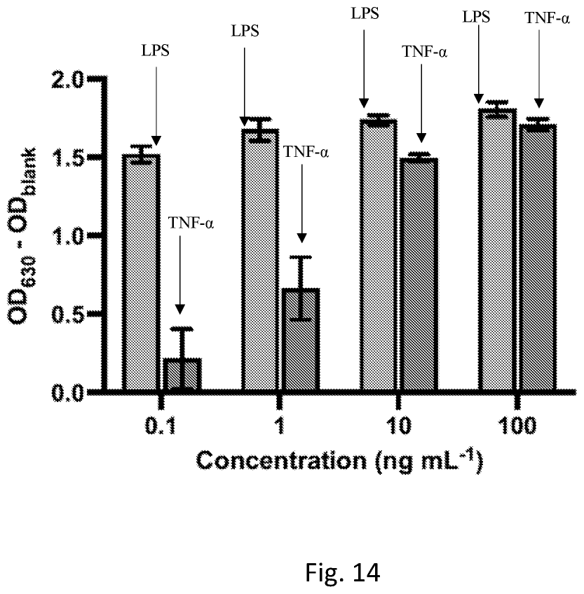

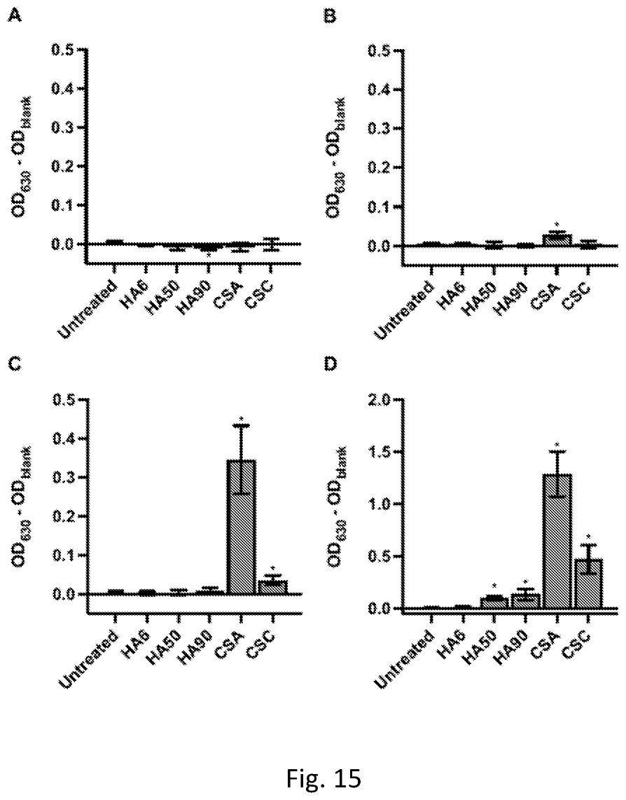

Inflammation is a central component to wound healing. Toll-like receptors (TLRs) are expressed on many cells that facilitate wound healing processes, including macrophages, neutrophils, fibroblasts, and keratinocytes. Toll-like receptor 4 (TLR4) is expressed by these four cell types, among others, and is shown to be associated with wound healing.⁷⁷ Hyaluronic acid fragments with a molecular weight of 10-250 kDa have been shown to interact with TLR4 during the inflammatory phase of wound healing.^{50,51} Stabler et al. reported that CS decreases NF-κB activation via TLR4 inhibition, although how sulfation pattern affects this process remains unclear.⁷⁸ The interactions between amine-modified and NO-releasing GAGs with TLR4 were thus evaluated using an in vitro assay that measures NF-κB-induced SEAP activity resulting from TLR4 activation on HEK-BLUE mTLR4 cells. Positive controls included TNF-α and LPS. TNF-α is a cytokine that interacts with many receptors leading to NF-κB activation. LPS activates NF-κB via TLR4 (FIG. 14).^{79,80} Unmodified GAGs were first evaluated at 100 ng mL⁻¹ to 100 μg mL⁻¹ using this assay. Increasing SEAP activity was observed with increasing concentration of GAGs (FIG. 15). At 100 μg mL⁻¹, both HA50 and HA90 exhibited significantly more NF-κB-induced SEAP production than untreated cells (inset D in FIG. 15). However, HA6 did not increase SEAP concentration, suggesting a molecular weight threshold for TLR4 activation. Both CSA and CSC interacted with TLR4, leading to activation at a greater level than that achieved with the HA derivatives. Of note, CSA exhibited greater activation of TLR4 than CSC, demonstrating the role that sulfation pattern plays in CS's inflammatory properties.

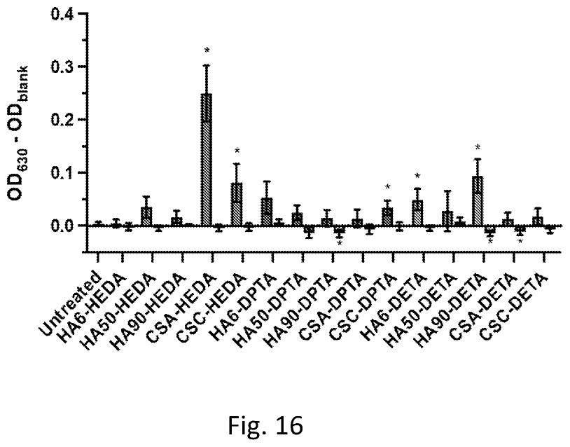

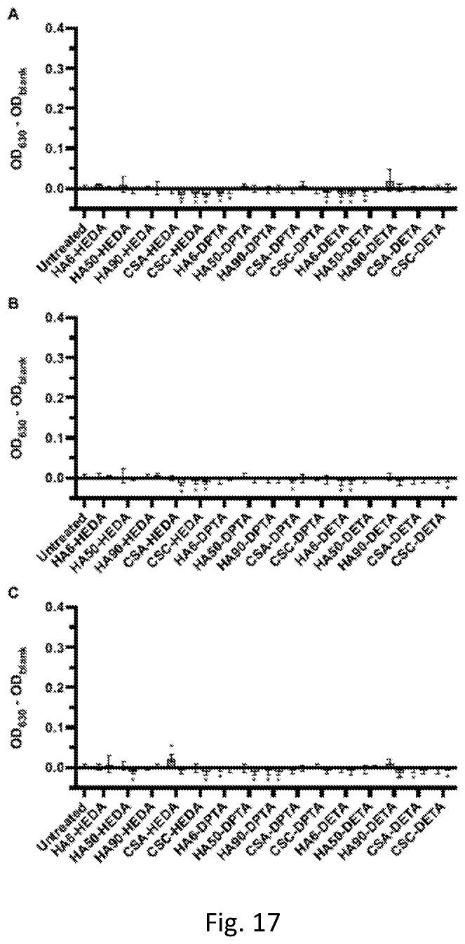

In evaluating amine-modified and NO-releasing GAGs, negligible increases in TLR4 activity were observed compared to untreated cells upon treatment of cells with 100 ng mL⁻¹ to 10 μg mL⁻¹, with the exception of CSA-HEDA at 10 μg mL⁻¹ (FIG. 17). At 100 μg mL⁻¹ GAG treatment doses (FIG. 16), five of the fifteen amine-modified GAGs significantly increased SEAP concentrations compared to untreated cells, indicating that these biopolymers maintained some interaction with TLR4, albeit at lesser levels than the unmodified GAGs. Regardless of the alkylamine used to form the NO donor, each NO-releasing GAG derivative exhibited negligible TLR4 activation at concentrations up to 100 μg mL⁻¹. By minimizing TLR4 activity, downstream activation of NF-κB should be lessened. Activation of NF-κB is an important component in the inflammatory process and associated with many inflammatory diseases. Decreased TLR4 activity as a result of NO release supports NO's roles as an anti-inflammatory agent.^{17,19}

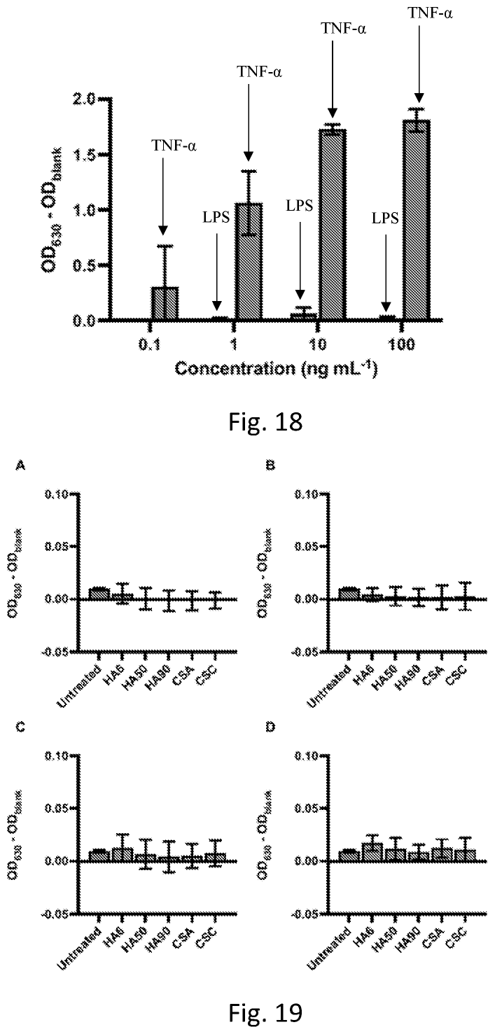

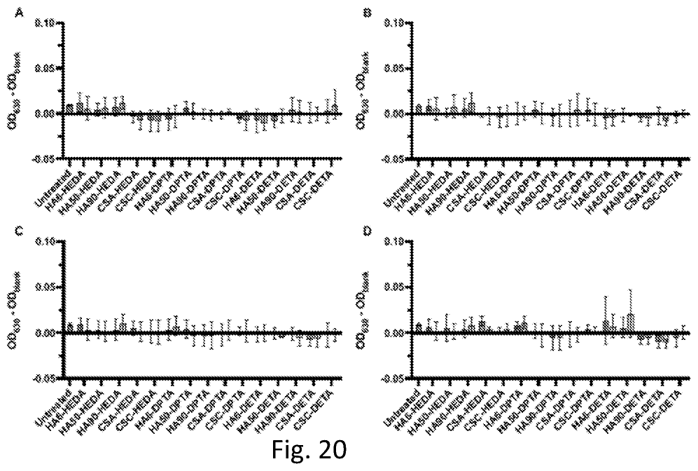

To ensure that the NF-κB activation resulted from TLR4 activity and not that of other receptors present at endogenous levels on the HEK-BLUE mTLR4 cells, HEK-BLUE Null1-v cells were evaluated in the same manner. These cells exhibit endogenous levels of TLR3, TLR5, NOD1, ALPK1, and TIFA receptors similar to HEK-BLUE mTLR4 cells but are not transfected with the murine TLR4 receptor gene. TNF-α was included as a positive control to confirm NF-κB activation, and LPS served as a negative control, as without TLR4 present, it does not cause NF-κB activation (FIG. 18). All GAG derivatives, whether unmodified, amine-modified, or NO-releasing, exhibited negligible NF-κB activation without TLR4 present (FIGS. 19 and 20), confirming that the activation or lack thereof of NF-κB by the GAG derivatives is the result of TLR4 activity. Unequivocally, NO release from NO donor-modified GAGs decreases TLR4 activity in vitro.

### Example 6: In Vitro Adhesion of Human Skin Cells to Extracellular Matrix Components

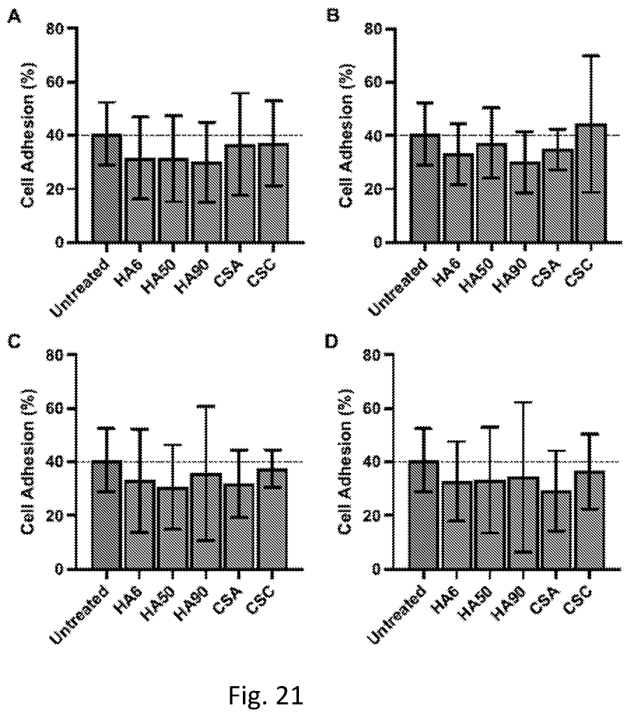

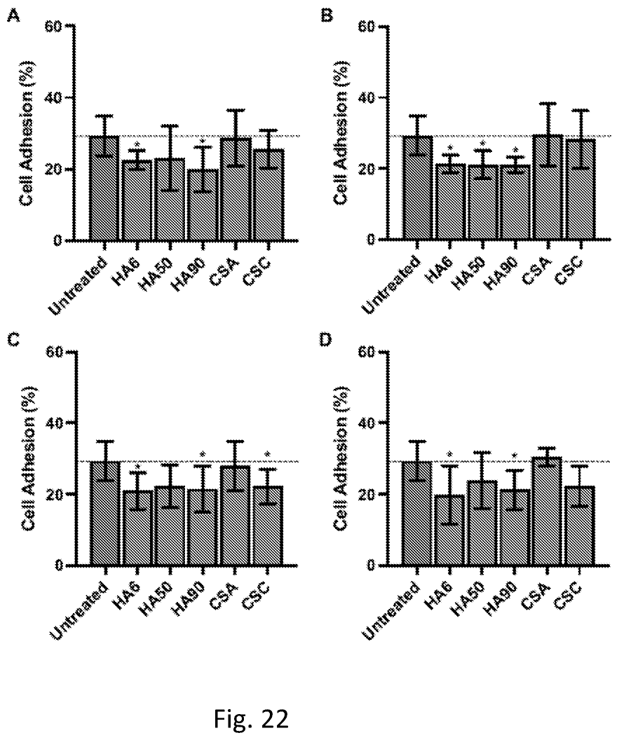

The ability of mammalian cells to adhere to ECM components and/or other cells is important for successful wound healing.⁷¹ The analysis of cell-ECM adhesion was evaluated using static adhesion assays by treating fibroblasts and keratinocytes with 100 ng mL⁻¹ to 100 μg mL⁻¹ of unmodified, amine-modified, or NO-releasing GAGs. Concurrent with treatment, the cells were seeded into collagen I-coated well plates, as collagen I is a major component of the ECM in the skin and facilitates adhesion. The effect of the GAGs on adhesion to collagen I was determined by monitoring the quantity of metabolically active cells adhered to the plate after multiple washes (removal of non-adhered cells). Adhesion of HDFs and HEKs was first evaluated in response to treatment with unmodified GAGs. Treatment of HDFs with unmodified HA or CS resulted in a slight decrease in adhesion to the collagen I-coated surface relative to untreated cells but was within error (FIG. 21). Treatment of the HEKs led to significantly less adhesion at one or more evaluated concentrations for all GAGs except for CSA (FIG. 22), indicating that the sulfation pattern of CS may impact adhesion of cells to ECM components.

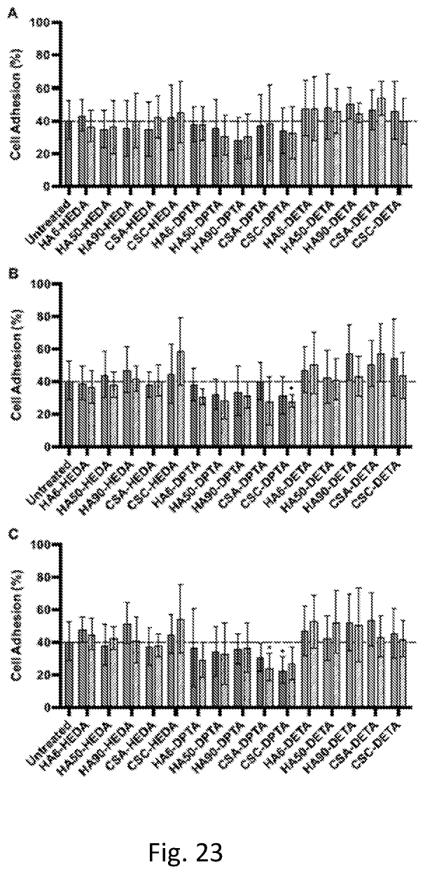

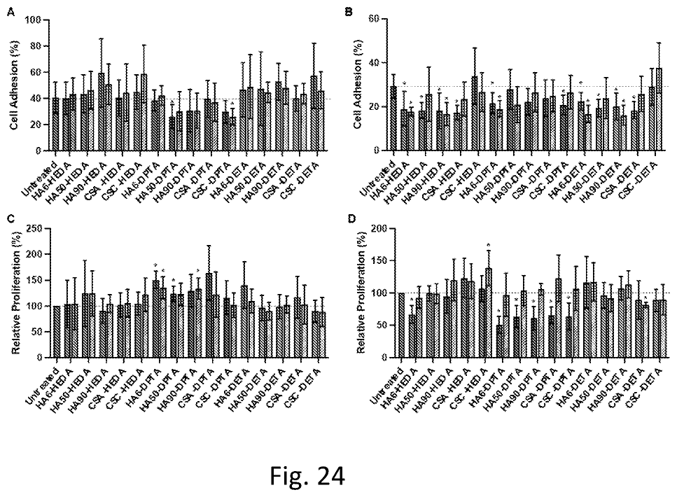

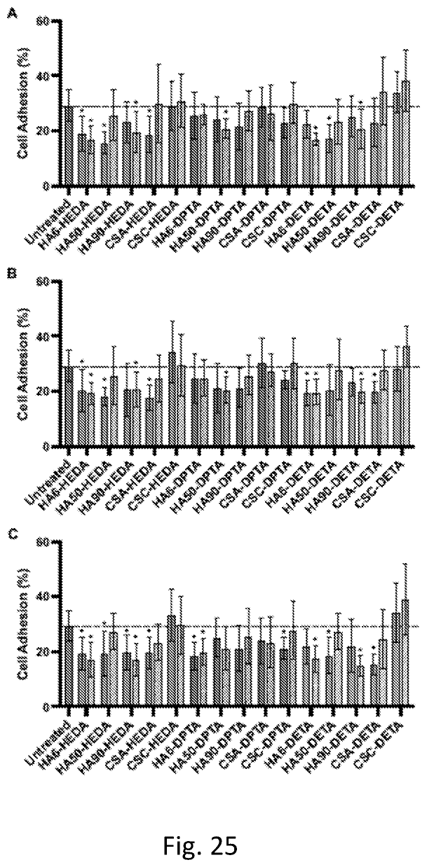

A decrease in HDF adhesion was only observed for DPTA-modified GAGs, both with and without NO, for treatments spanning 100 ng mL⁻¹ to 100 μg mL⁻¹ (FIG. 23, inset A in FIG. 24). HEDA- and DETA-modified GAGs were not significantly different than untreated cells. At two of the four evaluated concentrations, treatment with CSC-DPTA/NO significantly hindered cell adhesion. No major trends were observed regarding GAG identity, HA molecular weight, or CS sulfation pattern. The inhibition of adhesion was predominantly determined by alkylamine substituent. In contrast, treatment of HEKs with amine-modified and NO-releasing GAGs at the same concentrations demonstrated differences regarding GAG identity. The majority of the derivatives decreased HEK adhesion to ECM components at one or more of the evaluated concentrations (FIG. 25, inset B in FIG. 24). Treatment with each NO-releasing CSC derivative (CSC-HEDA/NO, CSC-DPTA/NO, and CSC-DETA/NO), however, did not influence cell adhesion to collagen I versus untreated cells. In terms of promoting or maintaining cell adhesion properties for both fibroblasts and keratinocytes, both CSC-HEDA and CSC-DETA were the only candidates exhibiting such behavior at all tested concentrations (with and without NO).

### Example 7: In Vitro Proliferation of Human Skin Cells

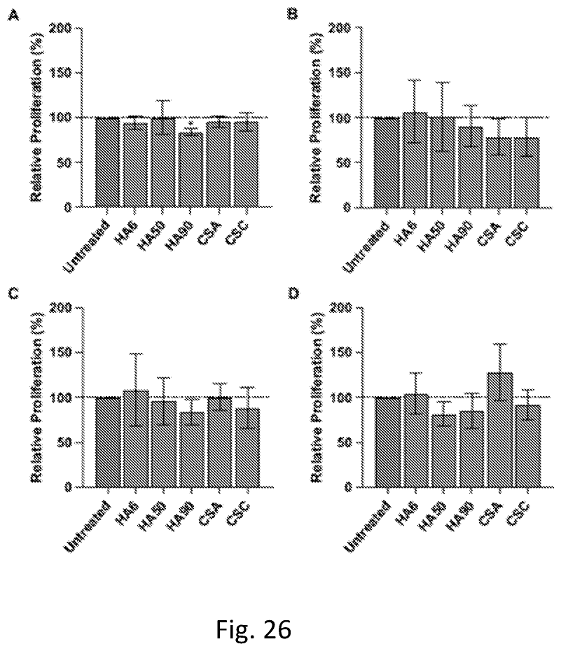

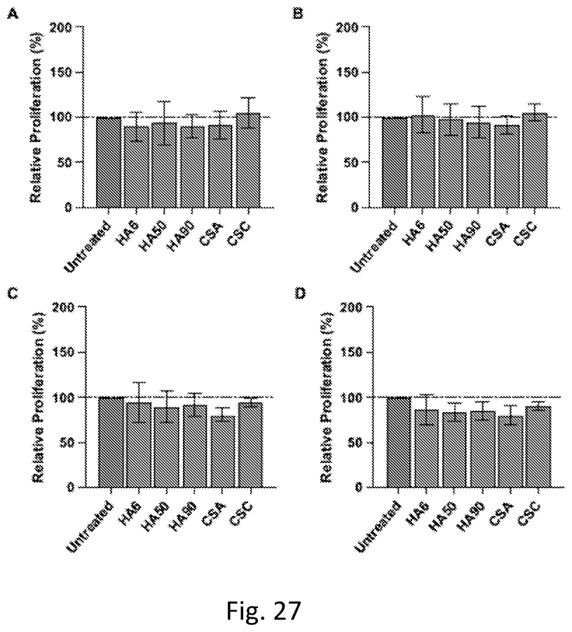

Following adhesion, cells such as fibroblasts and keratinocytes must proliferate in the wound environment. The proliferation of dermal fibroblasts is essential to wound healing, as these cells play key roles in the deposition of new ECM as well as wound contraction.⁸¹ Epidermal keratinocytes are involved in restoration of the epidermis; upon migration to the wound site, keratinocytes proliferate to begin filling the region of missing tissue.⁸² Due to the importance of cell proliferation, the effect of unmodified, amine-modified, and NO-releasing GAGs was evaluated using in vitro proliferation assays. The impact of unmodified materials on HDF and HEK proliferation was first investigated by treating both HDFs and HEKs with unmodified HA and CS. Treatment with unmodified HA90 and CSC resulted in slightly lower proliferation of HDFs at all concentrations tested versus untreated cells (FIG. 26). Each of the unmodified materials facilitated lower proliferation of HEKs at 10 or 100 μg mL⁻¹ (FIG. 27), indicating that the unmodified materials themselves do not facilitate significantly enhanced fibroblast and keratinocyte proliferation over 100 ng mL⁻¹ to 100 μg mL⁻¹.

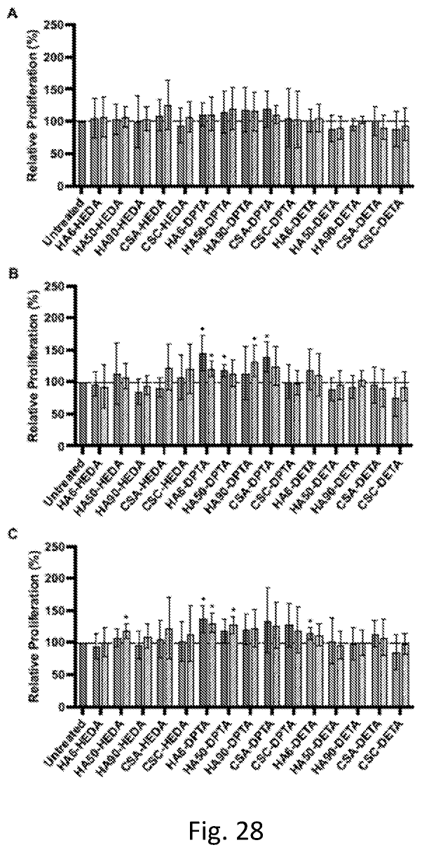

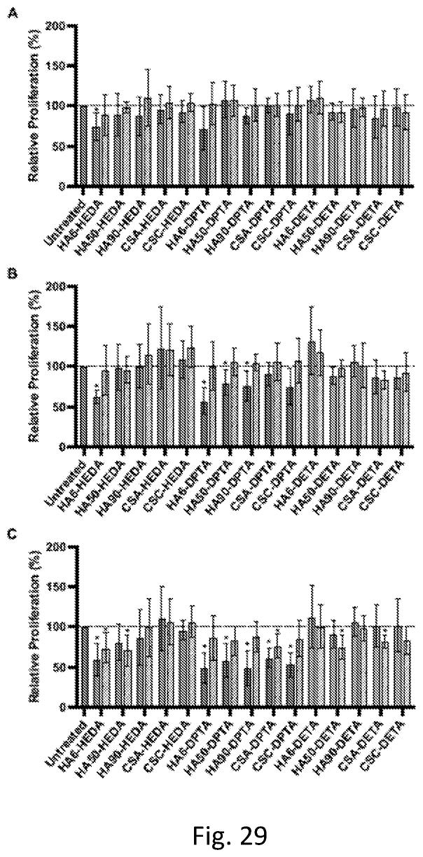

This effect was altered upon modification of the biopolymers with alkylamines and N-diazeniumdiolate NO donors. Treatment of the HDFs with HEDA- and DETA-modified GAGs with and without NO donors exhibited similar proliferation to untreated cells (FIG. 28, inset C in FIG. 24). However, the DPTA moiety, either alone or in addition to NO donor formation, facilitated similar or enhanced HDF proliferation compared to untreated cells. This enhancement is most clearly observed with HA6-DPTA and HA6-DPTA/NO, which significantly increase proliferation at 1-100 μg mL⁻¹ treatment doses. A different trend was observed when the same derivatives were used to treat HEKs. Nearly all of the HEDA- and DETA-modified GAGs, with and without NO, decreased or retained proliferation properties compared to untreated cells (FIG. 29, inset D in FIG. 24). The CSC-HEDA/NO enhanced cell proliferation at 10 μg mL⁻¹, making it the only GAG derivative to elevate proliferation at any of the evaluated concentrations (inset D in FIG. 24). Contrary to the proliferative effects observed for DPTA-modified GAGs against HDFs, the proliferation of HEKs lessened substantially over 1-100 μg mL⁻¹ for non-NO-releasing DPTA-modified GAGs. However, the modification of the DPTA-modified GAGs with NO was compensatory and allowed for proliferation to be restored to untreated levels when treated with 1-10 μg mL⁻¹. The contrasting effects of DPTA-modified GAGs motivated their further study in order to understand their impact on wound healing in a more complex model (i.e., containing both fibroblasts and keratinocytes). With respect to the materials that best promoted or maintained proliferation of HDFs and HEKs, only CSA-HEDA, CSC-HEDA, HA6-DETA, and HA90-DETA fulfilled this requirement for both amine-modified and NO-releasing forms.

### Example 8: In Vivo Evaluation of NO-Releasing Glycosaminoglycans on Infected Murine Wounds

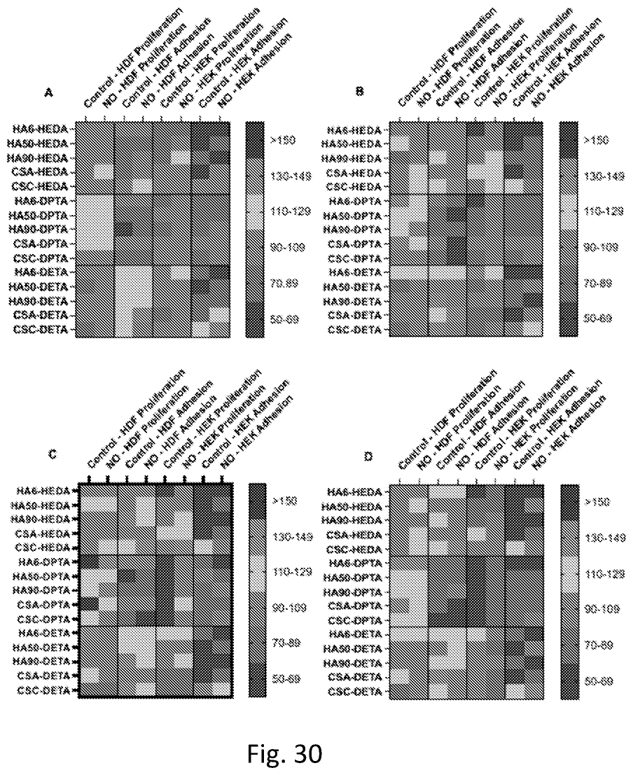

The in vitro assays described heretofore were utilized to narrow the list of wound healing therapeutic candidates for in vivo evaluation. The antibacterial experiments point to HA6-HEDA/NO along with all NO-releasing DPTA-modified GAGs as the most effective for eradicating multiple strains/resistance profiles of P. aeruginosa and S. aureus. When summarizing the proliferation and adhesion assays for HDFs and HEKs, CSC-HEDA and CSC-HEDA/NO were revealed as the only derivatives with all neutral or positive influences on proliferation and adhesion regardless of concentration (FIG. 30). HA6-HEDA/NO and CSC-HEDA/NO were thus selected as suitable therapeutic candidates for the initial in vivo wound healing study.

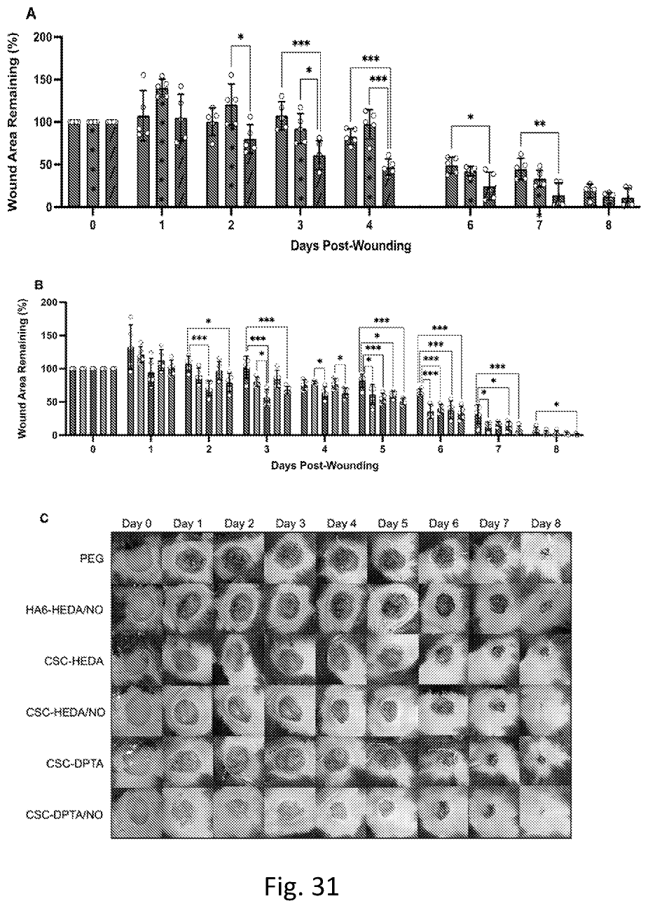

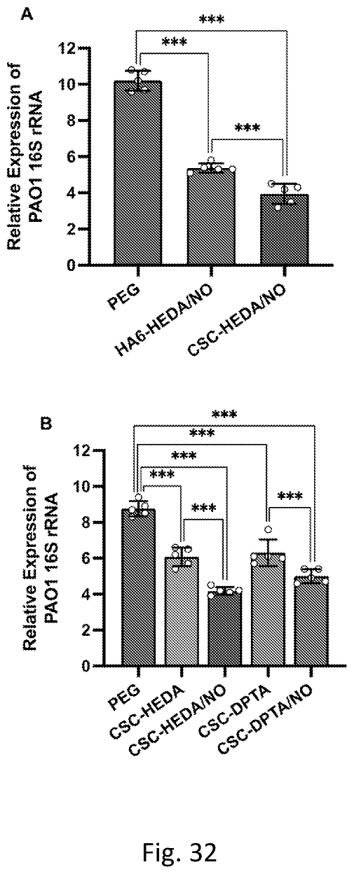

Mice were wounded using a standard biopsy punch procedure, with the wound inoculated with 2×10⁵ CFU of PAO1. Initial in vivo experiments included three treatment groups, with one group receiving vehicle only (i.e., PEG), a second group receiving HA6-HEDA/NO in PEG (50 mg kg⁻¹ body weight), and a third group receiving CSC-HEDA/NO in PEG (50 mg kg⁻¹ body weight). Mice were treated once daily beginning immediately following the wounding and infection procedure. Wounds were imaged daily and measured in perpendicular directions with calipers. The percentage of wound area remaining at each time point was calculated relative to the initial wound area to monitor wound closure. Wounds treated with HA6-HEDA/NO increased in size over the initial 24 h following wounding but then began to decrease in size (inset A in FIG. 31). Treatment with HA6-HEDA/NO did not significantly decrease remaining wound area compared to the PEG controls at any time point. In contrast, treatment of mice with CSC-HEDA/NO decreased remaining wound area compared to the PEG control on days 3 through 7 post-wounding. For example, CSC-HEDA/NO treated wounds retained ˜60% and ˜15% of their wound areas at day 3 and 7 post-wounding, respectively, whereas PEG-treated wounds exhibited ˜105% and ˜45% at the same timepoints. The CSC-HEDA/NO treatment also proved better (i.e., greater wound closure) than HA6-HEDA/NO on days 2 through 4 post-wounding. In comparing the remaining P. aeruginosa genome in the wound tissue at day 8 post-wounding, it was clear that the NO-releasing GAGs significantly decreased the bacterial burden (inset A in FIG. 32). Aligning with the wound closure results, treatment with CSC-HEDA/NO resulted in significantly less P. aeruginosa genome remaining at day 8 post-wounding than was found with HA6-HEDA/NO. The discrepancy between the two treatments regarding both wound closure and antibacterial efficacy cannot be attributed to NO payloads, as HA6-HEDA/NO and CSC-HEDA/NO released similar amounts of NO, 0.40±0.04 and 0.38±0.11 μmol NO mg⁻¹, respectively, in SWF. Further, CSC-HEDA/NO only releases NO for 2.9±0.8 h in SWF compared to 7.6±1.2 h for HA6-HEDA/NO (FIG. 5), suggesting that a longer release duration on its own is not beneficial. It was then decided to evaluate the therapeutic activity of the CSC-HEDA backbone (without NO) to determine how the biopolymer alone may influence wound healing.

While CSC-HEDA/NO promoted accelerated wound healing compared to HA6-HEDA/NO or the control group in vivo, it did not exhibit the strongest antibacterial properties in vitro. In a second in vivo study, CSC-DPTA/NO was included to determine the effect of greater antibacterial properties on wound healing. The inclusion of non-NO-releasing CSC-HEDA and CSC-DPTA controls were necessary to determine the role of the biopolymer scaffold alone. The following five treatment groups were evaluated: (1) vehicle only (i.e., PEG); (2) CSC-HEDA in PEG (50 mg kg⁻¹ body weight); (3) CSC-HEDA/NO in PEG (50 mg kg⁻¹ body weight); (4) CSC-DPTA in PEG (50 mg kg⁻¹ body weight); and, (5) CSC-DPTA/NO in PEG (50 mg kg⁻¹ body weight). As described above, the wounds were treated immediately following wounding/infection and once daily thereafter.

Beginning on day 2 post-wounding, both NO-releasing derivatives (i.e., CSC-HEDA/NO and CSC-DPTA/NO) were providing a substantial benefit to wound healing compared to PEG-treated (control) wounds (inset B in FIG. 31). On days 2 and 3 post-wounding, wound closure for the NO-releasing CSC biopolymers was significantly more advanced than the PEG-treated wounds. By day 4, the CSC-HEDA/NO and CSC-DPTA/NO demonstrated significantly greater wound closure than the CSC-HEDA and CSC-DPTA controls, highlighting the benefit to NO release, especially during the early wound healing phases, due to NO's ability to clear infection. This effect corroborates what is seen endogenously, where greater concentrations of NO are present at the initial stages of wound healing to assist in the inflammatory stage.²⁷ On days 5-7 post-wounding, treatments with NO-releasing and control GAGs were characterized by smaller wound areas relative to PEG controls, with no significant differences observed between the controls (i.e., amine-modified) and NO-releasing GAGs. On day 6 post-wounding, PEG-treated mice still exhibited 61-72% of their initial wounding area, whereas all mice treated with the GAGs had only 21-55% of their wound areas remaining. On day 8 post-wounding, residual wound tissue was analyzed via qPCR for residual PAO1 genetic material (inset B in FIG. 32). Mouse wounds treated with NO-releasing CS, modified with either HEDA or DPTA, contained significantly less PAO1 genome than those treated with CSC-HEDA, CSC-DPTA, or PEG, highlighting NO's influential antibacterial properties. However, wounds treated with non-NO-releasing CSC-HEDA and CSC-DPTA also exhibited significantly less PAO1 genome than control (i.e., PEG-treated) wounds, indicating that the CS backbone may promote endogenous antibacterial activities by supporting the wound healing pathway. While NO was most essential at the early stages, likely with regard to infection clearance, it is evident that the amine-modified GAG backbone also acts as a pro-wound healing agent itself. A portion of the early-stage success for the NO-releasing GAGs must therefore be attributed to the bioactive biopolymer backbone. At all points, no significant differences either quantitatively or visually were observed between wounds treated with CSC-HEDA/NO and CSC-DPTA/NO (inset C in FIG. 31), suggesting that the identity of the GAG (i.e., HA vs. CS) is more influential than the alkylamine substituent for murine wound healing. Of note, no adverse effects were observed in mice following treatment with any of the developed treatments. In all, the in vivo studies indicate that the NO-releasing CS derivatives exhibit pro-wound healing benefit contributions from both NO and the CS backbone.

### CONCLUSIONS

A series of glycosaminoglycans were modified with N-diazeniumdiolate NO donors to evaluate the role of native GAG properties (i.e., identity, molecular weight, sulfation pattern) and NO donor properties (i.e., alkylamine substituent, NO-release kinetics) on antibacterial activity and wound healing. The results of in vitro antibacterial assays demonstrated that DPTA-modified GAG derivatives exhibit superior bactericidal action that is not dependent on resistance mechanisms adopted by bacteria. As such, these materials represent a promising alternative to conventional antibiotics in treating antibiotic-resistant bacterial infections. The addition of NO release to GAG scaffolds decreased their activation profiles of TLR4, a receptor found on many wound-related cells that signals for pro-inflammatory activity, suggesting that these NO-releasing GAGs may exhibit further anti-inflammatory properties. Investigation of fibroblast and keratino-cyte adhesion and proliferation as a result of GAG treatment revealed CSC-HEDA/NO as an optimal in vitro pro-wound healing agent; however, major differences between HA and CS-based derivatives were not evident at this stage. An in vivo infected murine wound model analysis pointed to CS as a more beneficial backbone compared to HA, independent of HEDA or DPTA modification, for the development of an antibacterial wound healing agent. Further, the in vivo results support the idea that the antibacterial and wound healing benefits originate from both NO release and a bioactive CS backbone. The combination of these two entities has thus allowed for the development of a multi-functional antibacterial wound healing agent that demonstrates promise in addressing major complications related to chronic wounds.

### REFERENCES

(1) Sen, C. K.; Gordillo, G. M.; Roy, S.; Kirsner, R.; Lambert, L.; Hunt, T. K.; Gottrup, F.; Gurtner, G. C.; Longaker, M. T. Human Skin Wounds: A Major and Snowballing Threat to Public Health and the Economy. Wound Repair Regen. 2009, 17, 763-771.
(2) Sen, C. K. Human Wounds and Its Burden: An Updated Compendium of Estimates. Adv. Wound Care 2019, 8, 39-48.
(3) Järbrink, K.; Ni, G.; Sönnergren, H.; Schmidtchen, A.; Pang, C.; Bajpai, R.; Car, J. The Humanistic and Economic Burden of Chronic Wounds: A Protocol for a Systematic Review. Syst. Rev. 2017, 6, 1-7.
(4) Han, G.; Ceilley, R. Chronic Wound Healing: A Review of Current Management and Treatments. Adv. Ther. 2017, 34, 599-610.
(5) Stechmiller, J. K.; Childress, B.; Cowan, L. Arginine Supplementation and Wound Healing. 2005, 52-61.
(6) Guo, S.; DiPietro, L. A. Factors Affecting Wound Healing. J. Dent. Res. 2010, 89, 219-229.
(7) Malone-Povolny, M. J.; Maloney, S. E.; Schoenfisch, M. H. Nitric Oxide Therapy for Diabetic Wound Healing. Adv. Healthc. Mater. 2019, 8, 1801210.
(8) Gjødsbøl, K.; Christensen, J. J.; Karlsmark, T.; Jørgensen, B.; Klein, B. M.; Krogfelt, K. A. Multiple Bacterial Species Reside in Chronic Wounds: A Longitudinal Study. Int. Wound J. 2006, 3, 225-231.
(9) Bjarnsholt, T.; Kirketerp-Møller, K.; Jensen, P. Ø.; Madsen, K. G.; Phipps, R.; Krogfelt, K.; Høiby, N.; Givskov, M. Why Chronic Wounds Will Not Heal: A Novel Hypothesis. Wound Repair Regen. 2008, 16, 2-10.
(10) Percival, S. L.; Hill, K. E.; Williams, D. W.; Hooper, S. J.; Thomas, D. W.; Costerton, J. W. A Review of the Scientific Evidence for Biofilms in Wounds. Wound Repair Regen. 2012, 20, 647-657.
(11) Donlan, R. M.; Costerton, J. W. Biofilms: Survival Mechanisms of Clinically Relevant Microorganisms. Clin. Microbiol. Rev. 2002, 15, 167-193.
(12) Buch, P. J.; Chai, Y.; Goluch, E. D. Treating Polymicrobial Infections in Chronic Diabetic Wounds. Clin. Microbiol. Rev. 2019, 32, e00091-18.
(13) Ceri, H.; Olson, M. E.; Turner, R. J. Needed, New Paradigms in Antibiotic Development. Expert Opin. Pharmacother. 2010, 11, 1233-1237.
(14) Ventola, C. L. The Antibiotic Resistance Crisis: Part 1: Causes and Threats. Pharm. Ther. 2015, 40, 277-283.
(15) Tenover, F. C. Mechanisms of Antimicrobial Resistance in Bacteria. Am. J. Med. 2006, 119, S3-S10.
(16) Carpenter, A. W.; Schoenfisch, M. H. Nitric Oxide Release: Part II. Therapeutic Applications. Chem. Soc. Rev. 2012, 41, 3742-3752.
(17) Donnini, S.; Ziche, M. Constitutive and Inducible Nitric Oxide Synthase: Role in Angiogenesis. Antioxidants Redox Signal. 2002, 4, 817-823.
(18) Nathan, C. F.; Hibbs, J. B. Role of Nitric Oxide Synthesis in Macrophage Antimicrobial Activity. Curr. Opin. Immunol. 1991, 3, 65-70.
(19) Cirino, G.; Distrutti, E.; Wallace, J. L. Nitric Oxide and Inflammation. Inflamm. Allergy—Drug Targets 2006, 5, 115-119.
(20) Hetrick, E. M.; Shin, J. H.; Stasko, N. A.; Johnson, C. B.; Wespe, D. A.; Holmuhamedov, E.; Schoenfisch, M. H. Bactericidal Efficacy of Nitric Oxide-Releasing Silica Nanoparticles. ACS Nano 2008, 2, 235-246.
(21) Fang, F. C. Antimicrobial Reactive Oxygen and Nitrogen Species: Concepts and Controversies. Nat. Rev. Microbiol. 2004, 2, 820-832.
(22) Maloney, S. E.; McGrath, K. V.; Ahonen, M. J. R.; Soliman, D. S.; Feura, E. S.;
   Hall, H. R.; Wallet, S. M.; Maile, R.; Schoenfisch, M. H. Nitric Oxide-Releasing Hyaluronic Acid as an Antibacterial Agent for Wound Therapy. Biomacromolecules 2021, 22, 867-879.
(23) Ahonen, M. J. R.; Dorrier, J. M.; Schoenfisch, M. H. Antibiofilm Efficacy of Nitric Oxide-Releasing Alginates against Cystic Fibrosis Bacterial Pathogens. ACS Infect. Dis. 2019, 5, 1327-1335.
(24) Barraud, N.; Kelso, M.; Rice, S.; Kjelleberg, S. Nitric Oxide: A Key Mediator of Biofilm Dispersal with Applications in Infectious Diseases. Curr. Pharm. Des. 2014, 21, 31-42.
(25) Privett, B. J.; Broadnax, A. D.; Bauman, S. J.; Riccio, D. A.; Schoenfisch, M. H. Examination of Bacterial Resistance to Exogenous Nitric Oxide. Nitric Oxide 2012, 26, 169-173.
(26) Rouillard, K. R.; Novak, O. P.; Pistiolis, A. M.; Yang, L.; Ahonen, M. J. R.; McDonald, R. A.; Schoenfisch, M. H. Exogenous Nitric Oxide Improves Antibiotic Susceptibility in Resistant Bacteria. ACS Infect. Dis. 2021, 7, 23-33.
(27) Efron, D. T.; Most, D.; Barbul, A. Role of Nitric Oxide in Wound Healing. Curr. Opin. Clin. Nutr. Metab. Care 2000, 3, 197-204.
(28) Schairer, D. O.; Chouake, J. S.; Nosanchuk, J. D.; Friedman, A. J. The Potential of Nitric Oxide Releasing Therapies as Antimicrobial Agents. Virulence 2012, 3, 271-279.
(29) Neufeld, B. H.; Reynolds, M. M. Critical Nitric Oxide Concentration for Pseudomonas Aeruginosa Biofilm Reduction on Polyurethane Substrates. Biointerphases 2016, 11, 1-8.
(30) Cooke, J. P. NO and Angiogenesis. Atheroscler. Suppl. 2003, 4, 53-60.
(31) Morbidelli, L.; Donnini, S.; Ziche, M. Role of Nitric Oxide in the Modulation of Angiogenesis. Curr. Pharm. Des. 2005, 9, 521-530.
(32) Coleman, J. W. Nitric Oxide in Immunity and Inflammation. Int. Immunopharmacol. 2001, 1, 1397-1406.
(33) Witte, M. B.; Thornton, F. J.; Efron, D. T.; Barbul, A. Enhancement of Fibroblast Collagen Synthesis by Nitric Oxide. Nitric Oxide—Biol. Chem. 2000, 4, 572-582.
(34) Miller, C. C.; Miller, M. K.; Ghaffari, A.; Kunimoto, B. Treatment of Chronic Nonhealing Leg Ulceration with Gaseous Nitric Oxide: A Case Study. J. Cutan. Med. Surg. 2004, 8, 233-238.
(35) Ghaffari, A.; Jalili, R.; Ghaffari, M.; Miller, C.; Ghahary, A. Efficacy of Gaseous Nitric Oxide in the Treatment of Skin and Soft Tissue Infections. Wound Repair Regen. 2007, 15, 368-377.
(36) Yang, Y.; Qi, P. K.; Yang, Z. L.; Huang, N. Nitric Oxide Based Strategies for Applications of Biomedical Devices. Biosurface and Biotribology 2015, 1, 177-201.
(37) Weinberger, B.; Laskin, D. L.; Heck, D. E.; Laskin, J. D. The Toxicology of Inhaled Nitric Oxide. Toxilogical Sci. 2001, 59, 5-16.
(38) Hrabie, J. A.; Keefer, L. K. Chemistry of the Nitric Oxide-Releasing Diazeniumdiolate (“nitrosohydroxylamine”) Functional Group and Its Oxygen-Substituted Derivatives. Chem. Rev. 2002, 102, 1135-1154.
(39) Keefer, L. K. Fifty Years of Diazeniumdiolate Research: From Laboratory Curiosity to Broad-Spectrum Biomedical Advances. ACS Chem. Biol. 2011, 6, 1147-1155.
(40) Dashti, N.; Ansari, M.; Shabani, M.; Vardasti, S.; Mirsalehian, A. The Effect of Nitric Oxide Donor in Diabetic Wound Healing. Iran. J. Public Health 2003, 32, 59-63.
(41) Brisbois, E. J.; Bayliss, J.; Wu, J.; Major, T. C.; Xi, C.; Wang, S. C.; Bartlett, R. H.; Handa, H.; Meyerhoff, M. E. Optimized Polymeric Film-Based Nitric Oxide Delivery Inhibits Bacterial Growth in a Mouse Burn Wound Model. Acta Biomater. 2014, 10, 4136-4142.
(42) Riccio, D. A.; Schoenfisch, M. H. Nitric Oxide Release: Part I. Macromolecular Scaffolds. Chem. Soc. Rev. 2012, 41, 3731-3741.
(43) Lowe, A.; Bills, J.; Verma, R.; Lavery, L.; Davis, K.; Balkus, K. J. Electrospun Nitric Oxide Releasing Bandage with Enhanced Wound Healing. Acta Biomater. 2015, 13, 121-130.
(44) Masters, K. S. B.; Leibovich, S. J.; Belem, P.; West, J. L.; Poole-Warren, L. A. Effects of Nitric Oxide Releasing Poly(Vinyl Alcohol) Hydrogel Dressings on Dermal Wound Healing in Diabetic Mice. Wound Repair Regen. 2002, 10, 286-295.
(45) Melrose, J. Glycosaminoglycans in Wound Healing. Bone Tissue Regen. Insights 2016, 7, 29-50.
(46) Aya, K. L.; Stern, R. Hyaluronan in Wound Healing: Rediscovering a Major Player. Wound Repair Regen. 2014, 22, 579-593.
(47) Fallacara, A.; Baldini, E.; Manfredini, S.; Vertuani, S. Hyaluronic Acid in the Third Millennium. Polymers 2018, 10, 701.
(48) Zhao, L.; Liu, M.; Wang, J.; Zhai, G. Chondroitin Sulfate-Based Nanocarriers for Drug/Gene Delivery. Carbohydr. Polym. 2015, 133, 391-399.
(49) Schiraldi, C.; Cimini, D.; De Rosa, M. Production of Chondroitin Sulfate and Chondroitin. Appl. Microbiol. Biotechnol. 2010, 87, 1209-1220.
(50) Graça, M. F. P.; Miguel, S. P.; Cabral, C. S. D.; Correia, I. J. Hyaluronic Acid-Based Wound Dressings: A Review. Carbohydr. Polym. 2020, 241, 116364.
(51) Yang, H.; Song, L.; Zou, Y.; Sun, D.; Wang, L.; Yu, Z.; Guo, J. Role of Hyaluronic Acids and Potential as Regenerative Biomaterials in Wound Healing. ACS Appl. Bio Mater. 2021, 4, 311-324.
(52) Hussain, Z.; Thu, H. E.; Katas, H.; Nasir, S.; Bukhari, A.; Bukhari, S. N. A. Hyaluronic Acid-Based Biomaterials: A Versatile and Smart Approach to Tissue Regeneration and Treating Traumatic, Surgical, and Chronic Wounds. Polym. Rev. 2017, 57, 594-630.
(53) Gao, F.; Liu, Y.; He, Y.; Yang, C.; Wang, Y.; Shi, X.; Wei, G. Hyaluronan Oligosaccharides Promote Excisional Wound Healing through Enhanced Angiogenesis. Matrix Biol. 2010, 29, 107-116.
(54) Tolg, C.; Telmer, P.; Turley, E. Specific Sizes of Hyaluronan Oligosaccharides Stimulate Fibroblast Migration and Excisional Wound Repair. PLoS One 2014, 9, 1-10.
(55) Sattar, A.; Rooney, P.; Kumar, S.; Pye, D.; West, D. C.; Scott, I.; Ledger, P. Application of Angiogenic Oligosaccharides of Hyaluronan Increases Blood Vessel Numbers in Rat Skin. J. Invest. Dermatol. 1994, 103, 576-579.
(56) Vazquez, J. R.; Short, B.; Findlow, A. H.; Nixon, B. P.; Boulton, A. J. M.; Armstrong, D. G. Outcomes of Hyaluronan Therapy in Diabetic Foot Wounds. Diabetes Res. Clin. Pract. 2003, 59, 123-127.
(57) Hong, L.; Shen, M.; Fang, J.; Wang, Y.; Bao, Z.; Bu, S.; Zhu, Y. Hyaluronic Acid (HA)-Based Hydrogels for Full-Thickness Wound Repairing and Skin Regeneration. J. Mater. Sci. Mater. Med. 2018, 29, 1-11.
(58) Corsuto, L.; Rother, S.; Koehler, L.; Bedini, E.; Moeller, S.; Schnabelrauch, M.; Hintze, V.; Schiraldi, C.; Scharnweber, D. Sulfation Degree Not Origin of Chondroitin Sulfate Derivatives Modulates Keratinocyte Response. Carbohydr. Polym. 2018, 191, 53-64.
(59) Mikami, T.; Kitagawa, H. Biosynthesis and Function of Chondroitin Sulfate. Biochim. Biophys. Acta—Gen. Subj. 2013, 1830, 4719-4733.
(60) Lamari, F. N. The Potential of Chondroitin Sulfate as a Therapeutic Agent. Connect. Tissue Res. 2008, 49, 289-292.
(61) Kirker, K. R.; Luo, Y.; Nielson, J. H.; Shelby, J.; Prestwich, G. D. Glycosaminoglycan Hydrogel Films as Bio-Interactive Dressings for Wound Healing. Biomaterials 2002, 23, 3661-3671.
(62) Gilbert, M. E.; Kirker, K. R.; Gray, S. D.; Ward, P. D.; Szakacs, J. G.; Prestwich, G. D.; Orlandi, R. R. Chondroitin Sulfate Hydrogel and Wound Healing in Rabbit Maxillary Sinus Mucosa. Laryngoscope 2004, 114, 1406-1409.
(63) Pal, D.; Saha, S. Chondroitin: A Natural Biomarker with Immense Biomedical Applications. RSC Adv. 2019, 9, 28061-28077.
(64) Zou, X. H.; Foong, W. C.; Cao, T.; Bay, B. H.; Ouyang, H. W.; Yip, G. W. Chondroitin Sulfate in Palatal Wound Healing. J. Dent. Res. 2004, 83, 880-885.
(65) Zou, X. H.; Jiang, Y. Z.; Zhang, G. R.; Jin, H. M.; Hieu, N. T. M.; Ouyang, H. W. Specific Interactions between Human Fibroblasts and Particular Chondroitin Sulfate Molecules for Wound Healing. Acta Biomater. 2009, 5, 1588-1595.
(66) Kwon, H. J.; Han, Y. Chondroitin Sulfate-Based Biomaterials for Tissue Engineering. Turkish J. Biol. 2016, 40, 290-299.
(67) Scharnweber, D.; Hubner, L.; Rother, S.; Hempel, U.; Anderegg, U.; Samsonov, S. A.; Pisabarro, M. T.; Hofbauer, L.; Schnabelrauch, M.; Franz, S.; Simon, J.; Hintze, V. Glycosaminoglycan Derivatives: Promising Candidates for the Design of Functional Biomaterials. J. Mater. Sci. Mater. Med. 2015, 26, 232.
(68) Ahonen, M. J. R.; Suchyta, D. J.; Zhu, H.; Schoenfisch, M. H. Nitric Oxide-Releasing Alginates. Biomacromolecules 2018, 19, 1189-1197.
(69) Sutton Scott. Accuracy of Plate Counts. J. Valid. Technol. 2011, 17, 42-46.
(70) IUL. Eddy Jet 2 User's Guide. 2012, pp 1-50.
(71) Kucik, D. F.; Wu, C. Cell-Adhesion Assays. In Cell Migration: Developmental Methods and Protocols; Humana Press Inc.: Totowa, New Jersey, 2005; pp 43-54.
(72) Vigani, B.; Rossi, S.; Sandri, G.; Bonferoni, M. C.; Caramella, C. M.; Ferrari, F. Hyaluronic Acid and Chitosan-Based Nanosystems: A New Dressing Generation for Wound Care. Expert Opin. Drug Deliv. 2019, 16, 715-740.
(73) Jin, H.; Yang, L.; Ahonen, M. J. R.; Schoenfisch, M. H. Nitric Oxide-Releasing Cyclodextrins. J. Am. Chem. Soc. 2018, 140, 14178-14184.
(74) Jain, S.; Ohman, D. E. Role of an Alginate Lyase for Alginate Transport in Mucoid Pseudomonas Aeruginosa. Infect. Immun. 2005, 73, 6429-6436.
(75) Souza, A. G.; Silva, I. B. B.; Campos-Fernandez, E.; Barcelos, L. S.; Souza, J. B.;
   Marangoni, K.; Goulart, L. R.; Alonso-Goulart, V. Comparative Assay of 2D and 3D Cell Culture Models: Proliferation, Gene Expression and Anticancer Drug Response. Curr. Pharm. Des. 2018, 24, 1689-1694.
(76) Joseph, J. S.; Malindisa, S. T.; Ntwasa, M. Two-Dimensional (2D) and Three-Dimensional (3D) Cell Culturing in Drug Discovery. In Cell Culture; 2019; pp 21-42.
(77) Chen, L.; Dipietro, L. A. Toll-like Receptor Function in Acute Wounds. Adv. Wound Care 2017, 6, 344-355.
(78) Stabler, T. V.; Huang, Z.; Montell, E.; Vergés, J.; Kraus, V. B. Chondroitin Sulphate Inhibits NF-κB Activity Induced by Interaction of Pathogenic and Damage Associated Molecules. Osteoarthr. Cartil. 2017, 25, 166-174.
(79) Park, B. S.; Lee, J. O. Recognition of Lipopolysaccharide Pattern by TLR4 Complexes. Exp. Mol. Med. 2013, 45, e66.
(80) Moresco, E. M. Y.; LaVine, D.; Beutler, B. Toll-like Receptors. Curr. Biol. 2011, 21, R488-R⁴⁹³.
(81) Desjardins-Park, H. E.; Foster, D. S.; Longaker, M. T. Fibroblasts and Wound Healing: An Update. Regen. Med. 2018, 13, 491-495.
(82) Pastar, I.; Stojadinovic, O.; Yin, N. C.; Ramirez, H.; Nusbaum, A. G.; Sawaya, A.; Patel, S. B.; Khalid, L.; Isseroff, R. R.; Tomic-Canic, M. Epithelialization in Wound Healing: A Comprehensive Review. Adv. Wound Care 2014, 3, 445-464.

Unless defined otherwise, technical and scientific terms used herein have the same meaning as commonly understood by one of ordinary skill in the art to which this subject matter belongs.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A NO-releasing polymer compound comprising a unit structure of Formula I: 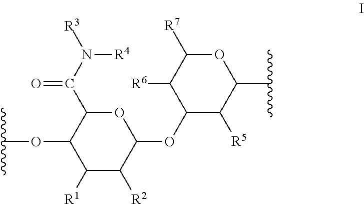
wherein,
R¹, R², and R⁵ are each independently selected from the group consisting of —OH, —NH₂, —CH₂OH, —C(O)OH, —NHC(O)—CH₃, C₁-C₆ alkoxy, —O—((CH₂)ₐO)_{b}—(CH₂)_{c}H, —NH—((CH₂)_{d}NH)ₑ—(CH₂)_{f}H, —X¹—((CH₂)_{g}X²)ₕ—(CH₂)ᵢH, —CH₂C(O)—X¹—((CH₂)_{g}X²)ₕ((CH₂)ⱼX³)ₖ(CH₂)ᵢH, —X¹—((CH₂)_{g}X²)ₕ((CH₂)ⱼX³)ₖ—(CH₂)ₗH, —X¹—(CH₂)_{g}X⁴, —CH₂C(O)—X¹—((CH₂)_{g}X²)ₕ(CH₂)ⱼX⁵, and —X¹—((CH₂)_{g}X²)ₕ(CH₂)ⱼX⁶;
R⁶ and R⁷ are each independently selected from the group consisting of —OH, —NH₂, —CH₂OH, —C(O)OH, C₁-C₆ alkoxy, —CH₂OSO₃⁻ and —OSO₃⁻ provided that at least one of R₆ and R₇ is —CH₂OSO₃⁻ or —OSO₃⁻;
R³ is selected from the group consisting of —((CH₂)_{g}X²)ₕ—(CH₂)ᵢH, —((CH₂)_{g}X²)ₕ((CH₂)ⱼX³)ₖ—(CH₂)ₗH, —(CH₂)_{g}X⁴, and —((CH₂)_{g}X²)ₕ(CH₂)ⱼX⁵;
R⁴ is selected from the group consisting of hydrogen and C₁-C₆ alkyl;
a, b, c, d, e, f, g, h, i, j, k, and l are each independently an integer between 0 and 10;
X¹, X², and X³ are each independently selected from the group consisting of —O—, —S—, —NH, —C(O)NH—, and a first NO donating moiety selected from the group consisting of 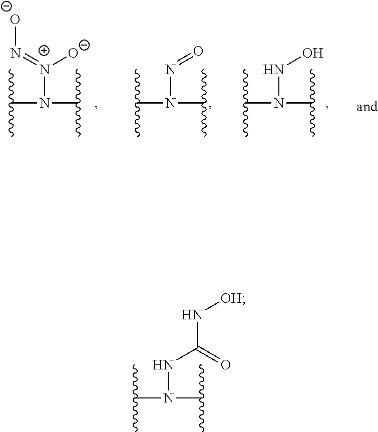
X⁴, X⁵, and X⁶ are each independently selected from a second NO donating moiety selected from the group consisting of 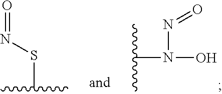
provided that the NO-releasing polymer compound contains at least one first NO donating moiety or second NO donating moiety.

2. The NO-releasing polymer compound of claim 1, wherein one of R⁶ and R⁷ is —CH₂OSO₃⁻ or —OSO₃⁻ and the other is —CH₂OH or —OH.

3. The NO-releasing polymer compound of claim 1, wherein R⁶ is —OSO₃⁻ and R⁷ is —CH₂OH.

4. The NO-releasing polymer compound of claim 1, wherein R⁷ is —CH₂OSO₃⁻ and R⁶ is —OH.

5. The NO-releasing polymer compound of claim 1, wherein R¹ and R² are each independently selected from the group consisting of —OH, —NH₂, —CH₂OH, —O—((CH₂)ₐO)_{b}—(CH₂)_{c}H, and —NH—((CH₂)_{d}NH)ₑ—(CH₂)_{f}H.

6. The NO-releasing polymer compound of claim 5, wherein R¹ and R² are each independently —OH.

7. The NO-releasing polymer compound of claim 1, wherein R⁵ is —NHC(O)—CH₃.

8. The NO-releasing polymer compound of claim 1, wherein R⁴ is hydrogen.

9. The NO-releasing polymer compound of claim 1, wherein the NO-releasing polymer compound contains the first NO-donating moiety

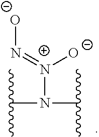 [SMILES: *N(*)/[N+]([O-])=N/[O-]]

10. The NO-releasing polymer compound of claim 1, wherein R³ is —((CH₂)_{g}X²)ₕ((CH₂)ⱼX³)ₖ—(CH₂)ₗH.

11. The NO-releasing polymer compound of claim 10, wherein:
g is 2;
X² is 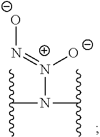 [SMILES: *N(*)/[N+]([O-])=N/[O-]]
h is 1;
j is 2;
X³ is O; and
l is 0.

12. The NO-releasing polymer compound of claim 10, wherein:
g is 3;
X² is 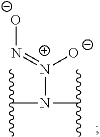 [SMILES: *N(*)/[N+]([O-])=N/[O-]]
h is 1;
j is 3;
X³ is NH; and
l is 0.

13. The NO-releasing polymer compound of claim 10, wherein:
g is 2;
X² is 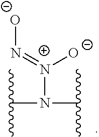 [SMILES: *N(*)/[N+]([O-])=N/[O-]]
h is 1;
j is 2;
X³ is NH; and
l is 0.

14. The NO-releasing polymer compound of claim 10, wherein R³ is selected from the group consisting of

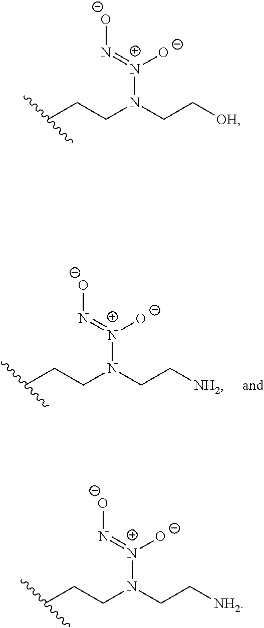

15. The NO-releasing polymer compound of claim 1, wherein the compound has a total releasable NO storage in a range of 0.1-1.0 μmol of NO per mg of compound (measured in 10 nM PBS at pH 7.4 at 37° C.).

16. The NO-releasing polymer compound of claim 1, wherein the compound has a NO half-life in the range of 0.1-24 hours (measured in 10 nM PBS at pH 7.4 at 37° C.).

17. The NO-releasing polymer compound of claim 1, wherein the compound has a total releasable NO storage in a range of 0.1-1.0 μmol of NO per mg of compound as measured in simulated wound fluid (10% v/v FBS in PBS at 37° C.).

18. The NO-releasing polymer compound of claim 17, wherein the compound has a total releasable NO storage in a range of 0.2-0.9 μmol of NO per mg of compound as measured in simulated wound fluid (10% v/v FBS in PBS at 37° C.).

19. The NO-releasing polymer compound of claim 1, wherein the compound has a NO half-life in the range of 0.1-24 hours as measured in simulated wound fluid (10% v/v FBS in PBS at 37° C.).

20. The NO-releasing polymer compound of claim 19, wherein the compound has a NO half-life in the range of 0.3-3 hours as measured in simulated wound fluid (10% v/v FBS in PBS at 37° C.).

21. A method of treating a wound infected with a bacterial pathogen, comprising contacting the wound with an effective amount of the NO-releasing polymer compound of claim 1.

22. The method of claim 21, wherein the bacterial pathogen is selected from the group consisting of P. aeruginosa and S. aureus.
